SEQUENCE LISTING (39 sequences)

SEQ ID NO: 1 - protein, 9 aa, synthetic construct
XWSHPQFEK

SEQ ID NO: 2 - protein, 9 aa, synthetic construct
XWHSPQFEK

SEQ ID NO: 3 - protein, 10 aa, synthetic construct
MIWSHPQFEK

SEQ ID NO: 4 - protein, 11 aa, synthetic construct
MWSHPQFEKST

SEQ ID NO: 5 - protein, 12 aa, synthetic construct
MRWSHPQFEKDR

SEQ ID NO: 6 - protein, 9 aa, synthetic construct
MWSHPQFEK

SEQ ID NO: 7 - protein, 12 aa, synthetic construct
XZWHSPQFEKST

SEQ ID NO: 8 - protein, 13 aa, synthetic construct
XZWHSPQFEKSXZ

SEQ ID NO: 9 - protein, 10 aa, synthetic construct
XZWHSPQFEK

SEQ ID NO: 10 - DNA, 1793 bp, synthetic construct
  source 1..1793: mol_type = other DNA
ggatcctgcagttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacgaattcagatctcgatcccgcgaaattaatacgactcactatagggagaccacaacggtttccctctagaaataattttgtttaactttaagaaggagatatacatatgatctggtctcatccgcagttcgaaaaatagtaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgt

SEQ ID NO: 11 - DNA, 36 bp, synthetic construct
  source 1..36: mol_type = other DNA
atgtggtctcatccgcagttcgaaaaatccacctag

SEQ ID NO: 12 - DNA, 45 bp, synthetic construct
  source 1..45: mol_type = other DNA
atgcgttggtctcatccgcagttcgaaaaatccaccgaccgttag

SEQ ID NO: 13 - DNA, 36 bp, synthetic construct
  source 1..36: mol_type = other DNA
gtaatacgactcactataggatcgaaagatttccgc

SEQ ID NO: 14 - DNA, 45 bp, synthetic construct
  source 1..45: mol_type = other DNA
acctaacgctaatcccctttcggggccgcggaaatctttcgatcc

SEQ ID NO: 15 - DNA, 22 bp, synthetic construct
  source 1..22: mol_type = other DNA
ggcgtaatacgactcactatag

SEQ ID NO: 16 - DNA, 18 bp, synthetic construct
  source 1..18: mol_type = other DNA
acctaacgctaatcccct

SEQ ID NO: 17 - DNA, 46 bp, synthetic construct
  source 1..46: mol_type = other DNA
acctaacgccatgtaccctttcggggatgcggaaatctttcgatcc

SEQ ID NO: 18 - DNA, 19 bp, synthetic construct
  source 1..19: mol_type = other DNA
acctaacgccatgtaccct

SEQ ID NO: 19 - DNA, 47 bp, synthetic construct
  source 1..47: mol_type = other DNA
acctaacgccacttacccctttcgcgggtgcggaaatctttcgatcc

SEQ ID NO: 20 - DNA, 19 bp, synthetic construct
  source 1..19: mol_type = other DNA
acctaacgccacttacccc

SEQ ID NO: 21 - DNA, 49 bp, synthetic construct
  source 1..49: mol_type = other DNA
gtaatacgactcactataggcggggtggagcagcctggtagctcgtcgg

SEQ ID NO: 22 - DNA, 38 bp, synthetic construct
  source 1..38: mol_type = other DNA
tggttgcgggggccggatttgaaccgacgatcttcggg

SEQ ID NO: 23 - DNA, 54 bp, synthetic construct
  source 1..54: mol_type = other DNA
gtaatacgactcactatagggtgattggcgcagcctggtagcgcacttcgttga

SEQ ID NO: 24 - DNA, 42 bp, synthetic construct
  source 1..42: mol_type = other DNA
cgaacccctgaccccttcgttatcaacgaagtgcgctaccag

SEQ ID NO: 25 - DNA, 38 bp, synthetic construct
  source 1..38: mol_type = other DNA
tggcgggtgataggggattcgaacccctgaccccttcg

SEQ ID NO: 26 - DNA, 55 bp, synthetic construct
  source 1..55: mol_type = other DNA
gtaatacgactcactatagggtgattggcgcagcctggtagcgcacttcgttacg

SEQ ID NO: 27 - DNA, 42 bp, synthetic construct
  source 1..42: mol_type = other DNA
cgaacccctgaccccttcgttcgtaacgaagtgcgctaccag

SEQ ID NO: 28 - DNA, 55 bp, synthetic construct
  source 1..55: mol_type = other DNA
gtaatacgactcactatagggtgattggcgcagcctggtagcgcacttcgttgtc

SEQ ID NO: 29 - DNA, 42 bp, synthetic construct
  source 1..42: mol_type = other DNA
cgaacccctgaccccttcgttgacaacgaagtgcgctaccag

SEQ ID NO: 30 - DNA, 49 bp, synthetic construct
  source 1..49: mol_type = other DNA
gtaatacgactcactataggctctgtagttcagtcggtagaacggcgga

SEQ ID NO: 31 - DNA, 64 bp, synthetic construct
  source 1..64: mol_type = other DNA
tggcggctctgactggactcgaaccagtgacatacggattgacagtccgccgttctaccgactg

SEQ ID NO: 32 - DNA, 19 bp, synthetic construct
  source 1..19: mol_type = other DNA
gtaatacgactcactatag

SEQ ID NO: 33 - DNA, 18 bp, synthetic construct
  source 1..18: mol_type = other DNA
tggcggctctgactggac

SEQ ID NO: 34 - DNA, 64 bp, synthetic construct
  source 1..64: mol_type = other DNA
gtaatacgactcactatagtccccttcgtctagaggcccaggacaccgccctacgaaggcggta

SEQ ID NO: 35 - DNA, 52 bp, synthetic construct
  source 1..52: mol_type = other DNA
tggcgtcccctaggggattcgaacccctgttaccgccttcgtagggcggtgt

SEQ ID NO: 36 - DNA, 20 bp, synthetic construct
  source 1..20: mol_type = other DNA
tggcgtcccctaggggattc

SEQ ID NO: 37 - DNA, 53 bp, synthetic construct
  source 1..53: mol_type = other DNA
ggcgtaatacgactcactatagtggaagaccccgtggagctcttcggagttac

SEQ ID NO: 38 - DNA, 41 bp, synthetic construct
  source 1..41: mol_type = other DNA
tcagcctgttatccccggggtaactccgaagagctccacgg

SEQ ID NO: 39 - DNA, 42 bp, synthetic construct
  source 1..42: mol_type = other DNA
accagaggtgccaaacctcacgatcagcctgttatccccggg

(19) United States
(12) Patent Application Publication
(10) Pub. No.: US 2025/0074944 A1
(43) Pub. Date: Mar. 6, 2025

(54) ACYLATED tRNA, INTRODUCTION OF PHARMACOLOGICALLY ACTIVE MOTIF IN PEPTIDE USING RIBOSOME CATALYSIS AND METHOD OF PREPARING SAME

(71) Applicant: POSTECH RESEARCH AND BUSINESS DEVELOPMENT FOUNDATION, Pohang-si (KR)

(72) Inventors: Joongoo LEE, Pohang-si (KR); Kanghun LEE, Pohang-si (KR)

(21) Appl. No.: 18/796,506

(22) Filed: Aug. 7, 2024

(30) Foreign Application Priority Data
Aug. 30, 2023 (KR) 10-2023-0114636
Jul. 19, 2024 (KR) 10-2024-0095918

(51) Int. Cl. C07K 7/64; C12N 15/11

(52) U.S. Cl. CPC C07K 7/64; C12N 15/11; C12N 2310/353

(57) ABSTRACT

Non-canonical substrates-acylated tRNA, introduction of non-canonical substrates into a peptide using ribosome catalysis, and method of preparing pharmacologically active motifs are disclosed. The present invention relates to acylated tRNA and introduction of pharmacologically active motif in peptide using ribosome-mediated catalysis and method that enables the production of a peptide having a cyclic motif without a complicated chemical process on an in vitro protein translation system.

### CROSS REFERENCE TO RELATED APPLICATION

The present application claims priority to Korean Patent Applications No. 10-2023-0114636, filed 30 August, 2023, and 10-2024-0095918, filed 19 July, 2024, the entire contents of which is incorporated herein for all purposes by this reference.

### REFERENCE TO AN ELECTRONIC SEQUENCE LISTING

The contents of the electronic sequence listing (DPH20240316US SEQ.xml; Size: 37,684 bytes; and Date of Creation: Oct. 15, 2024) is herein incorporated by reference in its entirety. The contents of the electronic sequence listing in no way introduces new matter into the specification.

### BACKGROUND OF THE DISCLOSURE

### 1. Field of the Disclosure

The present invention relates to a peptide having a pharmacologically active motif using acylated tRNA and a ribosome catalyst on a cell-free system, and a method for producing the same.

### 2. DESCRIPTION OF THE RELATED ART

Research using Flexizyme, a type of aminoacylating ribozyme, has utilized it as an accelerator to expand the candidate group of new drug substances by introducing various types of non-natural amino acids into the peptide. In particular, the strategy of producing macrocyclic peptides through chemical reactions of non-natural amino acids has attracted attention as a key element technology for new drug discovery. Macrocyclic peptides are cyclic peptides composed of about 10 amino acids and are attracting attention due to their high pharmacological effects and stability.

Meanwhile, three major strategies are adopted to introduce a cyclic motif into a peptide. First, there is the formation of a cyclic motif using an enzyme, second, there is the modification of a functional group through a chemical reaction after the introduction of a non-natural amino acid, and third, there is a combined reaction of an enzyme and a chemical reaction.

However, for all strategies, these designs are complex because they require substrates to be designed while considering various biochemical reactivity, and they do not guarantee a wide range of diversity expansion.

Therefore, research is needed on the synthesis of highly efficient ring-shaped motifs that ensure diversity expansion without complicating the process.

In addition, International Publication No. WO 2022/173627 A2 was disclosed as a prior art patent.

### SUMMARY OF THE DISCLOSURE

The purpose of the present invention is to provide a cell-free protein synthesis system in which non-natural amino acids having ester and hydrazino structures are sequentially introduced into a peptide, and a secondary reaction occurs between residues of the non-natural amino acids to form a square or octagonal structure, or various cyclic compounds, but not limited thereto.

Another purpose of the present invention is to provide a cell-free protein synthesis system in which a highly efficient cyclic motif can be quickly and easily obtained in vitro without going through a complex chemical process.

According to one aspect of the present invention, there is provided an acylated tRNA represented by Structural Formula 1 or Structural Formula 2 below:

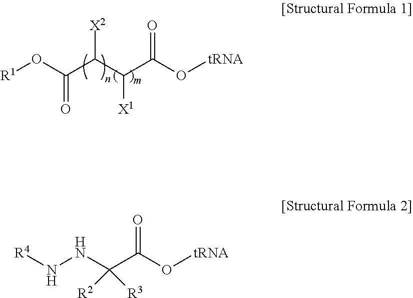

wherein R¹ is a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group, or a substituted or unsubstituted (C6-C14 aryl) (C1-C8 alkyl) group, X¹ and X² are each independently a hydrogen atom, a substituted or unsubstituted C1-C10 alkyl group, a substituted or unsubstituted C3-C7 cyclic alkyl group,

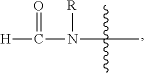

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the R¹, X¹ and X², and R⁴ is identical or different and represents a C1-C5 alkyl group or C6-C12 aryl group, and a group suitable for substitution on the R² and R³ is identical or different and represents a C1-C5 alkyl group, C6-C12 aryl group, a hydroxyl group or an amino acid group.

In addition, the acylated tRNA may be represented by the Structural Formula 1 and may comprise at least one selected from the group consisting of compounds below:

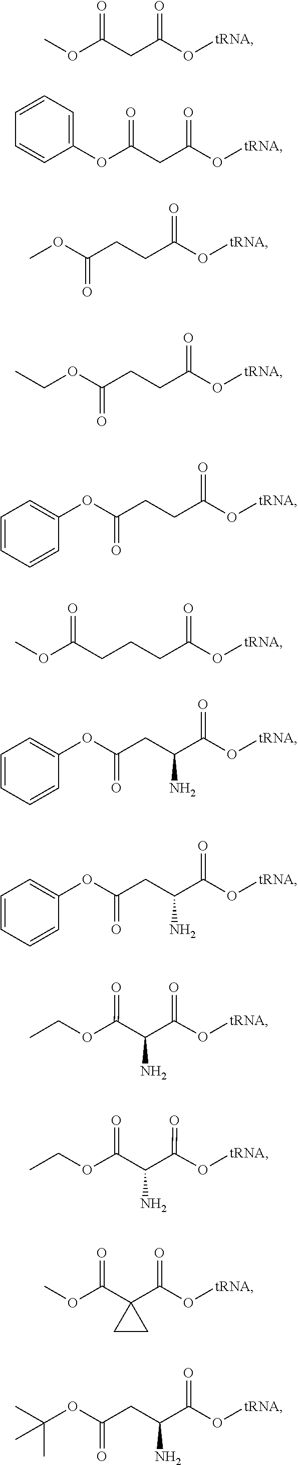

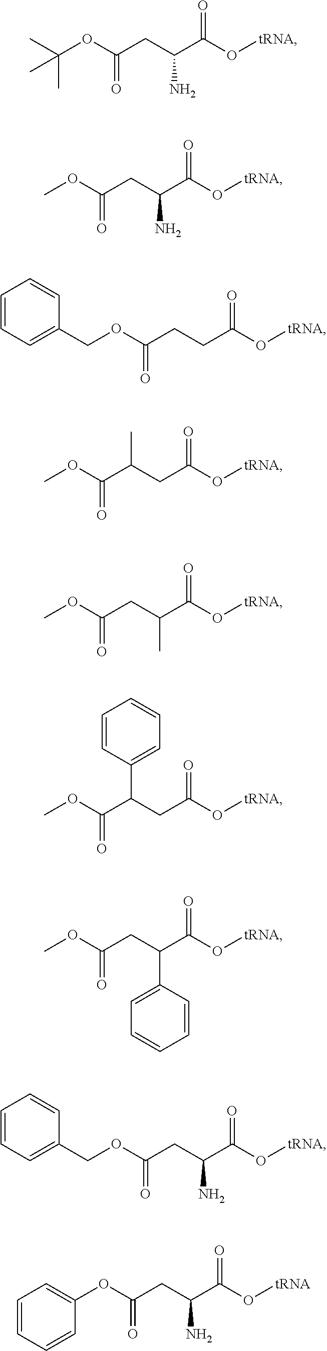

In addition, the acylated tRNA may be represented by the Structural Formula 2 and may comprise at least one selected from the group consisting of compounds below:

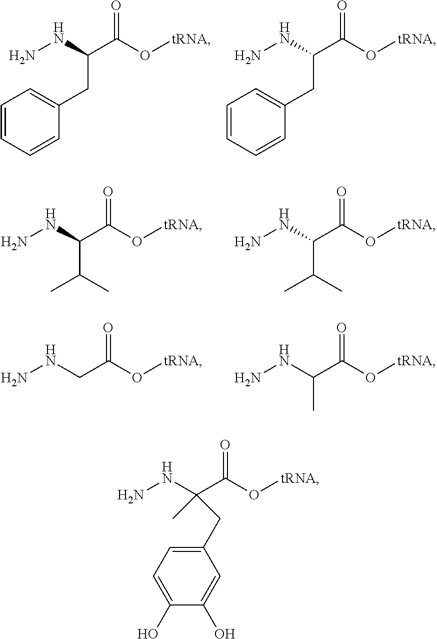

In addition, the acylated tRNA may represented by the Structural Formula 2 and comprises at least one selected from the group consisting of compounds below:

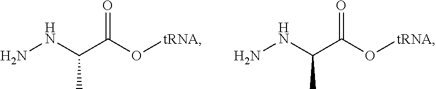

In addition, the acylated tRNA may be represented by the Structural Formula 2 and may comprises at least one selected from the group consisting of compounds below:

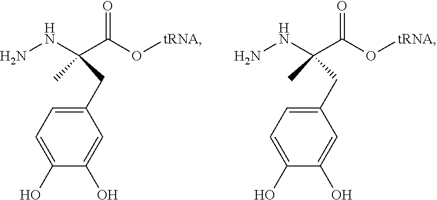

According to another aspect of the present invention, there is provided a cyclic compound represented by structural formula 3 below:

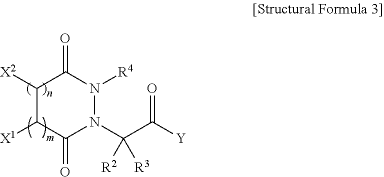

wherein X¹ and X² are each independently a hydrogen atom, a substituted or unsubstituted C1-C10 alkyl group, a substituted or unsubstituted C3-C7 cyclic alkyl group,

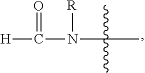

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², R⁴ and R⁵ to R⁷ is identical or different and represents a C1-C5 alkyl group or C6-C12 aryl group, and a group suitable for substitution on the R² and R³ is identical or different and represents a C1-C5 alkyl group, C6-C12 aryl group, a hydroxyl group or an amino acid group.

In addition, the cyclic compound may comprise at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group,

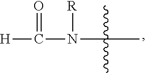

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², and R⁵ to R⁷ is identical or different and represents a C1-C5 alkyl group or C6-C12 aryl group:

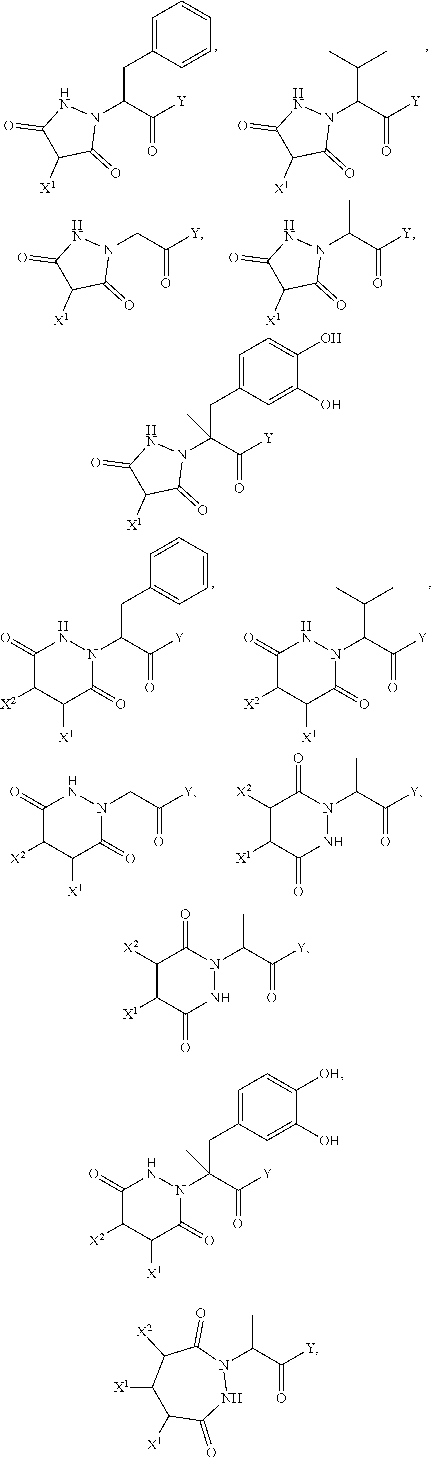

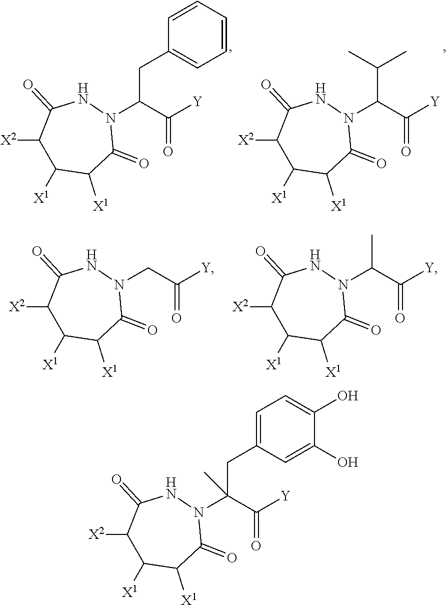

In addition, the cyclic compound may comprise at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group,

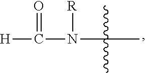

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², and R⁵ to R⁷ is identical or different and represents a C1-C5 alkyl group or C6-C12 aryl group:

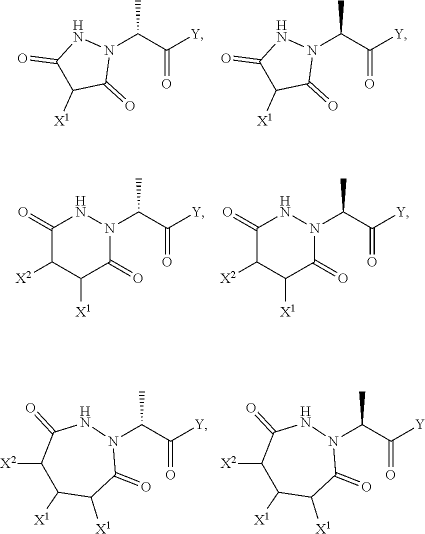

In addition, the cyclic compound may comprise at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group,

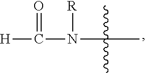

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², and R⁵ to R⁷ is identical or different and represents a C1-C5 alkyl group or C6-C12 aryl group:

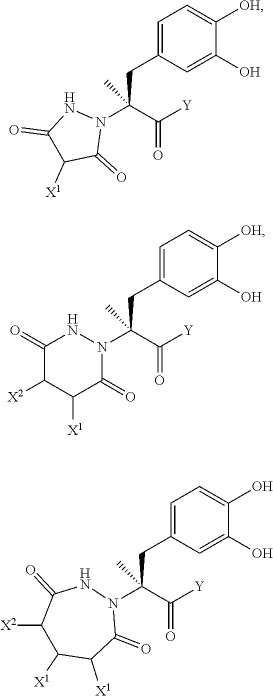

According to another aspect of the present invention, there is provided a method for cyclizing an unnatural substrate comprising a step of conjugating an acylated tRNA represented by Structural Formula 1 and an acylated tRNA represented by Structural Formula 2 in a translation reaction to produce a cyclic compound represented by structural formula 3.

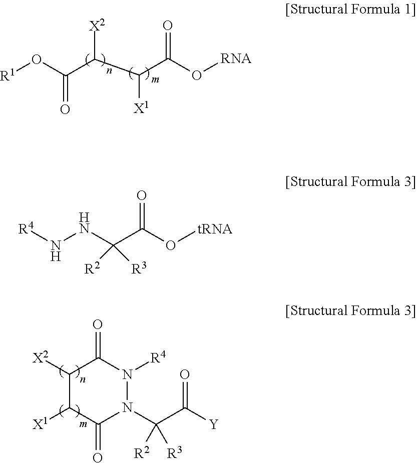

wherein R′ is a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group, or a substituted or unsubstituted (C6-C14 aryl) (C1-C8 alkyl) group,
X¹ and X² are each independently a hydrogen atom, a substituted or unsubstituted C1-C10 alkyl group, a substituted or unsubstituted C3-C7 cyclic alkyl group,

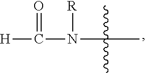

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the R¹, X¹ and X², R⁴ and R⁵ to R⁷ is identical or different and represents a C1-C5 alkyl group or C6-C12 aryl group, and a group suitable for substitution on the R² and R³ is identical or different and represents a C1-C5 alkyl group, C6-C12 aryl group, a hydroxy group or an amino acid group.

The cell-free protein synthesis system of the present invention can obtain a peptide having a highly efficient cyclic motif without going through a complicated chemical process by using an in vitro protein translation system.

In addition, the cyclic motif of the protein synthesized in the cell-free protein synthesis system of the present invention can exhibit higher pharmacological activity than a peptide including a simple non-natural substrate.

In addition, the cyclic motif of the protein synthesized in the cell-free protein synthesis system of the present invention has a high chemical stability because it contains a hydrazinedione group, so it is not easily decomposed even in cells and can maintain its structure for a long time

### BRIEF DESCRIPTION OF THE DRAWINGS

Since these drawings are for reference in explaining exemplary embodiments of the present invention, the technical idea of the present invention should not be interpreted as limited to the attached drawings.

FIG. 1 shows the new bond formation proposed in this work. The structure of the ribosome and tRNAs involved in bond formation from previously published work (PDB ID: 1vy4). Bifunctional substrates containing two electrophiles (E⁺) and nucleophiles (Nu⁻) engage in ring-closing reactions via successive aminolysis within the ribosome, resulting in the formation of cyclic structure (A, TOP). The typical ribosome-mediated peptide bond formation mechanism involving a nucleophilic substitution reaction (B, BOTTOM).

FIG. 2 shows peptide-based natural products with a cyclic motif. Cyclic motifs enhance the drug efficacy of peptides by providing stability against proteases and structural rigidity. Penicillin V, Patellamide C, Goadsporin, peptide with cyclic motif, are representative natural products with known high pharmacological activity.

FIG. 3A shows that each substrate was activated with a flexizyme leaving group (FLG), an essential motif for recognition by flexizyme (Fx). The diester substrates were synthesized from malonic and succinic acid, each containing two electrophiles but differing in number of carbon atoms in their structure. The substrates were further diversified by varying the R₁ and R₂ substituents. As synthons, the inventors designed a hydrazinoester containing two nucleophilic nitrogen atoms.

FIG. 3B shows that Fx was used to acylate the substrate onto in vitro transcribed synthetic tRNAs. Following the flexizyme-mediated acylation of diester and hydrazinoester, each acylated tRNA:ncM complex was added to a cell-free protein synthesis platform to decode two consecutive codons programmed on mRNA. The ribosome then forms non-standard 5—and 6-membered rings while polymerizing the two monomers, demonstrating its potential as a versatile chemical apparatus capable of facilitating novel synthetic reactions.

FIG. 4A shows diester substrates. the diester substrates were systemically designed based on the structure of malonic and succinic acid. Out of 21 (FIG. 7), 14 synthetic diesters substrates (1-12, m, and s) were successfully charged to tRNA by Fx, resulting in an ncM:tRNA^{fmet} (CAU) conjugate. Similarly, Hz was charged to tRNA by Fx, yielding Hz:tRNA^{Pro1E2}(GAU). Cyclization efficiency, measured as the ratio of peak intensities representing peptides with cyclic structures relative to linear peptide oligomers is depicted with shade intensity according to a color map.

FIG. 4B shows that upon introducing m:tRNA^{fmet} (CAU) and s:tRNA^{fmet} (CAU) with Hz:tRNA^{Pro1E2}(GAU) into PURExpress™ (NEB) system, the ribosome successfully synthesizes 5- and 6-membered ring structures, respectively.

FIG. 4C shows that mass spectrometric analysis of the reaction between substrates m and Hz within the ribosome produced a high yield (100%) of peptide oligomer with a cyclic pyrazolidinedione group at the N-terminus (upper panel).

FIG. 4D shows that however, the reaction between substrate s, which bears the same R₁ substituent, and Hz yielded only 20% cyclic structure alongside 80% linear product, indicating incomplete cyclization (lower panel). *Note that substrates 7-8 were synthesized as a mixture (see FIG. 14) and were used as such.

FIG. 5A shows that substrates 3 and 11 bearing a phenyl or benzyl substituent for R₁ were tested for cyclization. For malonate diester substrates, cyclization to form 5-membered rings was nearly complete (˜100% efficiency), indicating that the nature of R₁ has minimal influence on this process due to rapid ring-closure kinetics (upper panel). A notable enhancement in the formation of 6-membered rings was observed when R₁ was altered from methyl to phenyl, with no detectable linear peptide byproduct (lower panel).

FIG. 5B shows that the cyclization efficiency of 6-membered ring for succinate diester is considerably lower than that of 5-membered ring for malonate diester, when the R₁O⁻ substituent is ethoxylate (substrate 5) and methoxylate group (substrate 6, s, 7, and 8, see the table). The formation of 5-membered rings within the ribosome is kinetically most favorable, while the synthesis of 6-membered rings presents a challenge.

FIG. 5C shows that the challenge of 6-membered ring formation is lowered by the chemical properties of R₁O⁻ substituents. Phenoxylate with lower basicity (pKa of ˜10) enhances cyclization efficiency during the aminolysis reaction.

FIG. 6A shows two potential mechanisms are proposed for the cyclization process within the ribosome. In the first scenario, the α-nitrogen atom of Hz initiates an attack on the tRNA ester linkage, while the β-nitrogen atom targets the R₁ ester linkage, facilitating ring closure.

FIG. 6B shows the second scenario proposes the reverse order of attacks, leading to the formation of a regioisomeric cyclic structure.

FIG. 6C show the reaction of resulting linear peptide with 4-mba, a reagent that specifically reacts with free primary amines showed a single peak ([M+H]⁺=1258.4) corresponding to the mass of the linear product of a hydrazone. This is direct evidence that a free primary amine is present within the peptide, suggesting that the α-nitrogen was used to form the peptide bond and the β-nitrogen was used for ring-closure.

FIG. 7 shows synthetic non-canonical monomers (ncMs) used in this invention. The inventors synthesized 21 diesters and one hydrazino ester for this invention. The inventors evaluated diverse flexizyme leaving groups (cyanomethylester (CME), dinitrobenzylester (DNB), and 2—(aminoethyl) amidocarboxybenzyl thioester (ABT) ) to find optimal conditions for tRNA-charging. For example, substrate m was synthesized following the observation that m-2 was not effectively charged to tRNA. Pairs of substrates such as m, m-2, 7/7-2, and 11/11-2 share the same structure, but differ in their flexizyme leaving groups (FLGs). Despite our efforts, optimal Fx conditions for charging four of the substrates (0, 13, 14, and 15) were not determined.

FIG. 8 shows that acylation of microhelix (mihx) with malonate and hydrazinoesters. Acylation of microhelix with malonate substrates (m, m-2, 0, 1, 2, 3 and Hz): The Fx-catalyzed acylation reactions for six malonate diester and hydrazinoester substrates were carried by three different flexizymes (eFx, dFx, and aFx) at two different pH levels (7.5 and 8.8) over 16 h. The yield of each acylation reaction was determined by comparing the relative band intensity of unacylated and acylated mihx on the gel. The percent yields are 52.7% and 50.6% for substrate 3 and Hz. The yields for substrates 0, 1, m, m-2, and 2 could not be determined because the band shifts were unclear. Substrates that did not present clear band shifts were further validated via mass spectrometry. The arrow was used to indicate bands shifted due to an increase in size after acylation. The same reaction conditions were applied to tRNA acylation with the substrate.

FIG. 9 shows that acylation of microhelix with succinate ester. Acylation of microhelix with succinate (s, 4-15): The Fx-catalyzed acylation reactions for 15 succinate diester substrates were carried out with three different flexizymes (eFx, dFx, and aFx) at two different pH levels (7.5 and 8.8) over 16 h. The yield of each acylation reaction was determined by comparing the relative band intensity of unacylated and acylated mihx on the gel. The percent yields are 22.4%, 34.8%, 42.0%, 29.2%, 57.3%, 19.1% for substrate 4, 6, 8, 10, 11, 12. The yields for substrates 5, s, 7, 7-2, 9, 11-2, 13, 14, 15 could not be determined because the band shifts were unclear. Substrates that did not present clear band shifts were further validated via mass spectrometry.

The arrow was used to indicate bands shifted due to an increase in size increase after acylation. The same reaction conditions were applied to tRNA acylation with the substrate.

FIG. 10 shows confirmation of tRNA-charging using mass spectrometry analysis. For substrates (m, m-2, b, 0, 1, 2, 5, 7, 7-2, 9, 11-2, 13, 14, and 15) that did not show clear band shift on gel electrophoresis, the inventors further monitored acylation through mass spectrometric analysis. Each substrate was charged to tRNA^{fMet}(CAU) by Fx and supplemented into a PURExpress reaction, where the substrate:tRNA^{fMet}(CAU) conjugate is incorporated at the initiating AUG codon (position X) of a reporter peptide (Strep tag, XWSHPQFEK). Following the reaction, the peptide was purified using magnetic beads coated with strep-tactin antibody and characterized by MALDI mass spectrometry. For substrates m-2, 0, 7-2, 13, 14, and 15 that showed no peaks corresponding to the theoretical mass of XWHSPQFEK, the inventors confirmed that these were not charged to tRNA by Fx. Although band shifts on gel were unclear, substrates 1, m, 2, 5, s, 7, and 9 were found to be incorporated into a peptide (see FIGS. 7 and 8). Peaks marked as an asterisk (*) correspond to succinimide products resulting from side reactions of diesters.

FIG. 11 shows calculation of cyclization efficiency. The cyclization efficiency was determined as the ratio of the sum of peak areas of cyclized products to the sum of peak areas of linear and cyclized products. This assumes that peptides with similar amino acid sequences would be ionized and detected in proportion to their presence in the mixture.

FIG. 12 shows derivatives of cyclic hydrazinedione. The genetic code reprogramming approach enabled the formation of misacylated tRNA with synthetic ncMs. By altering the substrates, diverse pyrazolidinedione and tetrahydropyridazinedione derivatives were synthesized by the ribosome in vitro. Importantly, these reactions were carried out under mild conditions in aqueous solution (2 hours, 37° C.).

FIG. 13 shows ribosome-mediated synthesis of pyrazolidinedione derivatives. Characterization of the pyrazolidinedione products. Peaks marked with an alphabet (T) indicate a truncated peptide that lacks the first one or two ncMs at the N-terminus. All malonate diesters, regardless of R₁ and R₂, formed 5-membered cyclic pyrazolidinedione products.

FIG. 14 shows ribosome-mediated synthesis of tetrahydropyridazinedione derivatives. Characterization of tetrahydropyridazinedione products. Peaks marked with T indicate a truncated peptide that lacks the first one or two ncMs at the N-terminus. Succinate diesters, unlike malonate diesters, formed varying yields of the tetrahydropyridazinedione product depending on R₁. If R₁ was a good leaving group, a higher yield of tetrahydropyridazinedione products was detected. Peaks marked with L indicate linear products that did not undergo the cyclization reaction to 6-membered ring. Peaks marked with an asterisk (*) correspond to the theoretical mass of the peptide containing Ile at the AUC codon. This product was formed presumably because of contaminations of Ile in the PURExpress™ ΔtRNA, aa kit (NEB).

FIG. 15. Proposed mechanism of cyclic hydrazinedione formation within PTC of ribosome. (i) Nucleophilic attack of the α-amino group on the electrophilic carbonyl carbon in the ester linkage of the P-site tRNA leads to the formation of a zwitterionic tetrahedral intermediate, (ii) the deprotonation from the ammonium moiety results in oxonium formation at the 2′-hydroxy group (OH) of P-site tRNA, (iii) protonation of the 3′-OH group makes the P-site tRNA a good leaving group and transfers the nascent peptide into the A-site, (iv) a subsequent nucleophilic attack of the β-amino group on the electrophilic carbonyl carbon in the ester linkage of the R₁ group leads to the formation of a new zwitterionic tetrahedral intermediate, (v) deprotonation from the ammonium moiety by the 2′—OH group followed by alcohol cleavage and deprotonation from the 2′—OH group (vi) results in cyclic hydrazinedione formation.

FIGS. 16A to 16C show that in solution, the cyclization reaction does not occur through self-cyclization. The constant peak intensity of the linear product over time indicates that the cyclic products are not generated by a simple self-cyclization reaction. (See FIG. 17 for all the mass spectra obtained at the given time points).

FIG. 17 shows incubation of linear peptide. The full set of MALDI spectra used in FIG. 16A in the main text. The linear product bearing ncMs was incubated at 37° C. in Tris-HCl (pH 8.0) buffer and characterized using MALDI-TOF at 0.5, 1, 3, 5, 8, 12 and 24 h. There was no significant variation in cyclization efficiency over time, indicating that self-cyclization did not occur under the experimental conditions in solution.

FIGS. 18A to 18C show purification of linear peptide. Due to the possibility of self-cyclization during the purification process, the purification steps for the linear peptide containing ncMs was repeated. Then, linear peptide was characterized using MALDI-TOF. There was no significant variation in cyclization efficiency. The iterative (n=3) purification step requiring an antibody and heat does not increase the yield of cyclic product.

FIG. 19A shows that the P1c2 domain, which partially includes the ribosome's catalytic domain (domain V) was prepared via in vitro transcription.

FIG. 19B shows that P1c2 was designed to have a UGGU sequence, facilitating base-pairing with a tRNA with an ACCA sequence at the 3′-end.

FIG. 19C shows that upon dimerization, the P1c2 domain forms a catalytic pocket akin to the peptidyl transferase center. This arrangement allows the two tRNA-charged substrates to undergo cyclization. Subsequently, RNase digestion of the resulting tRNAs yields the expected product attached to the 3′-end residue of the tRNA.

FIG. 19D shows that the extracted ion chromatograms show a single peak corresponding to the theoretical mass of a 5-membered ring linked to the 3′-OH of adenosine. [M+H]⁺=422.141 (calculated), [M+H]⁺=422.145 (observed).

FIG. 19E shows that no peak corresponding to the theoretical mass of linear form was detected.

FIG. 20A shows that P1c2 UGGU allows the two tRNA-charged substrates to undergo dimerization in dihydrazinealanine formation through P1c2. As no substrates order is defined by mRNA, hydrazino alanine (Hz) reacts to itself, resulting in the formation of a dimer (DiHz).

FIG. 20B shows that The extracted ion chromatograms show a peak at 8.19 min corresponding to the theoretical mass of a DiHz linked to the 3′-OH of adenosine. [M+H]⁺=440.200 (cal), [M+H]⁺=440.206 (obs). This indicates that P1c2 indeed catalyzes the reaction of cyclization reaction of Hz with a diester substrate.

FIG. 21 shows a diagram showing the plasmid maps of pJL1_MI-StrepII (MIWSHPQFEK), pJL1_M-StrepII-ST (MWSHPQFEKST, and pJL1_MR-StrepII-DR (MRWSHPQFEKDR).

FIG. 22 shows a diagram showing the symbols, steps, primer names, and gene sequences of the primers.

FIG. 23 shows MALDI spectrum of the (1)-Hz-StrepII-(1)-Hz and (12)-Hz-StrepII-(12)-Hz peptide. The genetic template was designed to consecutively incorporate the monomers in an alternating fashion, so the resulting peptide contains multiple cyclic backbones. The resulting peptides were purified and characterized. Calculated mass: [M+H]⁺=1423.6 and [M+H]⁺=1452.6 for 5—and 6-membered ring-formation reaction, respectively.

### DESCRIPTION OF THE PREFERRED EMBODIMENTS

Hereinafter, embodiments of the present invention will be described in detail with reference to the attached drawings so that those skilled in the art can easily implement the present invention.

However, the following description is not intended to limit the present invention to specific embodiments, and in describing the present invention, if it is determined that a detailed description of related known technology may obscure the gist of the present invention, the detailed description will be omitted.

Terms used herein are merely used to describe specific embodiments. This is not intended to limit the present disclosure. Singular expressions include plural expressions unless the context clearly dictates otherwise. In this application, terms such as “comprise” or “have” are intended to indicate the presence of features, numbers, steps, operations, components, or a combination thereof described in the specification. Accordingly, the term should be understood as not excluding in advance the presence or addition of one or more other features, numbers, steps, operations, components, or combinations thereof.

Hereinafter, acylated tRNA, introduction of pharmacologically active motif in peptide using ribosome catalysis and method of preparing same will be described in detail. However, this is presented as embodiments, and the present invention is not limited thereby, and the present invention is only defined by the scope of the claims to be described later.

According to one aspect of the present invention, there is provided an acylated tRNA represented by Structural Formula 1 or Structural Formula 2 below:

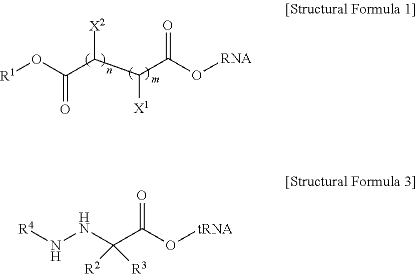

wherein R′ is a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group, or a substituted or unsubstituted (C6-C14 aryl) (C1-C8 alkyl) group,
X¹ and X² are each independently a hydrogen atom, a substituted or unsubstituted C1-C10 alkyl group, a substituted or unsubstituted C3-C7 cyclic alkyl group,

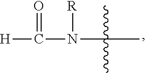

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the R¹, X¹ and X², and R⁴ is identical or different and represents a C1-C5 alkyl group or C6-C12 aryl group, and a group suitable for substitution on the R² and R³ is identical or different and represents a C1-C5 alkyl group, C6-C12 aryl group, a hydroxyl group or an amino acid group.

In addition, the acylated tRNA may be represented by the Structural Formula 1 and may comprise at least one selected from the group consisting of compounds below:

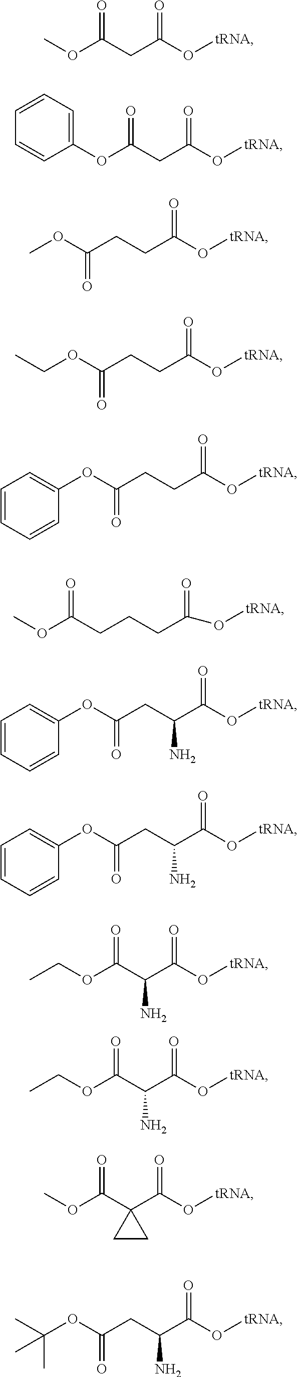

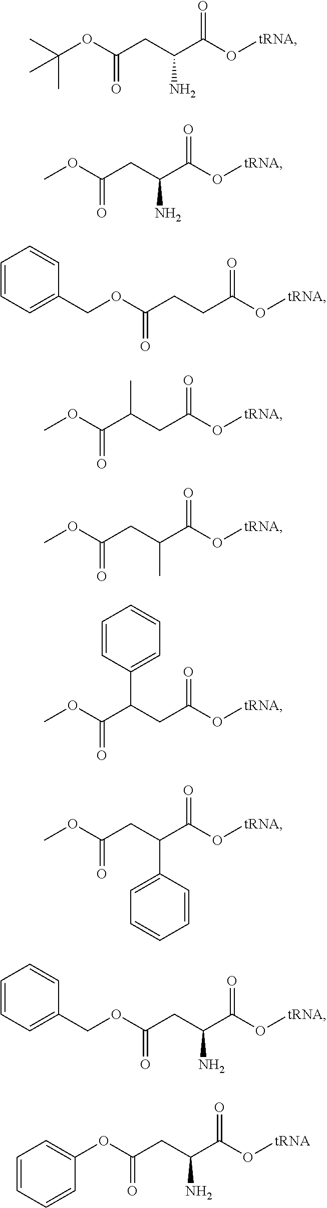

In addition, the acylated tRNA may be represented by the Structural Formula 2 and may comprise at least one selected from the group consisting of compounds below:

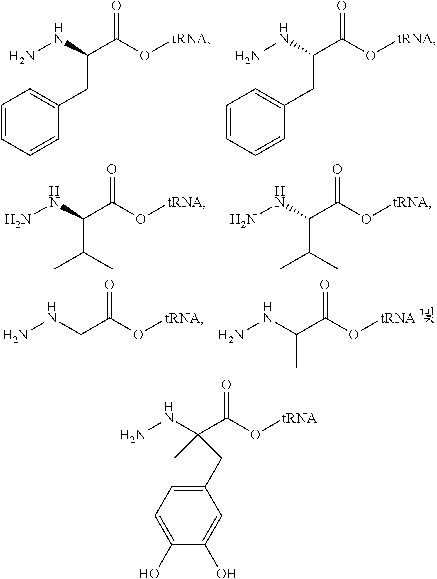

In addition, the acylated tRNA may represented by the Structural Formula 2 and comprises at least one selected from the group consisting of compounds below:

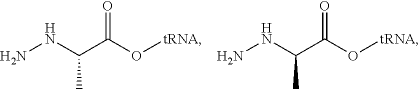

In addition, the acylated tRNA may be represented by the Structural Formula 2 and may comprises at least one selected from the group consisting of compounds below:

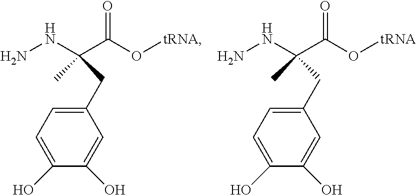

According to another aspect of the present invention, there is provided a cyclic compound represented by structural formula 3 below:

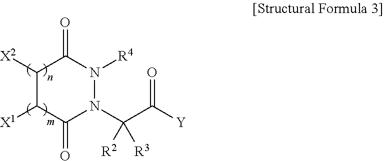

wherein X¹ and X² are each independently a hydrogen atom, a substituted or unsubstituted C1-C10 alkyl group, a substituted or unsubstituted C3-C7 cyclic alkyl group,

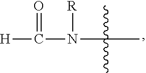

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
Y is a hydrogen atom, -O (tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², R⁴ and R⁵ to R⁷ is identical or different and represents a C1-C5 alkyl group or C6-C12 aryl group, and a group suitable for substitution on the R² and R³ is identical or different and represents a C1-C5 alkyl group, C6-C12 aryl group, a hydroxyl group or an amino acid group.

In addition, the cyclic compound may comprise at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group,

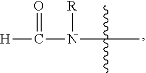

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², and R⁵ to R⁷ is identical or different and represents a C1-C5 alkyl group or C6-C12 aryl group:

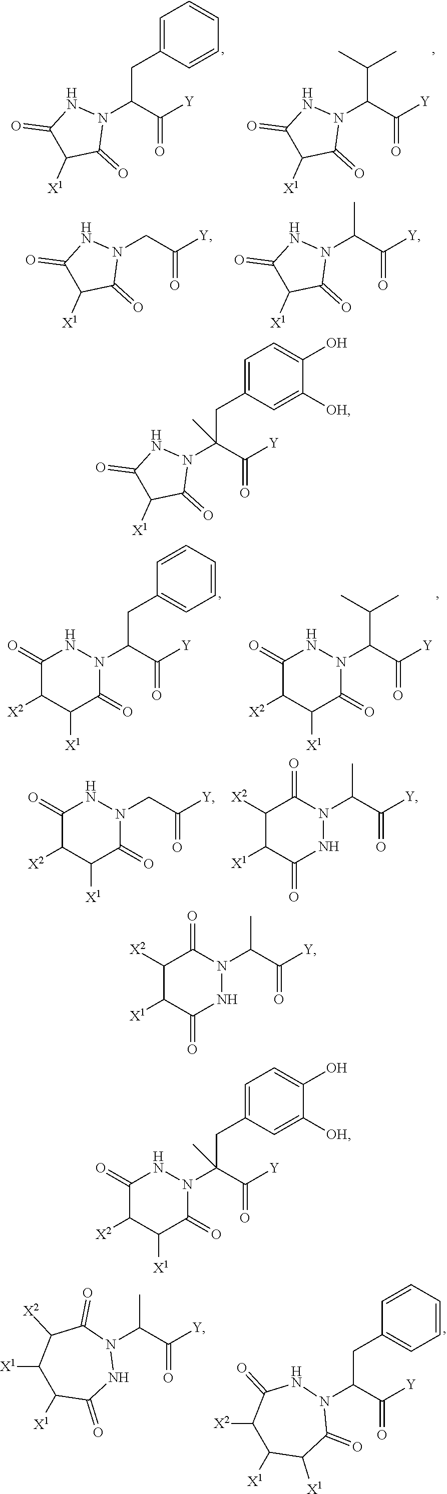

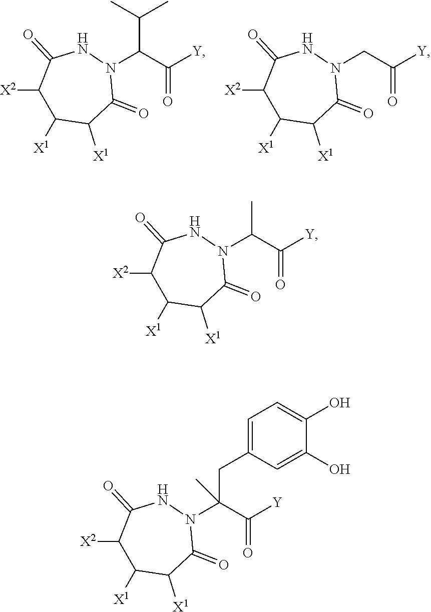

In addition, the cyclic compound may comprise at least one selected from the group consisting of compounds below,
wherein X and X are each independently a hydrogen atom, a substituted or unsubstituted C1-C10 alkyl group, a substituted or unsubstituted C3-C7 cyclic alkyl group,

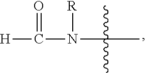

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², and R⁵ to R⁷ is identical or different and represents a C1-C5 alkyl group or C6-C12 aryl group:

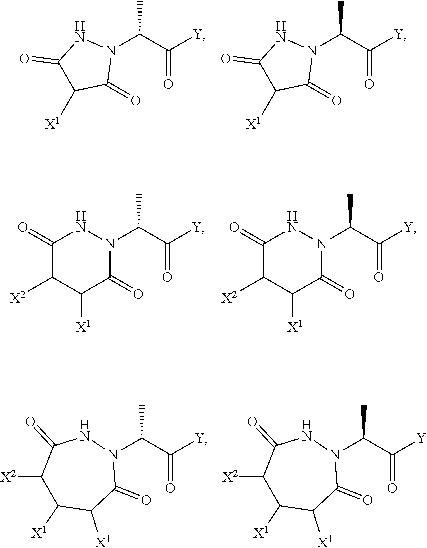

In addition, the cyclic compound may comprise at least one selected from the group consisting of compounds below,
wherein X¹ and X² are each independently a hydrogen atom, a substituted or unsubstituted C1-C14 alkyl group, a substituted or unsubstituted C3-C7 cyclic alkyl group,

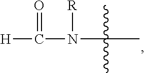

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the X¹ and X², and R⁵ to R⁷ is identical or different and represents a C1-C5 alkyl group or C6-C12 aryl group:

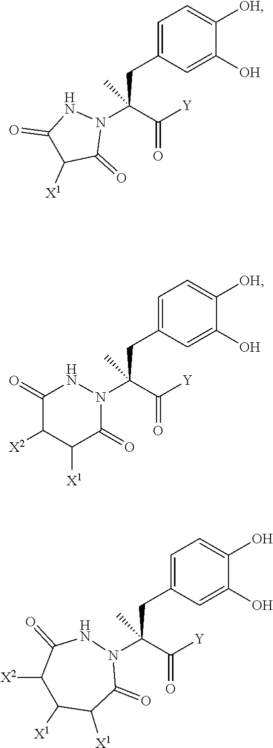

According to another aspect of the present invention, there is provided a method for cyclizing an unnatural substrate comprising a step of conjugating an acylated tRNA represented by Structural Formula 1 and an acylated tRNA represented by Structural Formula 2 in a translation reaction to produce a cyclic compound represented by structural formula 3.

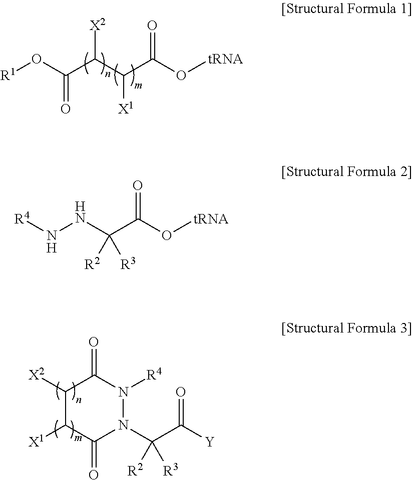

wherein R¹ is a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group, or a substituted or unsubstituted (C6-C14 aryl) (C1-C8 alkyl) group,
X¹ and X² are each independently a hydrogen atom, a substituted or unsubstituted C1-C10 alkyl group, a substituted or unsubstituted C3-C7 cyclic alkyl group,

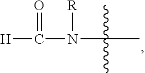

a substituted or unsubstituted C6-C14 aryl group, a substituted or unsubstituted (C1-C8 alkyl) (C6-C14 aryl) group, a substituted or unsubstituted (C6-C14 aryl) (C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group,
Y is a hydrogen atom, -O(tRNA), -0(R⁵), —N(R⁶) (R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl) (C6-C14 aryl) group or a substituted or unsubstituted (C6-C14 aryl) (C1-C8 alkyl) group, and
a group suitable for substitution on the R¹, X¹ and X², R⁴ and R⁵ to R⁷ is identical or different and represents a C1-C5 alkyl group or C6-C12 aryl group, and a group suitable for substitution on the R² and R³ is identical or different and represents a C1-C5 alkyl group, C6-C12 aryl group, a hydroxy group or an amino acid group.

### EXAMPLES

Hereinafter, the present invention will be described with reference to preferred embodiments. However, this is for illustrative purposes only and does not limit the scope of the present invention.

All materials were of the best grade commercially available and used without further purification. NMR solvents (DMSO-d₆) were purchased from Sigma-Aldrich or TCI Chemicals. All the oligonucleotides used in this research were purchased from Integrated DNA Technologies (IDT), Bioneer, or Cosmogenetech and used as received according to manufacturer's guidelines.

General synthesis of substrates Preparation of diesters. The diester substrates were synthesized in the following ways: i) The inventors utilized a commercially available molecule containing a carboxylic acid and an ester substituted with R₁ as a starting material or an anhydride that would yield an R₁ ester upon hydrolysis, ii) Following the formulation of diverse R₁ substituents, the inventors carried out esterification reactions to attach FLG for Fx-charging.

Preparation of hydrazine substrate. The hydrazine substrate was synthesized using a methyl lactate in four steps: i) replacement of hydroxyl to hydrazino group (triflation on the hydroxyl group followed by attachment of Boc-NHNH₂), ii) conversion of ester to acid by base-catalyzed hydrolysis, iii) attachment of Boc-ABT, and iv) Boc deprotection from the β-nitrogen and ABT.

### Preparation of activated substrates for flexizyme reaction

General synthetic procedure A: Formation of cyanomethyl ester. To a solution of carboxylic acid (1 eq) triethylamine (0.5 eq) and bromoacetonitrile (10 ml) were added and stirred overnight. After stirring for 16 h at room temperature, the reaction mixture was diluted with EtOAc and washed with HCl (1.0 M in water), NaHCO₃ (4% (w/v) in water), brine, and dried over MgSO₄. The organic phase was concentrated to provide the crude product.

General synthetic procedure B: Formation of dinitrobenzyl esters. To a solution of carboxylic acid (1 eq), dinitrobenzyl alcohol (1.2 eq), 4-dimethylaminopyridine (DMAP) (0.5 eq) and N-(3-dimethylaminopropyl) -N′-ethylcarbodiimide hydrochloride (EDC.HCl) (1.2 eq) were added in dichloromethane. After stirring for 16 h at room temperature, the reaction mixture was diluted with EtOAc and washed with HCl (1.0 M in water), NaHCO₃ (4% (w/v) in water), brine, and dried over MgSO₄. The organic phase was concentrated to provide the crude product.

General synthetic procedure C: Formation of 4-((2-aminoethyl)carbamoyl)benzyl thioates. To a solution of carboxylic acid (1.0 eq), tert-butyl 2-[4-(mercaptomethyl)benzamido]ethyl carbamate (Boc-ABT) (0.3 eq), 4-dimethylaminopyridine (DMAP) (0.5 eq) and N-(3-dimethylaminopropyl) -N′-ethylcarbodiimide hydrochloride (EDC.HCl) (1.0 eq) were added in DCM. After stirring for 16 h at room temperature, the reaction mixture was diluted with EtOAc and washed with HCl (1.0M in water), NaHCO₃ (4% (w/v) in water), brine, and dried over MgSO₄. The organic phase was concentrated to provide the crude product.

### Plasmid Production

The pJL1 plasmid variants used were prepared as follows using pJL1 (https://www.addgene.org/69496/) shared on Addgene. Primers were designed to have the sequences of pJL1_M-StrepII-ST (MWSHPQFEKST), pJL1 MI-StrepII (MIWSHPQFEK), and pJL1 MR-StrepII-DR (MRWSHPQFEKDR) based on pJL1_M-StrepII (MWSHPQFEK), and the primers were ordered through Bioneer and Cosmogenetech. The ordered primers were dissolved to 100 mg/μl, mixed with the existing pJL1_M-StrepII, and PCR was performed. DNA fragments obtained through PCR were separated into DNA of the desired size through electrophoresis, and then T5 nuclease was added to perform Gibson assembly to anneal matching sequences, and plasmids were connected using polymerase and ligase. Since pJL1 contains a kanamycin resistance gene, the synthesized plasmid was transformed into E. coli and cultured in LB mixed with kanamycin to selectively amplify the plasmid. The base sequence of the manufactured plasmid was confirmed by Sanger sequencing or NGS.

### General Fx-Mediated Acylation Reaction

Microhelix acylation. 1 μL of 0.5 M HEPES (pH 7.5) or bicine (pH 8.8), 1 μL of 10 μM microhelix, and 3 μL of nuclease-free water were mixed in a PCR tube with 1 μL of 10 μM eFx, dFx, and aFx, respectively. The mixture was heated for 2 min at 95° C. and cooled down to room temperature over 5 min. 2 μL of 300 mM MgCl₂ was added to the cooled mixture and incubated for 5 min at room temperature. Followed by the incubation of the reaction mixture on ice for 2 min, 2 μL of 25 mM activated ester substrate in DMSO was then added to the reaction mixture. The reaction mixture was further incubated for 16-120 h on ice in a cold room at 4° C.

tRNA acylation. 2 μL of 0.5 M HEPES (pH 7.5), 2 μL of 250 μM tRNA, 2 μL of 250 μM of a Fx selected from the respective microhelix experiment and 6 μL of nuclease-free water were mixed in a PCR tube. The mixture was heated for 2 min at 95° C. and cooled down to room temperature over 5 min. 4 μL of 300 mM MgCl₂ was added to the cooled mixture and incubated for 5 min at room temperature. Followed by the incubation of the reaction mixture on ice for 2 min, 4 μL of 25 mM activated ester substrate in DMSO was then added to the reaction mixture. The reaction mixture was further incubated for the optimal time determined during the microhelix experiment on ice in cold room.

In vitro synthesis of cyclic hydrazinediones

Ribosome-mediated synthesis of cyclic hydrazinediones. As a reporter peptide, a T7 promoter-controlled DNA template (pJL1_XZ-StrepII) was designed to encode a streptavidin (Strep) tag and additional Met(AUG) and Ile(AUC) codons (XZWHSPQFEKST (strep-tag), where X and Z indicate the position of the diester and Hz substrates, respectively) for N-terminal incorporation of two ncMs. The synthesis was performed using only the 9 amino acids that decode the purification tag without the other 11 amino acids to prevent corresponding endogenous tRNAs from being aminoacylated and participating in translation. The PURExpress™ A (aa, tRNA) kit (NEB, E6840S) was used for in vitro ribosomal synthesis and the reaction mixtures were incubated at 37° C. for 2 h. The synthesized peptides were then purified using Strep-Tactin®-coated magnetic beads (IBA), denatured with a 0.1% (v/v) SDS solution in water, and characterized by MALDI-TOF mass spectrometry.

Multiple syntheses of cyclic hydrazinediones. A new plasmid (pJL1-StrepII-2XZYZ) encoding the XZWHSPQFEKSX′Z was used for multiple ring-closing reactions, where X, X′, and Z indicate the position of the diester (1, 12) and Hz, respectively. The diesters were incorporated into the Met (AUG-X) and Asp (GAC-X′) codons. Simultaneously, Hz was incorporated into the Arg (CGU-Z) codon. The reaction mixtures, containing only the 8 amino acids necessary for decoding the Strep tag, were incubated at 37° C. for 2 hours. The reaction mixtures were incubated at 37° C. for 2 h. The resulting products were purified and characterized by the same method described below.

Purification and characterization of cyclic hydrazinediones

The products containing hydrazinediones were purified using an affinity tag purification technique or a C18 spin column purification. The purified products were characterized by MALDI spectrometry. For MALDI sample preparation, the purified peptide in 1.5 μL of SDS solution was dried with 0.5 μL of the matrix (α-cyano-4-hydroxycinnamic acid in THF, 10 mg·mL⁻¹). The dried sample was characterized on a Bruker Autoflex MALDI-TOF and processed using Compass DataAnalysis (Bruker).

Reaction of hydrazone between hydrazine and aldehyde

The sample was freeze-dried to remove a solvent (water). Then, the solid-state peptide was dissolved by 4-methylbenzaldehyde (TCI, T0259) and incubated for 30 min at 37° C. Afterward, water was added again, and if layer separation occurs, the upper aqueous layer was characterized by MALDI-TOF and processed using Compass DataAnalysis (Bruker).

Reaction of pyrazolidinedione using ribozyme:P1c2^{UGGU}

1000 pmol of P1c2UGGU and was dissolved in 32 μL of 62.5 mM HEPES (pH 7.5). 8 μL of 300 mM MgCl₂ was added to the cooled mixture and incubated on ice for 60 min to be self-dimerized. Then, 1000 pmol of tRNA^{fMet} (CAU): (s) and tRNA^{Pro1E2} (GGU): Hz was added and placed at 4° C. for 24 h. The reaction product was treated with 44 μl RNase A solution (RNase A (Favorgen) and 200 mM sodium acetate, pH5.2) to liberate any pyrazolidinedione that might form and incubated for 5 min at room temperature. The treated sample was then transferred to autosampler vials, kept on ice until immediately before LC-MS analysis and returned to ice immediately afterwards. As a negative control, P1c2 was not added to the buffer with the same composition in the first step.

### Methods and Materials

All chemicals were purchased from Sigma-Aldrich, Sejin TCI, and Alpha Aesar, and used as received without further purification. Synthetic DNA oligonucleotides were purchased from IDT Korea, Bioneer (Korea), and Cosmogenetech (Korea). Silica gel flash chromatography was performed using 0.040-0.063 mm, 60 Å silica purchased from Supelco®. Thin layer chromatography was performed using glass silica plates coated with fluorescent indicator (F254) purchased from Supelco®. Sand, sodium chloride, sodium bicarbonate, potassium carbonate, concentrated hydrochloric acid, and sodium hydroxide pellets were purchased from Sigma-Aldrich, Biosesang (Korea), Samchun (Korea) and Daejung (Korea).

MALDI Mass spectra were recorded on a Bruker Autoflex. High-resolution mass spectrometry (HRMS) analysis was performed at the Department of Chemistry at Pohang University of Science and Technology (POSTECH) using the 6560 Accurate Mass Q-TOF LC/MS system from Agilent Technologies. MALDI mass spectra were processed using Compass DataAnalysis Viewer v6.1 (Bruker) and FlexControl v2.0 (Bruker) software utilizing the smoothening and baseline subtraction. 1H and 13C NMR spectra were collected either using a Bruker AVANCE III™ HD 500 MHz cryoprobe or Bruker AVANCE III™ HD 400 MHz cryoprobe NMR spectrometer and processed with TopSpin (4.3.0). Chemical shifts, denoted in ppm, were assigned relative to the residual NMR solvent peaks.

### Synthesis RNAs (tRNA, Fxs, and P1c2

DNA template synthesisExtension: 2.5 μL of 10 μM forward and reverse primer (Ext.) (see List of primers) was added to 10 μL KAPA HiFi 2× polymerase, and mixed with 5 μL dH2O in a PCR tube. The thermocycling conditions were as follows: 5 min at 95° C. followed 37° C. for 10 min and 72° C. for 30 s. PCR amplification: 20 μL of the product was used as an extension template, 20 μL each of 10 μM forward and reverse primer (Amp.) (see List of primers) were added to 250 μL KAPA HiFi 2× polymerase, and mixed with 190 μL dH2O in a PCR tube. The mixture was divided into several PCR tubes, and the DNA was amplified by the following thermocycling conditions: 3 min at 95° C., followed by 25 cycles of 95° C. for 30 s and Tm (either 55° C. or 65° C.) for 30 s, and 72° C. for 1 min. PCR products were assayed on a 1% (w/v) agarose gel.

DNA template precipitation

PCR products were extracted using phenol/chloroform/isoamyl alcohol and precipitated with ethanol. Samples were dried at room temperature for 5 min and resuspended in 100 μL nuclease-free water. DNA concentrations were determined from absorbance measured on a Nabi UV/Vis Nano Spectrophotometer. (MicroDigital Co., Ltd)

In vitro transcription

RNAs were prepared using a HiScribe® T7 quick high-yield RNA synthesis kit (NEB, E2050S). For in vitro transcription, 25 μg of DNA template was used with 50 μL of T7 RNA polymerase mix, 250 μL of NTP buffer mix, and nuclease-free water up to 500 μL. The mixture was incubated at 37° C. overnight.

Digestion of DNA templates

The DNA templates were removed by adding 50 μL of DNase I (NEB, E2050S) into the 500 μL of transcription reaction products. The reaction mixture was incubated at 4° C. for 2 h.

Purification of in vitro transcribed RNA

The digested transcription reactions were mixed with 500 μL 2× RNA loading dye and loaded onto a 15% TBE-Urea gel (24 g of Urea, 18.75 mL of 40% Acrylamide/Bis solution 19:1 (Bio-Rad, #1610144), and 5 mL of 3 M NaOAc, 10× TBE buffer up to 50 mL using water). The gel was run in 0.5× TBE buffer at 130V for 3 h at room temperature. The gel was placed on a silica plate and the transcribed RNAs were visualized by irradiation via a UV lamp (260 nm). The RNA products were excised from the gel and added to 5 mL of water. The gels were crushed and then placed in the cold room for 1 h. The gels were transferred to a HiGene™ HiFilter with a collection tube (Biofact) and centrifuged at 4000×g for 2 min. The flow-through was collected and added to the solution of 0.3 mL of 5M NaCl and 10 mL of 100% ethanol. The solution was placed at −20° C. for 16 h and centrifuged at 13,000×g for 30 min at 4° C. The supernatant was removed, and the pellet was dried for 5 min at room temperature. The dried RNA pellet was dissolved in nuclease-free water and the concentration was determined using a spectrophotometer.

Gel electrophoresis

### Preparation of acidic gel (pH 5.2)

10.8g of UREA, 15 mL of 40% Acrylamide/Bis Solution 19:1 (Bio-Rad, #1610144), and 500 μL of 3 M NaOAc were mixed in a 50 mL tube. Water added to a total volume of 30 mL. After the urea was completely dissolved, 300 μL of 10% (wt/vol) APS and 24 μL of TEMED were added to the solution. The mixture solution was casted into a gel cassette immediately using a serological pipette and the gel was allowed to polymerize overnight.

### Assaying optimal Fx-charging conditions

1 μL of prepared sample was mixed with 5 μL of acidic-loading dye (3 M NaOAc (pH 5.2), 20 μL of 0.5 M EDTA (pH 8.0), 980 μL of 2% (wt/vol) formamide, and 8 μL of Bromophenol Blue) and loaded onto a prepared 10% acidic urea gel. The gel was run in 50 mM NaOAc buffer at 135V for 2 h at 4° C. in a cold room. After electrophoresis, gel was stained by 2.5 uL of SYBR™ Gold Nucleic Acid Gel Stain (Invitrogen, S11494) in 50 mL of water for 10 min.

The gel was visualized on a Bio-Rad gel documentation system (GelDoc Go).

### Preparation Example

### Synthesis non-canonical monomers (ncMs)

### Synthesis of malonate diester

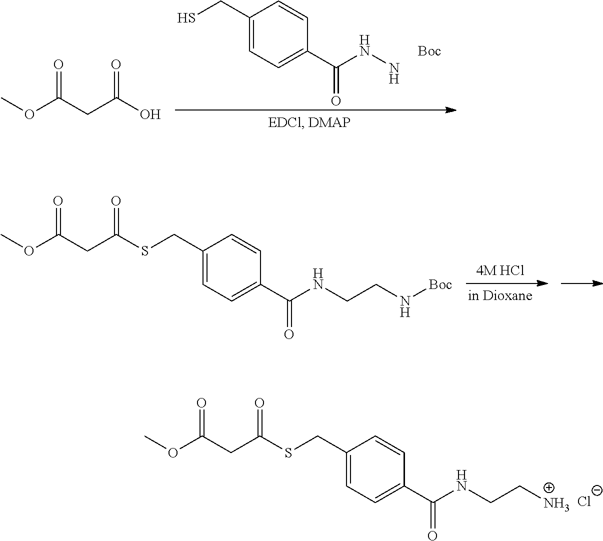

Methyl 3-((4-((2-aminoethyl)carbamoyl)benzyl)thio)-3-oxopropanoate (m): Prepared according to the general procedure A using methyl hydrogen malonate (341.1 μl, 3 mmol), Boc-ABT (155.2 mg, 0.5 mmol), DMAP (61.1 mg, 0.5 mmol), EDC-HCl (575.1 mg, 3 mmol) and CH₂Cl₂. (10 mL). Purification by flash column chromatography (60% EtOAc in n-Hex) yielded the Boc-protected product as a white solid (82.9 mg, 40.4%). The deprotection was achieved upon treatment with 4 M solution of HCl in 1,4-dioxane for 30 min at room temperature, and the resulting product was used without further purification. : ¹H NMR (500 MHz, DMSO-d₆) δ 8.72 (t, J=5.28 Hz, 1H), 8.01 (br s, 3H), 7.85 (d, J=8.19 Hz 2H), 7.41 (d, J=8.26 Hz, 2H),4.22 (s, 2H), 3.84 (s, 2H), 3.64 (s, 3H), 3.51 (q, J=5.90 Hz, 2H), 2.98 (q, J=5.69 Hz, 2H), 13C NMR (126 MHz, DMSO-d₆) δ 191.4 (1C, O═C—S), 166.9 (1C, O═C—O), 166.9 (1C, O═C—NH), 141.3 (1C, Ph), 133.4 (1C, Ph), 129. 1 (2C, Ph), 128.1 (2C, Ph), 52.7 (1C, H₃C—O), 49.1 (1C, CH₂), 39.1 (1C, CH₂NH₃⁺), 37.5 (1C, NHCH₂), 32.8 (1C, SCH₂). HRMS (ESI): Exact mass calc. for C₁₄H₁₉N₂O₄S+[M+H]⁺=311.1060, ; Found 311.1089.

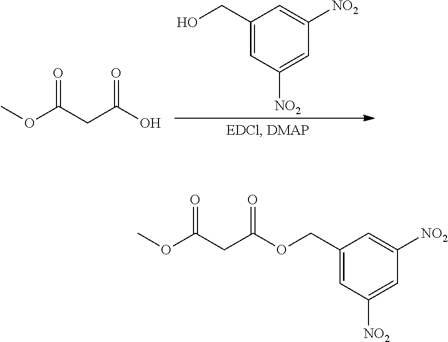

3,5-dinitrobenzyl methyl malonate (m-2) Prepared according to the general procedure B using methyl hydrogen malonate (104.7 μL, 1 mmol), 3,4-dinitrobenzyl alcohol (198.1 mg, 1 mmol), DMAP (122.2 mg, 1 mmol), EDC-HCl (191.7 mg, 1 mmol) and CH₂Cl₂ (10 ml). Purification by silica gel flash chromatography (30% EtOAc in n-Hex) yielded a yellow solid. (46.9 mg, 15.7%): ¹H NMR (500 MHz, DMSO-d₆) δ 8.80 (t, J=2.20 Hz, 1H), 8.67 (d, J=1.84 Hz, 2H), 5.43 (s, 2H), 3.69 (s, 2H), 3.68 (s, 3H), ¹³C NMR (126 MHz, DMSO-d₆) δ 169.3 (1C, O═C), 168.7 (1C, O═C), 150.7 (2C, C—N(═O)═O), 142.7 (1C, Ph), 130.4 (2C, Ph), 120.5 (1C, Ph), 66.6 (1C, OCH₂), 54.8 (1C, O—CH₃), 43.1 (1C, CH₂, Overlapping Solvent peak). HRMS (ESI): Exact mass cal. for C₁₁H₁₀N₂O₈Na⁺[M+Na]⁺=321.0329; Found 321.0321.

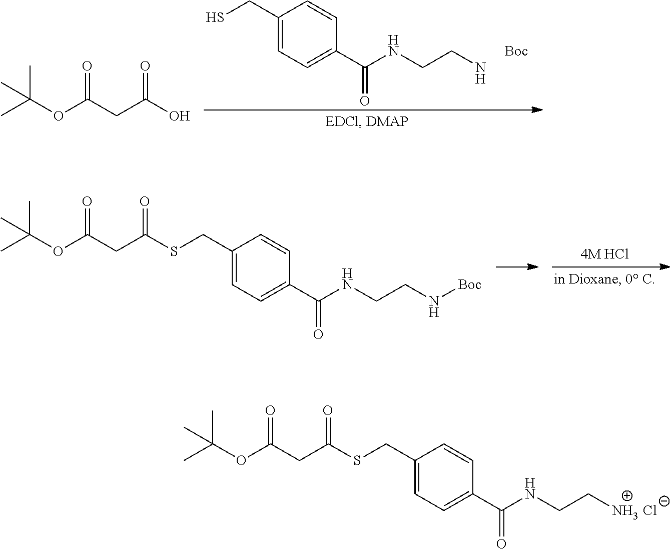

tert-butyl 3-((4-((2-aminoethyl)carbamoyl)benzyl)thio)-3-oxopropanoate (0): Prepared according to the general procedure A using mono-tert-butyl malonate (710.0 mg, 5 mmol), Boc-ABT (310.4 mg, 1 mmol), DMAP (122.2 mg, 1 mmol), EDC-HCl (383.4 mg, 2 mmol) and CH₂Cl₂. (10 mL). Purification by flash column chromatography (30% EtOAc in n-Hex) yielded Boc-protected product as a white solid. The deprotection was achieved upon treatment with 4 M solution of HCl in 1,4-dioxane at 0° C. for 30 min, and the resulting product was used without further purification. The product was obtained as a white solid. (32.8 mg, 8.4%). : ¹H NMR (500 MHz, DMSO-d₆) δ 8.79 (t, J=4.94 Hz, 1H), 8.15 (br s, 3H), 7.94-7.84 (m, 2H), 7.43 (d, J=8.03 Hz, 2H), 4.22 (s, 2H), 3.69 (s, 2H), 3.56-3.49 (m, 2H), 2.99-2.93 (m, 2H), 1.39 (s, 9H)¹³C NMR (126 MHz, DMSO-d₆) δ 191.6 (1C, O═C—S), 166.9 (1C, O═C—O), 165.6 (1C, O═C—NH), 141.4 (1C, Ph), 133.3 (1C, Ph), 129.0 (2C, Ph), 128.1 (2C, Ph), 82.0 (1C, C—O), 50.8 (1C, CH₂), 39.0 (1C, CH₂NH₃⁺), 37.6 (1C, NHCH₂), 32.7 (1C, SCH₂), 28.0 (3C, (CH₃)₃). HRMS (ESI): Exact mass cal. for C₁₇H₂₅N₂O₄S⁺[M+H]⁺=353.1530; Found 353.1555.

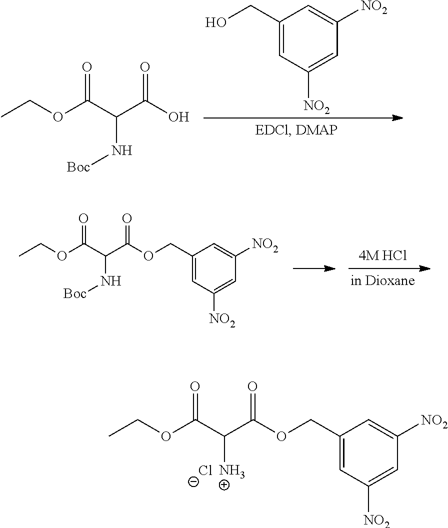 [SMILES: CCOC(=O)C(NC(=O)OC(C)(C)C)C(=O)O ; O=[N+]([O-])c1cc(CO)cc([N+](=O)[O-])c1 ; CCOC(=O)C(NC(=O)OC(C)(C)C)C(=O)OCc1cc([N+](=O)[O-])cc([N+](=O)[O-])c1 ; CCOC(=O)C([NH3+])C(=O)OCc1cc([N+](=O)[O-])cc([N+](=O)[O-])c1 ; [Cl-]]

1-(3,5-dinitrobenzyl) 3-ethyl 2-aminomalonate (1) Prepared according to the general procedure B using 2-((tert-butoxycarbonyl) amino)-3-ethoxy-3-oxopropanoic acid (100 mg, 0.4 mmol), 3,4-dinitrobenzyl alcohol (198.13 mg, 1 mmol), DMAP (122.2 mg, 1 mmol), EDC-HCl (191.7 mg, 1 mmol) and CH₂Cl₂ (10 mL). The product was successfully obtained. The deprotection was achieved upon treatment with 4 M solution of HCl in 1,4-dioxane for 30 min at room temperature, and the resulting product was washed with diethyl ether to remove 3,4-dinitrobenzyl alcohol. The solvent was concentrated under vacuum, and the product was obtained as a yellow solid (163.6 mg, 45.0%). : ¹H NMR (500 MHz, DMSO-d₆) δ 9.23 (br s, 3H), 8.84 (t, J=2.21 Hz, 1H), 8.74 (d, J=2.10 Hz, 2H), 5.58 (q, J=16.03 Hz, 2H), 5.26 (s, 1H), 3.56 (s, 2H), 1.22 (t, J=7.08 Hz, 3H)¹³C NMR (126 MHz, DMSO-d₆) δ 164.0 (2C, O═C), 148.5 (2C, C—N(═O)═O), 139.7 (1C, Ph), 128.7 (2C, Ph), 119.0 (1C, Ph), 66.8 (1C, O—CH₂-Ph), 66.2 (1C, CH—NH₃⁺), 63.6 (1C, OCH₂), 14.2 (1C, CH₃). HRMS (ESI): Exact mass cal. for C₁₂H₁₄N₃O₈⁺[M+H]⁺=328.0775; Found 328.0803.

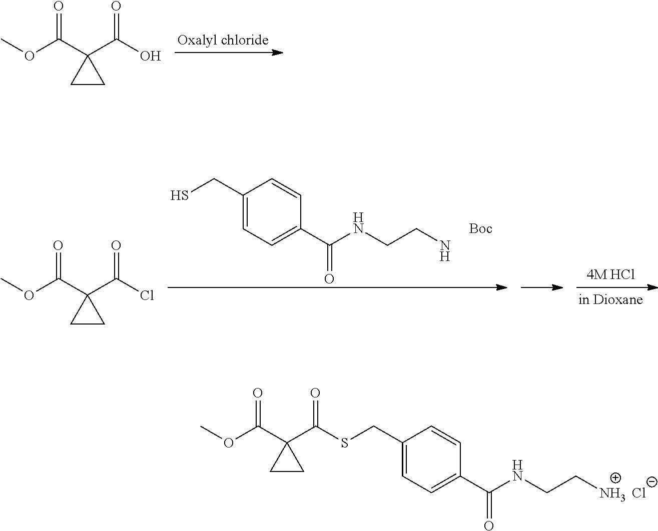

Methyl 1-(((4-((2-aminoethyl)carbamoyl) benzyl)thio) carbonyl) cyclopropane-1-carboxylate (2) To a stirred solution of 1-(methoxycarbonyl)cyclopropane-1-carboxylic acid (200 mg, 1.39 mmol) in CH₂Cl₂ (5 mL) at 0° C. in an inert atmosphere, oxalyl chloride (1.2 mL, 13.9 mmol) was added dropwise and continued stirring for 5 h at room temperature. Removed the solvent and oxalyl chloride under reduced vacuum and redissolved the resulting oily residue in CH₂Cl₂. Added triethylamine (968.0 μl, 6.94 mmol) followed by Boc-ABT (344.5 mg, 1.11 mmol) in CH₂Cl₂ at 0° C. Allowed the reaction mixture to continue stirring under an inert atmosphere at room temperature for 5 h. Progress of the reaction was monitored using thin-layer chromatography (TLC). Purification of the crude material by flash column chromatography (petroleum ether/ethyl acetate, 95:5 to 85:15) furnished white solid product (92.9%, 450 mg). The deprotection was achieved upon treatment with 4 M solution of HCl in 1,4-dioxane for 30 min at room temperature, and the resulting product was used without further purification. : ¹H NMR (500 MHz, DMSO-d₆) δ 8.79 (t, J=5.16 Hz 1H), 8.15 (br s, 3H), 7.88 (d, J=8.16 Hz, 2H), 7.39 (d, J=8.18 Hz, 2H), 4.18 (s, 2H), 3.66 (s, 3H), 3.53 (q, J=5.85 Hz, 2H), 2.99 (m, 3H), 1.54 (m, 4H), ¹³C NMR (126 MHz, DMSO-d₆) δ 194.6 (1C, O═C—S), 170.0 (1C, O═C—O), 166.8 (1C, O═C—NH), 141.4 (1C, Ph), 133.3 (1C, Ph), 129.1 (2C, Ph), 128.1 (2C, Ph), 53.0 (1C, OCH₃), 39.0 (1C, NH₃⁺—CH₂), 37.6 (1C, NH-CH₂), 35.7 (1C, C), 33.0 (1C, SCH₂), 19.1 (2C, CH₂—CH₂). HRMS (ESI): Exact mass cal. for C₁₆H₂₁N₂O₄S⁺[M+H]⁺=337.1217; Found 337.1247.

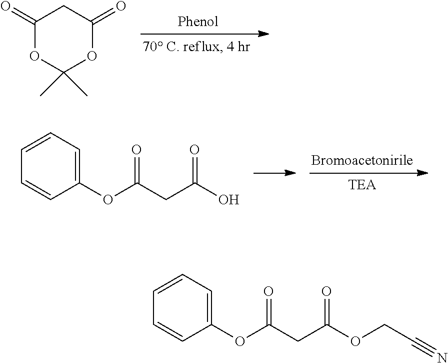

Cyanomethyl phenyl malonate (3): 2,2-Dimethyl-1,3-dioxane-4,6-dione (721 mg, 5 mmol) and phenol (615.7 μl, 7 mmol) were dissolved in toluene (10 mL). The reaction proceeded under reflux for 4 h. The resulting mixture was then added to saturated NaHCO₃. The layers were separated, and the aqueous layer was washed with toluene. The water layer was acidified with 1 M HCl, and the organic layer was separated using ethyl acetate, dried over MgSO₄, filtered, and concentrated under vacuum. Prepared according to the general procedure C using the resulting product, bromoacetonitrile (5 mL, 718 mol, excess) and triethylamine (139 μl, 1 mmol). The reaction mixture was stirred at room temperature for 16 h. The resulting solution was washed with 1 M HCl, 4% NaHCO₃, and brine. The organic layer was dried over MgSO₄, filtered, and concentrated under vacuum. The product was obtained as a colorless liquid (217 mg, 19.8%). : ¹H NMR (500 MHz, DMSO-d) δ 7.46 (m, 2H), 7.30 (m, 1H), 7.19 (m, 2H), 5.12 (s, 2H), 3.99 (s, 2H), ¹³C NMR (126 MHz, DMSO-d₆) δ 167.9 (1C, O—O═C), 167.2 (1C, O═C—O), 152.4 (1C, Ph), 132.0 (2C, Ph), 128.6 (1C, Ph), 123.9 (2C, Ph), 117.6 (1C, C—N), 52.1 (1C, OCH₂), 41.8 (1C, CH₂, Overlapping Solvent peak). HRMS (ESI) Exact mass cal. for C₁₁H₉NO₄Na⁺[M+Na]⁺=242.0424; Found 242.0428.

### Synthesis of succinate diester

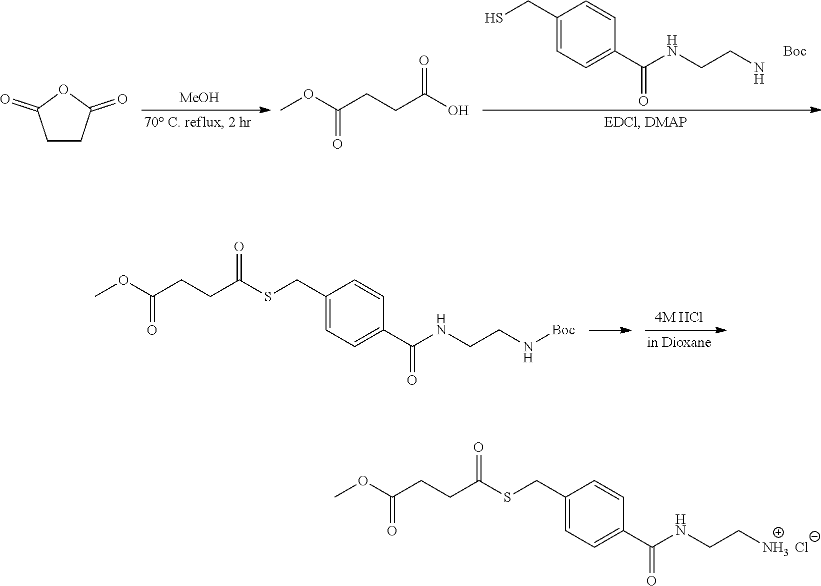

Methyl 4-((4-((2-aminoethyl)carbamoyl)benzyl)thio)-4-oxobutanoate (s): Prepared according to the general procedure A using monomethyl succinate (341 μl, 3 mmol), Boc-ABT (155.2 mg, 0.5 mmol), DMAP (61.1 mg, 0.5 mmol), EDC-HCl (575.1 mg, 3 mmol) and CH₂Cl₂. (10 mL). The deprotection was achieved upon treatment with 4M solution of HCl in 1,4-dioxane for 30 min at room temperature and the product was obtained as a white solid. (102.4 mg, 56.8%): ¹H NMR (500 MHz, DMSO-d₆) δ 8.78 (t, J=5.45 Hz, 1H), 8.14 (br s, 3H), 7.85 (d, J=8.26 Hz, 2H), 7.38 (d, J=8.25 Hz, 2H), 4.18 (s, 2H), 3.59 (s, 3H), 3.53 (q, J=5.92 Hz, 2H), 2.98 (m, 2H), 2.90 (t, J=6.53 Hz, 2H), 2.62 (t, J=6.53 Hz, 2H), ¹³C NMR (126 MHz, DMSO-d₆) δ 197.4 (1C, O═C—S), 172.4 (1C, O═C—O), 166.8 (1C, O═C—NH), 141.8 (1C, Ph), 133.3 (1C, Ph), 129.0 (2C, Ph), 129.1 (2C Ph), 52.0 (1C, CH₃O), 39.03 (1C, CH₂NH₃⁺), 38.3 (1C, CH₂NH), 37.6 (1C, CH₂), 32.3 (1C, SCH₂), 28.9 (1C, CH₂). HRMS (ESI): Exact mass cal. for C₁₅H₂₁N₂O₄S⁺[M+H]⁺=325.1217, Found 325.1242.

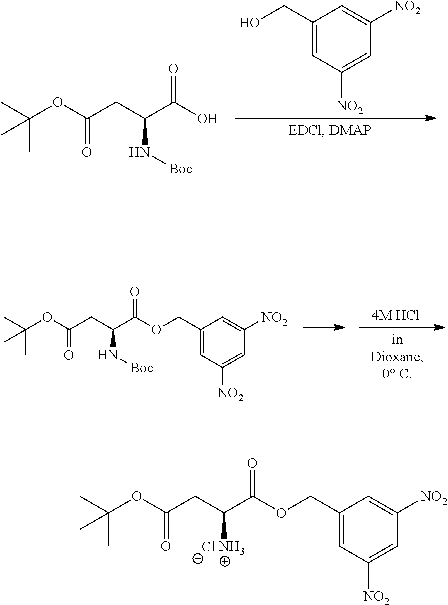 [SMILES: CC(C)(C)OC(=O)C[C@H](NC(=O)OC(C)(C)C)C(=O)O ; O=[N+]([O-])c1cc(CO)cc([N+](=O)[O-])c1 ; CC(C)(C)OC(=O)C[C@H](NC(=O)OC(C)(C)C)C(=O)OCc1cc([N+](=O)[O-])cc([N+](=O)[O-])c1 ; CC(C)(C)OC(=O)C[C@H]([NH3+])C(=O)OCc1cc([N+](=O)[O-])cc([N+](=O)[O-])c1 ; [Cl-]]

4-(tert-butyl) 1-(3,5-dinitrobenzyl) L-aspartate (4) Prepared according to the general procedure B using N-Boc-L-aspartic acid 4-tert-butyl ester (289.3 mg, 1.0 mmol), 3,4-dinitrobenzyl alcohol (237.8 mg, 1.2 mmol), DMAP (122.2 mg, 1.0 mmol), EDC-HCl (230.0 mg, 1.2 mmol) and EtOAc (10 mL). Boc-protected product was successfully obtained. The deprotection was achieved upon treatment with 4M solution of HCl in 1,4-dioxane at 0° C. for 30 min, and the resulting product was washed with diethyl ether to remove 3,4-dinitrobenzyl alcohol. The solvent was concentrated under vacuum, and the product was obtained as a yellow solid. (101.3 mg, 20.5%) ¹H NMR (500 MHz, DMSO-d₆) δ 8.83 (t, J=2.12 Hz, 1H), 8.82 (br s, 3H, overlapped other peaks), 8.76 (d, J=2.11,2H), 5.51 (q, J=4.57 Hz, 2H), 4.42 (t, J=5.30 Hz, 1H), 3.00 (m, 2H), 1.36 (s, 9H)¹³C NMR (126 MHz, DMSO-d) δ 168.9 (1C, O═C), 168.7 (1C, O═C), 148.5 (2C, O═C—NH), 140.0 (1C, Ph), 129.1 (2C, Ph), 118.9 (1C, Ph), 82.1 (1C, OC), 66.8 (1C, OCH₂), 48.9 (1C, CHNH₃⁺), 35.5 (1C, CH₂), 28.0 (3C, CH₃). HRMS (ESI): Exact mass cal. for C₁₅H₂₀N₃O₈⁺[M+H]⁺=370.1245, Found 370.1245.

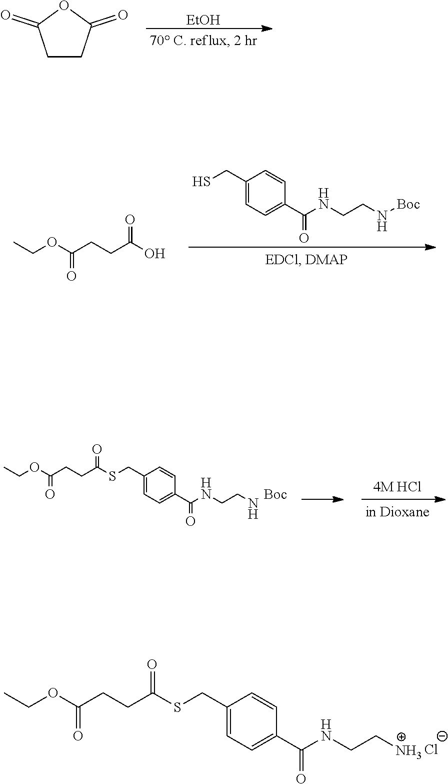

Ethyl 4-((4-((2-aminoethyl)carbamoyl)benzyl)thio)-4-oxobutanoate (5): Succinic anhydride (1.00 g, 10.0 mmol) was dissolved in ethanol (50 mL). The reaction proceeded under reflux for 3 h. The resulting mixture was evaporated to remove ethanol. The monoethyl succinate was obtained. Then, general procedure A using monomethyl succinate (146.1 mg, 1.0 mmol), Boc-ABT (155.2 mg, 0.5 mmol), DMAP (122 mg, 1.0 mmol), EDC-HCl (191.7 mg, 1.0 mmol) and EtOAc (10 mL) was carried out. The deprotection was achieved upon treatment with 4 M solution of HCl in 1,4-dioxane at room temperature for 30 min, and the product was obtained as a white solid (163.1 mg, 87.0%). : ¹H NMR (500 MHz, DMSO-d₆) δ8.78 (t, J=5.29 Hz, 1H), 8.11 (br s, 3H), 7.85 (d, J=8.18 Hz, 2H), 7.37 (d, J=8.15 Hz, 2H), 4.17 (br s, 2H), 4.03 (q, J=7.12, 2H), 3.54-3.52 (m, 2H, Overlapping Solvent peak), 3.01-2.95 (br t, 2H), 2.88 (t, J=6.43 Hz, 2H), 2.63-2.57 (br t, 2H), 1.15 (t, J=7.10 Hz, 3H), 13C NMR (126 MHz, DMSO-d₆) δ197.5 (1C, O═C—S), 172.0 (1C, O═C—O), 166.9 (1C, O═C—NH), 141.8 (1C, Ph), 133.2 (1C, Ph), 129.0 (2C, Ph), 128.1 (2C Ph), 60.6 (1C, CH₂O), 39.02 (1C, CH₂NH₃⁺), 38.3 (1C, CH₂NH), 37.6 (1C, CH₂), 32.2 (1C, SCH₂), 29.1 (1C, CH₂), 14.5 (1C, CH₃). HRMS (ESI): Exact mass cal. for C₁₆H₂₃N₂O₄S⁺[M+H]⁺=339.1373; Found 339.1393.

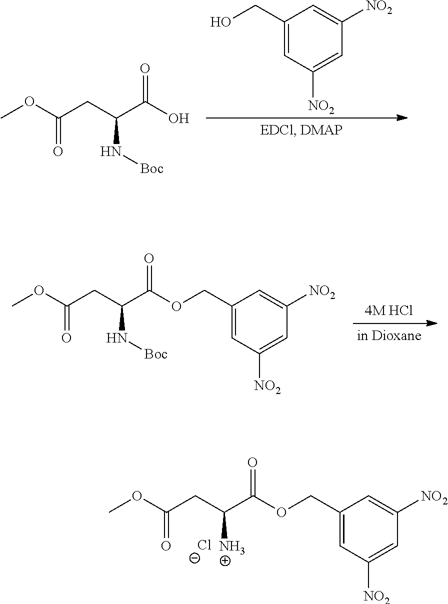 [SMILES: COC(=O)C[C@H](NC(=O)OC(C)(C)C)C(=O)O ; O=[N+]([O-])c1cc(CO)cc([N+](=O)[O-])c1 ; COC(=O)C[C@H](NC(=O)OC(C)(C)C)C(=O)OCc1cc([N+](=O)[O-])cc([N+](=O)[O-])c1 ; COC(=O)C[C@H]([NH3+])C(=O)OCc1cc([N+](=O)[O-])cc([N+](=O)[O-])c1 ; [Cl-]]

1-(3,5-dinitrobenzyl) 4-methyl L-aspartate (6) Prepared according to the general procedure B using (S)-2-((tert-butoxycarbonyl) amino)-4-methoxy-4-oxobutanoic acid (247.2 mg, 1 mmol), 3,4-dinitrobenzyl alcohol (198.1 mg, 1.2 mmol), DMAP (122.1 mg, 1 mmol), EDC-HCl (237.8 mg, 1.2 mmol) and CH₂Cl₂ (10 mL). Boc-protected product was successfully obtained. The deprotection was achieved upon treatment with 4 M solution of HCl in 1,4-dioxane at 0° C. for 30 min, and the resulting product was washed with diethyl ether to remove 3,4-dinitrobenzyl alcohol.

The solvent was concentrated under vacuum, and the product was obtained as a yellow solid. (73.7 mg, 20.3%) ¹H NMR (500 MHz, DMSO-d₆) δ 8.90-8.51 (br, 3H), 8.82 (t, J=1.90 Hz, 1H), 8.74 (d, J=1.90 Hz, 2H), 5.50 (q, J=11.73 Hz, 2H), 4.49 (t, J=5.43 Hz, 1H), 3.62 (s, 3H), 3.12 (m, J=7.59 Hz, 2H)¹³C NMR (126 Hz, DMSO-d₆) δ 170.1 (1C, O═C), 168.5 (1C, O═C), 148.5 (2C, C—N(═O) ═O)), 140.0 (1C, Ph), 128.9 (2C, Ph), 118.8 (1C, Ph), 65.6 (1C, 5° C. H₂), 52.6 (1C, CH₃O), 48.9 (1C, CHNH₃⁺), 34.4 (1C, CH₂). HRMS (ESI): Exact mass cal. for C₁₂H₁₄N₃Oₛ[M+H]⁺=328.0775; Found 328.0815.

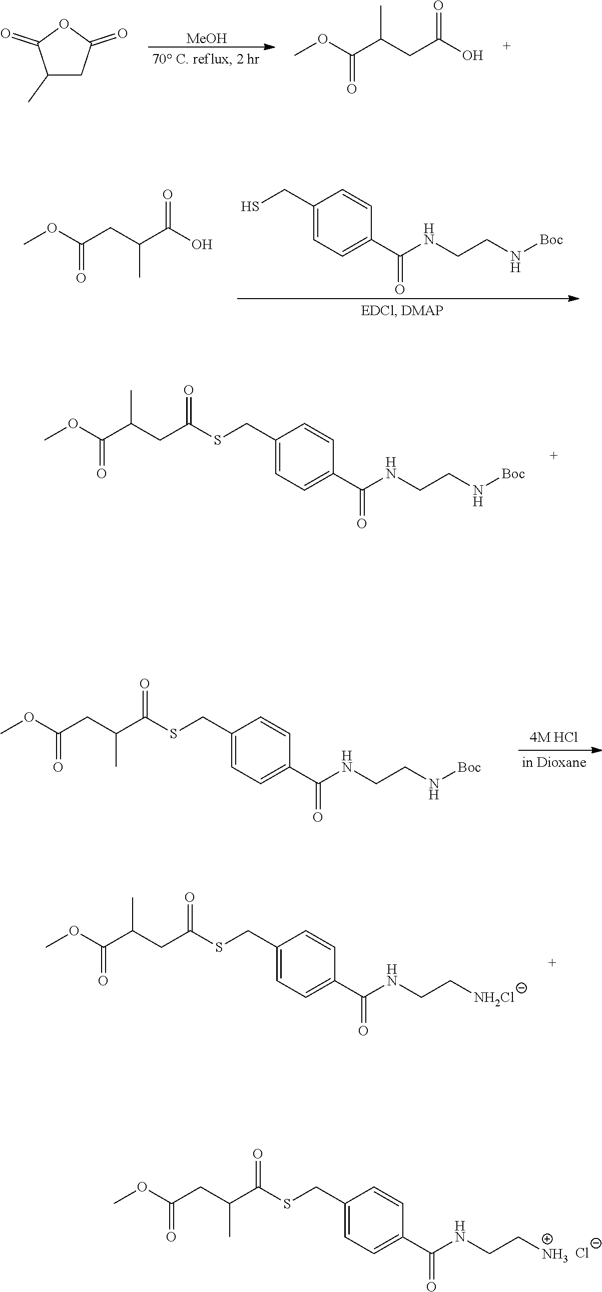

Methyl 4-((4-((2-amninoethyl)carbamoyl)benzyl)thio)-2-methyl-4-oxobutanoate+Methyl 4(4(2 aininoethyl)carbamoyl) benzyl) thia)-3-methyl-4-oxobutanoate (7): Methyl succinic anhydride (280.6 μL, 3 mnmol) was dissolved in methanol (5 mL). The reaction proceeded under reflux for 3 h. The resulting mixture was evaporated to remove methanol, and the methoxy methyloxobutanoic acid was obtained. Then, general procedure A using methoxy methyloxobutanoic acid (438.4 mg, 3.0 mmol), Boc-ABT (341.4 mg, 1.1 mmol), DMAP (61.1 mg, 0.5 mmol), EDC-HCl (230 mg, 1.2 mmol) and CH₂Cl₂. (10 mL) was carried out. The deprotection was achieved upon treatment with 4 M solution of HCl in 1,4-dioxane at room temperature for 30 min, and the product was obtained as a white solid (406.9 mg, 98.7%). As a result of the NMR analysis, two products were made. (Methyl 4-((4-((2-aminoethyl)carbamoyl)benzyl)thio)-2-methyl-4-oxobutanoate) (50%): ¹H NMR (500 MHz, DMSO-d₆) δ 8.82 (t, J=5.25 Hz, 1H), 8.19 (br s, 3H), 7.89 (d, J=8.08 Hz, 2H), 7.39 (d, J=7.85 Hz, 2H), 4.19 (s, 2H), 3.59 (s, 3H), 3.57-3.51 (m, 2H), 3.12-2.55 (m, 5H), 1.17 (d, J=7.07, 3H). ¹³C NMR (126 Hz, DMSO-d₆) δ 197.0 (1C, O═C—S), 175.1 (1C, O═C—O), 166.8 (1C, O═C—NH), 141.8 (1C, Ph), 133.3 (1C, Ph), 129.1 (2C, Ph), 129.0 (2C, Ph), 52.2 (1C, CH₃O), 46.5 (1C, CH₂), 39.0 (1C, CH₂NH₃), 37.6 (1C, CH₂NH) 35.9 (1C, CH), 32.1 (1C, S—CH₂), 16.9 (1C, CH₃) Methyl 4-((4-((2-aminoethyl)carbamoyl)benzyl)thio)-3-methyl-4-oxobutanoate (50%): ¹H NMR (500 MHz, DMSO-d₆) δ 8.82 (t, J=5.25 Hz, 1H), 8.19 (br s, 3H), 7.89 (d, J=8.08 Hz, 2H), 7.39 (d, J=7.85 Hz, 2H), 4.19 (s, 2H), 3.60 (s, 3H), 3.57-3.51 (m, 2H), 3.12-2.55 (m, 5H), 1.12 (d, J=6.12, 3H). ¹³C NMR (126 Hz, DMSO-d₆) δ 201.5 (1C, O═C—S), 171.8 (1C, O═C—O), 166.8 (1C, O═C—NH), 141.8 (1C, Ph), 133.3 (1C, Ph), 129.1 (2C, Ph), 129.0 (2C, Ph), 52.0 (1C, CH₃O), 44.2 (1C, CH), 39.0 (1C, CH₂NH₃⁺), 37.6 (1C, CH₂NH) 37.3 (1C, CH₂), 32.3 (1C, S—CH₂), 18.1 (1C, CH₃). HRMS (ESI): Exact mass cal. for C₁₆H₂₃N₂O₄S⁺[M+H]⁺=339.1373; Found 339.1392.

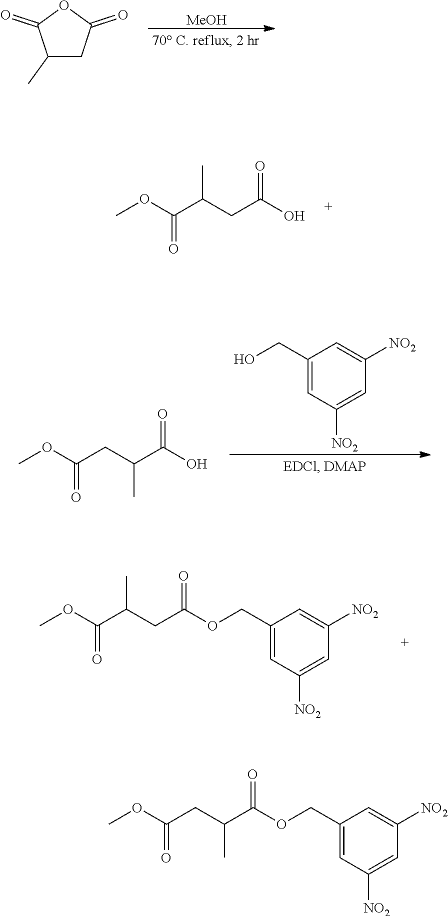

1-(3,5-dinitrobenzyl) 4-methyl 3-methylsuccinate+1-(3,5-dinitrobenzyl) 4-methyl 2-methylsuccinate (7-2): Methyl succinic anhydride (280.6 μl, 3 mmol) and NaOH (40.0 mg, 1 mmol) was dissolved in methanol (5 mL). The reaction proceeded under reflux for 3 h. The resulting solution was washed with 1 M HCl and brine. Prepared according to the general procedure B using resulting product, 3,4-dinitrobenzyl alcohol (594.4 mg, 3 mmol), DMAP (122.2 mg, 1 mmol), EDC-HCl (575.1 mg, 3 mmol) and CH₂Cl₂ (10 ml). Purification by silica gel flash chromatography (30% EtOAc in n-Hex) yielded a yellow solid. (314.8 mg, 32.2%) As a result of the NMR analysis, two products were made. : 1-(3,5-dinitrobenzyl) 4-methyl 2-methylsuccinate (50%) ¹H NMR (500 MHz, DMSO-d₆) δ 8.81-8.78 (m, 1H), 8.64 (t, J=2.42 Hz, 2H), 5.36 (s, 2H), 3.57 (s, 3H), 3.01-2.80 (m, 1H), 2.74-2.58 (m, 2H), 1.21-1.12 (m, 3H), 13C NMR (126 MHz, DMSO-d₆) δ 174.2 (1C, O═C), 173.5 (1C, O═C), 150.4 (2C, C—N(═O)═O), 143.0 (1C, Ph), 130.4 (1C, Ph), 130.2 (1C, Ph), 120.5 (1C, Ph), 66.0 (1C, OCH₂), 54.0 (1C, CH₃O), 39.1 (1C, CH₂), 37.5 (1C, CH), 19.0 (1C, CH₃), 1-(3,5-dinitrobenzyl) 4-methyl 3-methylsuccinate (50%): ¹H NMR (500 MHz, DMSO-d₆) δ 8.81-8.78 (m, 1H), 8.64 (t, J=2.42 Hz, 2H), 5.37 (s, 2H), 3.58 (s, 3H), 3.01-2.80 (m, 1H), 2.98-2.59 (m, 2H), 1.21-1.12 (m, 3H), 13C NMR (126 MHz, DMSO-d₆) δ 174.2 (1C, O═C), 173.5 (1C, O═C), 150.4 (2C, 2C, C—N(═O)═O), 142.9 (1C, Ph), 130.4 (1C, Ph), 130.2 (1C, Ph), 120.4 (1C, Ph), 66.0 (1C, OCH₂), 53.8 (1C, CH₃O), 38.9 (1C, CH₂), 37.5 (1C, CH), 18.9 (1C, CH₃). HRMS (ESI): Exact mass cal. for C₁₃H₁₄N₂O₈Na⁺[M+Na]⁺=349.0642; Found 349.0641.

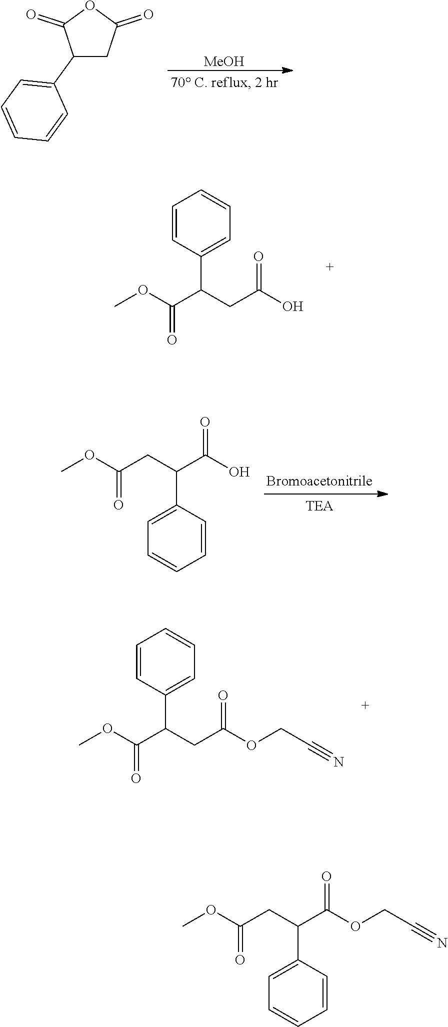 [SMILES: "MeOH" ; O=C1CC(c2ccccc2)C(=O)O1 ; COC(=O)C(CC(=O)O)c1ccccc1 ; COC(=O)CC(C(=O)O)c1ccccc1 ; COC(=O)C(CC(=O)OCC#N)c1ccccc1 ; COC(=O)CC(C(=O)OCC#N)c1ccccc1]

1-(cyanomethyl) 4-methyl 3-phenylsuccinate+1-(cyanomethyl) 4-methyl 2-phenylsuccinate (8): Phenyl succinic anhydride (528.5 mg, 3 mmol) was dissolved in methanol (5 mL). The reaction proceeded under reflux for 5 h. The resulting mixture was evaporated to remove methanol. methoxy phenyloxobutanoic acid was obtained. Prepared according to the general procedure C using the resulting product, bromoacetonitrile (348.3 μl, 5 mmol) and triethylamine (139.5 μl, 1 mmol). The reaction mixture was stirred at room temperature for 16 h. The resulting solution was washed with 1 M HCl, 4% NaHCO₃, and brine. The organic layer was dried over MgSO₄, filtered, and concentrated under vacuum. The product was obtained as a colorless liquid (32.8 mg, 4.42%). As a result of the NMR analysis, two products were made: 1-(cyanomethyl) 4-methyl 3-phenylsuccinate (33%): ¹H NMR (500 MHz, DMSO-d₆) δ 7.39-7.25 (m, 5H), 4.95 (s, 2H), 4.11-4.06 (i, 1H) 3.57 (s, 3H, Overlapping Solvent peak), 3.22-2.67 (n, 2H), ¹³C NMR (126 MHz, DMSO-d₆) δ 173.4 (1C, O═C), 173.2 (1C, O═C), 137.7 (1C, Ph), 129.3 (2C, Ph), 128.3 (2C, Ph) 128.0 (1C, Ph), 116.2 (1C, C═N), 52.6 (1C, OCH₃), 49.5 (1C, OCH₂), 46.7 (1C, CH), 36.9 (1C, CH₂), 1-(cyanomethyl) 4-methyl 2-phenylsuccinate (67%): ¹H NMR (500 MHz, DMSO-d₆) δ 7.39-7.25 (i, 5H), 4.97 (s, 2H), 4.19-4.13 (i, 1H), 3.59 (s, 3H, Overlapping Solvent peak), 3.22-2.67 (i, 2H), ¹³C NMR (126 MHz, DMSO-d₆) δ 172.1 (1C, O═C), 171.8 (1C, O═C), 137.1 (1C, Ph), 129.4 (2C, Ph), 128.2 (2C, Ph), 128. 1 (1C, Ph), 116.0 (1C, C═N), 52.2 (1C, OCH₃), 49.8 (1C, OCH₂), 46.4 (1C, CH), 37.3 (1C, CH₂). HRMS (ESI): Exact mass cal. for C₁₃H₁₃NO₄Na⁺[M+Na]⁺=270.0737; Found 270.0750.

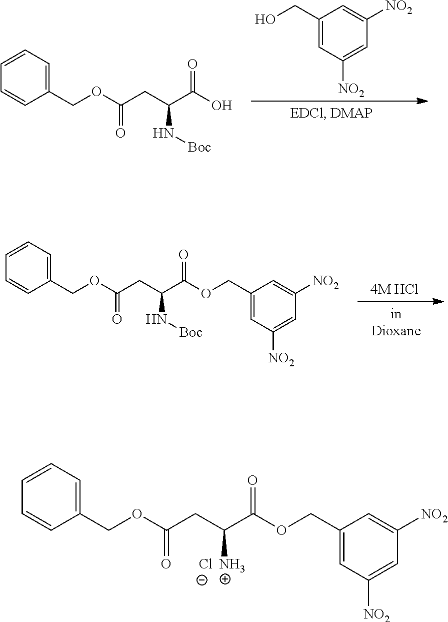 [SMILES: CC(C)(C)OC(=O)N[C@@H](CC(=O)OCc1ccccc1)C(=O)O ; O=[N+]([O-])c1cc(CO)cc([N+](=O)[O-])c1 ; CC(C)(C)OC(=O)N[C@@H](CC(=O)OCc1ccccc1)C(=O)OCc1cc([N+](=O)[O-])cc([N+](=O)[O-])c1 ; [NH3+][C@@H](CC(=O)OCc1ccccc1)C(=O)OCc1cc([N+](=O)[O-])cc([N+](=O)[O-])c1 ; [Cl-]]

4-benzyl 1-(3,5-dinitrobenzyl) L-aspartate (9) Prepared according to the general procedure B using N-Boc-L-aspartic 4-benzyl ester (323.3 mg, 1.0 mmol), 3,4-dinitrobenzyl alcohol (237.8 mg, 1.2 mmol), DMAP (122.2 mg, 1.0 mmol), EDC-HCl (230.0 mg, 1.2 mmol) and EtOAc (10 mL). Boc-protected product was successfully obtained. The deprotection was achieved upon treatment with 4 M solution of HCl in 1,4-dioxanev at 0° C. for 30 min, and the resulting product was washed with diethyl ether to remove 3,4-dinitrobenzyl alcohol. The solvent was concentrated under vacuum. The product was obtained as a yellow solid (182.5 mg, 41.5%). ¹H NMR (500 MHz, DMSO-d₆) δ 8.86 (br, 3H), 8.80 (t, J=2.10 Hz, 1H), 8.71 (d, J=2.10 Hz, 2H), 7.35-7.30 (m, 5H), 5.46 (s, 2H), 5.12 (s, 2H), 4.53 (t, J=5.51 Hz, 1H), 3.23-3.10 (m, J=5.51 Hz, 2H) 13C NMR (126 MHz, DMSO-d₆) δ 169.6 (1C, O═C), 168.5 (1C, O═C), 148.5 (2C, C—N(═O)═O), 140.0 (1C, Ph), 135.9 (1C, Ph), 128.9 (2C, Ph), 128.8 (2C, Ph), 128.6 (1C, Ph), 128.5 (2C, Ph), 118.8 (1C, Ph), 66.8 (1C, OCH₂), 65.6 (1C, OCH₂), 49.0 (1C, CHNH₃⁺), 34.6 (1C, CH₂). HRMS (ESI): Exact mass cal. for C₁₈H₁₈N₃O⁺[M+H]⁺=404.1088; Found 404.1094.

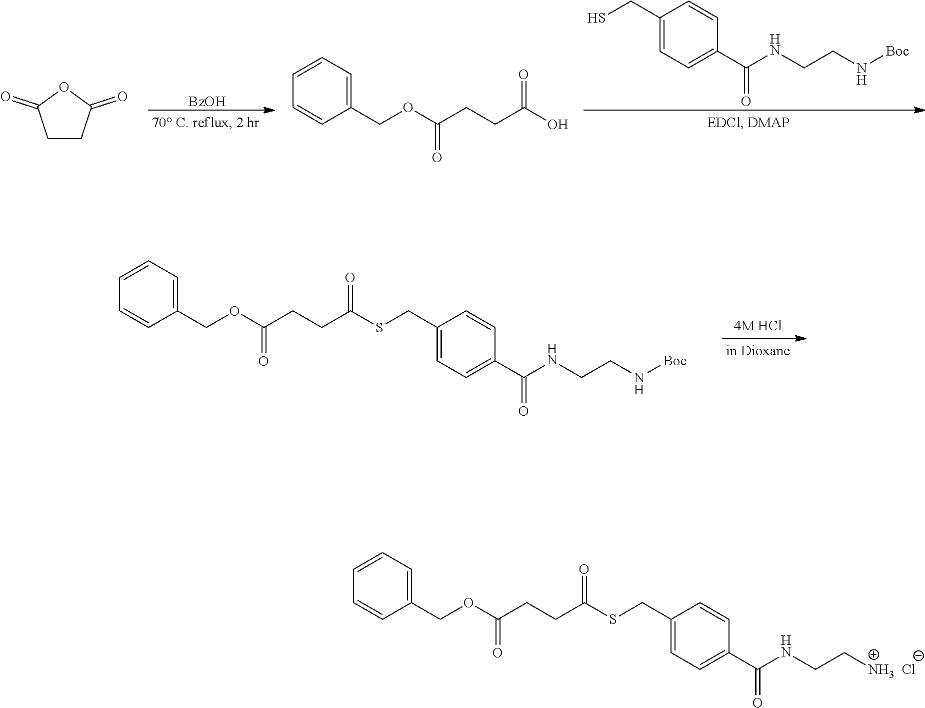

Benzyl 4-((4-((2-aminoethyl)carbamoyl)benzyl)thio) -4-oxobutanoate (10): Succinic anhydride (1.00 g, 10 mmol) was dissolved in benzyl alcohol (50 mL). The reaction proceeded under reflux for 3 h. The resulting mixture was evaporated to remove ethanol. The monoethyl succinate was obtained. Prepared according to the general procedure C using monobenzyl succinate (208.2 mg, 1.0 mmol), Boc-ABT (155.2 mg, 0.5 mmol), DMAP (122.2 mg, 1.0 mmol), EDC-HCl (191.7 mg, 1.0 mmol) and EtOAc. (10 mL). The deprotection was achieved upon treatment with 4 M solution of HCl in 1,4-dioxane at 0° C. for 30 min, and the product was obtained as a white solid (171.8 mg, 78.6%). : ¹H NMR (500 MHz, DMSO-d₆) δ 8.78 (t, J=5.27 Hz, 1H), 8.12 (br, 3H), 7.87 (d, J=8.15 Hz, 2H), 7.41-7.31 (m, 7H), 5.09 (s, 2H), 4.18 (s, 2H), 3.52 (q, J=3.52, 2H), 2.98 (m, 2H), 2.93 (t, J=6.47 Hz, 2H), 2.69 (t, J=6.47 Hz, 2H), 13C NMR (126 Hz, DMSO-d₆) δ 197.4 (1C, O═C—S), 171.9 (1C, O═C—O), 166.8 (1C, O═C—NH), 141.7 (1C, Ph), 136.5 (1C, Ph), 133.3(1C, Ph), 129.0 (2C, Ph), 128.9 (2C Ph), 128.5 (1C, Ph), 128.4 (2C, Ph), 128.1 (2C, Ph), 66.2 (1C, CH₂O), 39.04 (1C, CH₂NH₃⁺), 38.3 (1C, CH₂NH), 37.6 (1C, CH₂), 32.3 (1C, SCH₂), 29.1 (1C, CH₂). HRMS (ESI): Exact mass cal. for C₂₁H₂₅N₂O₄S⁺[M+H]⁺=401.1530; Found 401.1549.

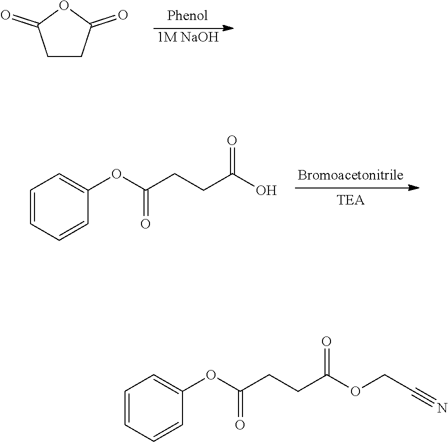

Cyanomethyl phenyl succinate (11) Succinic anhydride (400.3 mg, 4 mmol) and phenol (615.7 μL, 7 mmol) was dissolved in 1 M NaOH (10 mL). The reaction mixture was stirred at room temperature for 6 h. The resulting mixture was treated with cold 1 M HCl. The product solidified, and the neutralizing solution was removed by filtration. Remaining phenol was removed with an additional wash using cold water. Prepared according to the general procedure C using resulting product, bromoacetonitrile (5 mL, 71.8 mmol, excess) and triethylamine (139.5 μL, 1 mmol). The reaction mixture was stirred at room temperature for 16 h. The resulting solution was washed with 1 M HCl, 4% NaHCO₃, and brine. The organic layer was dried over MgSO₄, filtered, and concentrated under vacuum. The product was obtained as a colorless liquid. (182.0 mg, 19.5%): ¹H NMR (500 MHz, DMSO-d₆) δ 7.44-7.38 (m, 2H), 7.30-7.23 (m, 1H), 7.15-7.09 (m, 2H), 7.37 (d, J=8.15 Hz, 2H), 4.78 (s, 2H), 2.99-2.91 (m, 2H), 2.90-2.82 (m, 2H), ¹³C NMR (126 MHz, DMSO-d₆) δ 170.7 (1C, O═C), 170.5 (1C, O═C), 150.5 (1C, Ph), 129.5 (2C, Ph), 126.1 (1C, Ph), 121.4 (2C, Ph), 114.2 (1C, C═N), 48.6 (1C, OCH₂), 28.9 (1C, CH₂), 28.5 (1C, CH₂). HRMS (ESI) Exact mass cal. for C₁₂H₁₁NO₄Na⁺[M+Na]⁺=256.0580; Found 256.0584.

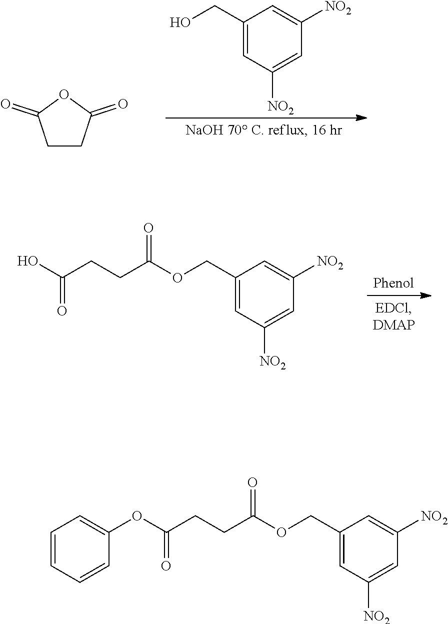 [SMILES: O=C1CCC(=O)O1 ; O=[N+]([O-])c1cc(CO)cc([N+](=O)[O-])c1 ; O=C(O)CCC(=O)OCc1cc([N+](=O)[O-])cc([N+](=O)[O-])c1 ; O=C(CCC(=O)Oc1ccccc1)OCc1cc([N+](=O)[O-])cc([N+](=O)[O-])c1]

3,5-dinitrobenzyl phenyl succinate (11-2) Succinic anhydride (500.4 mg, 5 mmol), 3,4-dinitrobenzyl alcohol (990.7 mg, 5 mmol) NaOH (40.0 mg, 1 mmol) was dissolved in EtOAc (10 mL). The reaction proceeded under reflux for 16 h. The resulting solution was washed with 1 M HCl, and brine. The organic layer was dried over MgSO₄, filtered, and concentrated under vacuum. Purification by silica gel flash chromatography (50% EtOAc in n-Hex) yielded a product. The product was dissolved in EtOAc (10 mL) and treated with phenol (263.9 μL, 3 mmol), EDC-HCl (575.1 mg, 3 mmol) and DMAP (122.2 mg, 1 mmol). The reaction mixture was stirred at room temperature for 16 h. The resulting solution was washed with 1 M HCl, 4% NaHCO₃, and brine. The organic layer was dried over MgSO₄, filtered, and concentrated under vacuum. Purification by silica gel flash chromatography (50% EtOAc in n-Hex) yielded a yellow solid. (26.3 mg, 2.34%). : ¹H NMR (500 MHz, DMSO-d₆) δ 8.78 (t, J=2.10, 1H), 8.67 (d, J=2.10, 2H), 7.41-7.37 (m, 2H), 7.27-7.23 (m, 1H), 7.06-7.02 (m, 2H), 5.40 (s, 2H), 2.91-2.87 (m, 2H), 2.83-2.80 (m, 2H), ¹³C NMR (126 MHz, DMSO-d₆) δ 172.2 (1C, O═C), 171.3 (1C, O═C), 150.8 (1C, O-Ph(C)), 148.5 (2C, C—N(═O)═O), 141.1 (1C, Ph), 129.9 (2C, Ph), 128.7 (2C, Ph), 126.3 (1C, Ph), 122.0 (2C, Ph), 118.6 (1C, Ph), 64.3 (1C, OCH₂), 49.0 (1C, CH), 29.1 (1C, CH₂). HRMS (ESI): Exact mass cal. for C₁₂H₁₄N₂ONa+[M+Na]⁺=397.0642; Found 397.0633.

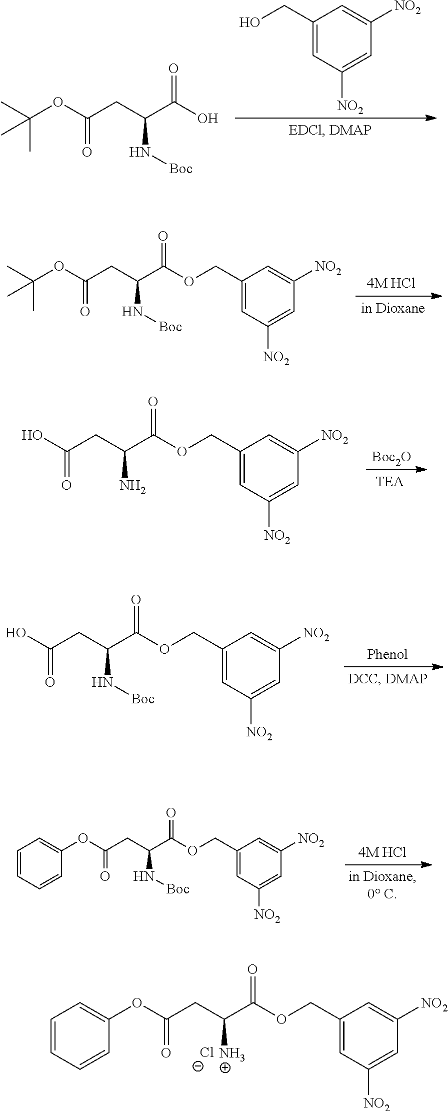

1-(3,5-dinitrobenzyl) 4-phenyl L-aspartate (12): Prepared according to the general procedure B, using N-Boc-L-aspartic acid 4-tert-butyl ester (1.45 g, 5.0 mmol), 3,4-dinitrobenzyl alcohol (1.19 g, 6.0 mmol), DMAP (610.9 mg, 5.0 mmol), EDC-HCl (1.15 g, 6 mmol) and EtOAc (50 mL). Boc and tert-butyl protected product was successfully obtained. The deprotection was achieved upon treatment with 4 M solution of HCl in 1,4-dioxane at room temperature for 1 h, and the resulting product was washed with diethyl ether to remove 3,4-dinitrobenzyl alcohol. The resulting product was dissolved in dioxane (20 mL) and water (20 mL). The solution was added to 1 M NaOH (10 mL). The mixture was cooled to 0° C., Boc₂O (1.09 g, 5 mmol) was slowly added, and the reaction was stirred for 6 h at room temperature. Subsequently, the reaction mixture was diluted with EtOAc (10 mL) and acidified with 1 M HCl. The layers were separated, and the aqueous fraction was extracted with EtOAc. The organic layer was dried over MgSO₄, filtered, and concentrated under vacuum. The resulting product and DCC (1.03 g, 5 mmol) were dissolved in THF (10 mL) and treated with pyridine (402.7 μL, 5.0 mmol). The reaction was stirred for 18 h at room temperature. The resulting solution was filtered to remove precipitants. The resulting mixture was then added to 4% NaHCO₃. The layers were separated, and the aqueous layer was washed with EtOAc. The water layer was acidified with 1 M HCl, and the organic layer was separated using EtOAc, dried over MgSO₄, filtered, and concentrated under vacuum. The deprotection was achieved upon treatment with 4 M solution of HCl in 1,4-dioxane at 0° C. for 30 min, and the product was obtained as a white solid (381.7 mg, 17.9%). ¹H NMR (500 MHz, DMSO-d₆) δ 8.80 (t, J=2.08 Hz, 1H), 8.76 (d, J=1.90 Hz, 2H), 8.59 (br, 3H), 7.41-7.37 (m, J=7.80 Hz, 2H), 7.30-7.21 (m, 1H), 7.11-7.04 (m, 2H), 5.51 (s, 2H), 4.68 (t, J=5.49 Hz, 1H), 3.41-2.85 (m, 2H)¹³C NMR (126 MHz, DMSO-d₆) δ 171.3 (1C, O═C), 168.4 (1C, O═C), 150.4 (1C, Ph), 148.5 (2C, C—N(═O)—O)), 140.0 (1C, Ph), 129.9 (2C, Ph), 129.2 (2C, Ph), 126.6 (1C, Ph), 121.9 (2C, Ph), 118.9 (1C, Ph), 65.4 (1C, OCH₂), 49.0 (1C, CHNH₃⁺), 34.9 (1C, CH₂). HRMS (ESI) Exact mass cal. for C₁₇H₁₆N₃O+[M+H]⁺=390.0932; Found 390.0946.

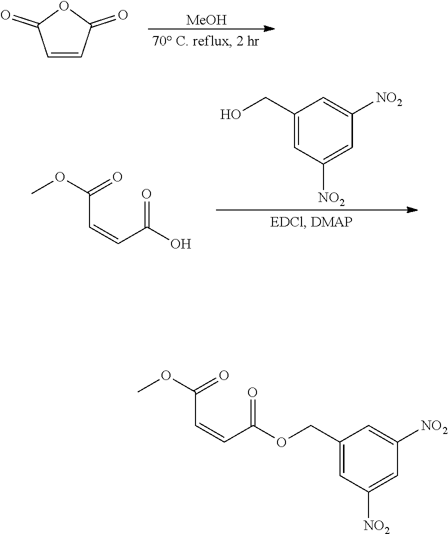

3,5-dinitrobenzyl methyl maleate (13) Maleic anhydride (196.1 mg, 2 mmol) was dissolved in 4 ml of methanol. The reaction proceeded under reflux for 3 h. The resulting solution was washed with 1 M HCl and brine. Then, general procedure B using resulting product, 3,4-dinitrobenzyl alcohol (594.4 mg, 3 mmol), DMAP (122.2 mg, 1 mmol), EDC-HCl (575.1 mg, 3.0 mmol) and CH₂Cl₂ (10 mL) was carried out. Purification by silica gel flash chromatography (50% EtOAc in n-Hex) yielded a yellow solid. (140.4 mg, 22.6%): ¹H NMR (500 MHz, DMSO-d₆) δ 8.81 (t, J=4.14 Hz, 1H), 8.73 (t, J=2.07 Hz, 2H), 6.89 (d, J=6.88 Hz, 2H), 5.49 (s, 2H), 3.76 (s, 3H), ¹³C NMR (126 MHz, DMSO-d₆) δ 165.2 (1C, O═C—O), 164.5 (1C, O═C—O), 148.6 (2C, C—N(═O)═O), 140.4 (1C, Ph), 134.1 (1C, ═CH), 133.2 (1C, ═CH), 128.9 (2C, Ph), 118.7 (1C, Ph), 65.0 (1C, CH₂O), 52.8 (1C, CH₃O). HRMS (ESI): Exact mass cal. for C₁₂H₁₀N₂O₈Na⁺[M+Na]⁺=333.0329; Found 333.0320.

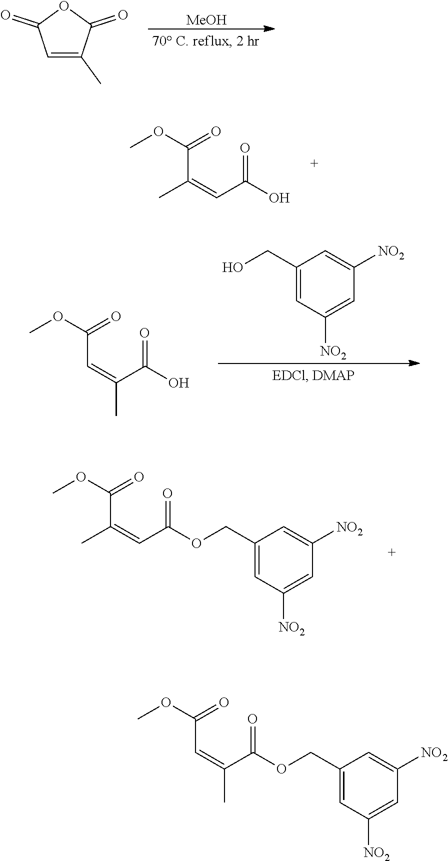

4-(3,5-dinitrobenzyl) 1-methyl 2-methylmaleate+1-(3,5-dinitrobenzyl) 4-methyl 2-methylmaleate (14) Citraconic anhydride (179.8 μl, 2 mmol) and NaOH (20.0 mg, 0.5 mmol) was dissolved in 4 ml of methanol. The reaction proceeded under reflux for 3 h. The resulting solution was washed with 1 M HCl and brine. General procedure B using the resulting product, 3,4-dinitrobenzyl alcohol (594.4 mg, 3 mmol), DMAP (122.2 mg, 1 mmol), EDC·HCl (512.3 mg, 1.2 mmol) and CH₂Cl₂ (10 mL) was carried out. Purification by silica gel flash chromatography (50% EtOAc in n-Hex) yielded a yellow solid. (60.5 mg, 9.32%) As a result of the NMR analysis, two products were made. : 4-(3,5-dinitrobenzyl) 1-methyl 2-methylmaleate (25%) ¹H NMR (500 MHz, DMSO-d₆) δ 8.79 (m, 1H), 8.66 (d, J=3.45 Hz, 2H), 6.22 (s, 1H), 5.41 (s, 2H), 3.66 (s, 3H), 2.04 (s, 3H), ¹³C NMR (126 MHz, DMSO-d₆) δ 168.3 (1C, O═C), 165.5 (1C, O═C), 148.5 (2C, C—N(═O)═O, 145.2 (1C, C═), 140.4 (1C, Ph), 129.7 (2C, Ph), 120.9 (1C, HC═) 118.9 (1C, Ph), 64.4 (1C, OCH₂), 52.2 (1C, CH₃O), 20.3 (1C, CH₃), 1-(3,5-dinitrobenzyl) 4-methyl 2-methylmaleate (75%): ¹H NMR (500 MHz, DMSO-d₆) δ 8.81 (m, 1H), 8.68 (t, J=3.45 Hz, 2H), 6.18 (s, 1H), 5.48 (s, 2H), 3.62 (s, 3H), 2.07 (s, 3H), ¹³C NMR (126 MHz, DMSO-d₆) δ 168.3 (1C, O═C), 165.5 (1C, O═C), 148.5 (2C, C—N(═O)═O), 145.2 (1C, C═),140.4 (1C, Ph), 128.8 (2C, Ph), 121.7 (1C, HC═) 118.7 (1C, Ph), 64.8 (1C, OCH₂), 52.6 (1C, CH₃O), 20.2 (1C, CH₃). HRMS (ESI): Exact mass cal. for C₁₃H₁₂N₂O₈Na⁺[M+Na]⁺=347.0486; Found 347.0485.

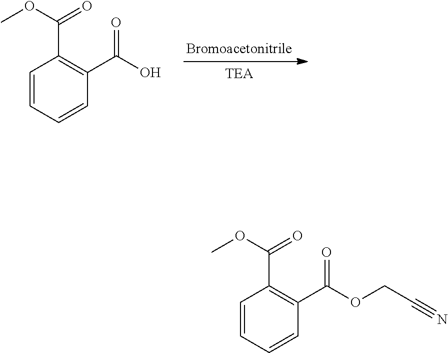 [SMILES: COC(=O)c1ccccc1C(=O)O ; COC(=O)c1ccccc1C(=O)OCC#N]

Cyanomethyl methyl phthalate (15) Prepared according to the general procedure C using monomethyl phthalate (95.5 mg, 0.53 mmol), bromoacetonitrile (1.0 mL, 14.4 mmol) and triethylamine (1.0 mL, 7.17 mmol).

The reaction mixture was stirred at room temperature for 2 h. The resulting solution was washed with 1 M HCl, 4% NaHCO₃, and brine. The organic layer was dried over MgSO₄, filtered, and concentrated under vacuum. The product was obtained as a colorless liquid (109.5 mg, 94.3%). : ¹H NMR (500 MHz, DMSO-d₆) δ 7.73-7.84 (m, 4H), 5.22 (s, 2H), 3.85 (s, 3H), ¹³C NMR (126 MHz, DMSO-d₆) δ 167.3 (1C, O═C), 166.5 (1C, O═C), 132.8 (1C, Ph), 132.5 (1C, Ph), 131.6 (1C, Ph), 130.3 (1C, Ph), 129.6 (1C, Ph), 129.5 (1C, Ph), 116.1 (1C, C--N), 53.3 (1C, OCH₃). 50.6 (1C, OCH₂). HRMS (ESI): Exact mass cal. for Cn₁₁H₉NO₄Na⁺[M+Na]⁺=242.0424; Found 242.0425.

Tables 1 and 2 below show the cyclization efficiency.

TABLE 1

| | | | | Optimal charging | Cyclization |
|---|---|---|---|---|---|
| | substrates | Structure | LG | condition | efficiency |
| malonate | m m-2 | 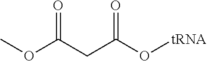 | ABT DNB | 50 mM pH 8.8 aFx Not charged | 100% |
| | 0 | 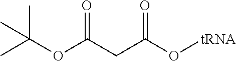 | ABT | Not charged | 100% |
| | 1 | 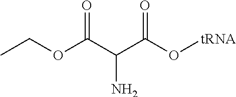 | DNB | 50 mM pH 7.5 dFx | 100% |
| | 2 | 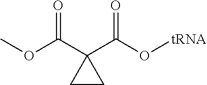 | ABT | 50 mM pH 8.8 aFx | 100% |
| | 3 | 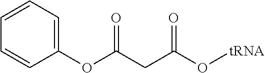 | CME | 25 mM pH 7.5 eFx | 100% |
| Succinate | s | 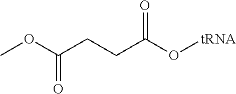 | ABT | 50 mM pH 8.8 aFx | 20% |
| | 4 | 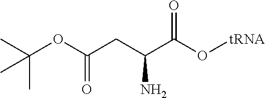 | DNB | 50 mM pH 7.5 dFx | 0% |
| | 5 | 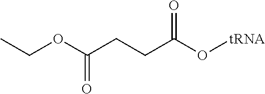 | ABT | 50 mM pH 8.8 aFx | 22% |
| | 6 | 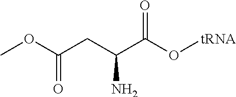 | DNB | 50 mM pH 7.5 dFx | 31% |
| | 7 7-2 | 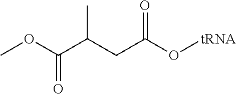 | ABT DNB | 50 mM pH 8.8 aFx Not charged | 33% |
| | 8 | 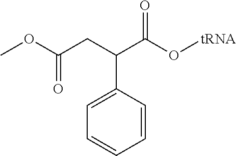 | CME | 50 mM pH 8.8 eFx | 33% |
| | 9 | 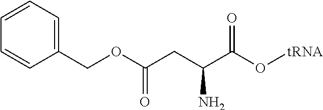 | DNB | 25 mM pH 8.8 dFx | 40% |
| | 10 | 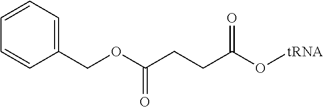 | ABT | 25 mM pH 8.8 aFx | 45% |

TABLE 2

| | | | | |
|---|---|---|---|---|
| 11 11-2 | 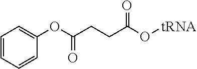 | CME DNB | 50 mM pH 8.8 eFx Not charged | 100% |
| 12 | 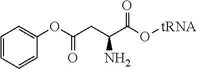 | DNB | 10 mM pH 7.5 dFx | 100% |
| 13 | 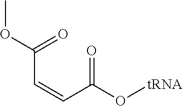 | DNB | Not charged | — |
| 14 | 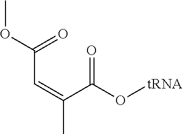 | DNB | Not charged | — |
| 15 | 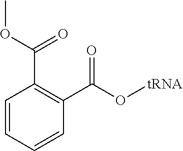 | CME | Not charged | — |

### Synthesis of hydrazino substrates

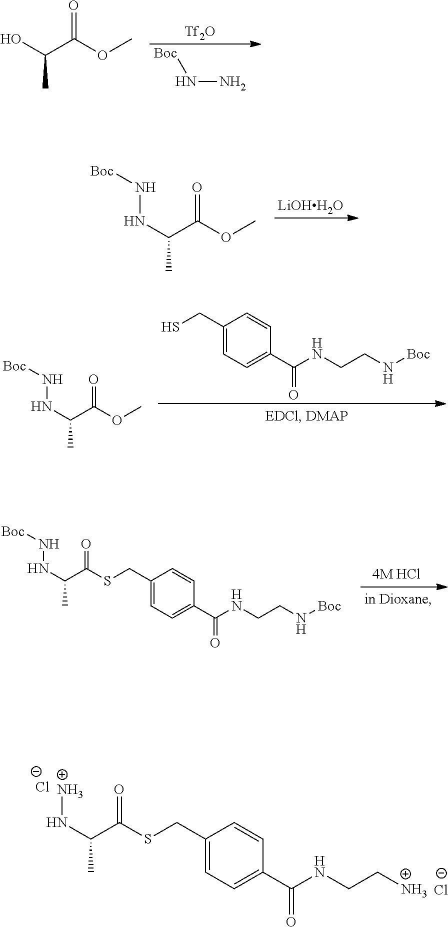

S-(4-((2-aminoethyl)carbamoyl)benzyl) 2-hydrazineylpropanethioate (Hz) (+) -Methyl D-lactate was obtained from a commercial supplier (Sigma-Aldrich) and used as received: To a solution of (+)-methyl D-lactate (1.43 mL, 15.0 mmol) in DCM (45 mL) was added trifluoromethanesulfonic anhydride (3.28 mL, 19.5 mmol) and 2,6-lutidine (3.47 mL, 30.0 mmol) at 0° C., and the reaction was stirred at the same temperature until full consumption of the starting material (confirmed by TLC). To this was then added tert-butyl carbazate (3.96 g, 30.0 mmol), and the resulting mixture was further stirred at 0° C. for 4 h, then at room temperature for 16 h. The reaction mixture was diluted with DCM and washed with H₂O, brine and 1 M HCl. The organic layer was then dried over anhydrous MgSO₄, concentrated under reduced pressure, and purified by flash column chromatography (30% EtOAc/n-Hex) to furnish [(tert-butoxycarbonyl)amino]-L-alanine methyl ester as a yellow oil (2.64 g, 81%).

The methyl ester (2.44 g, 11.2 mmol) obtained above was then dissolved in 1:1 mixture of THF/HO (24 mL) and treated with LiOH-H₂O (940 mg, 22.4 mmol). After stirring at room temperature for 3 h, the mixture was concentrated under reduced pressure and the remaining aqueous layer was washed with EtOAc. The aqueous layer was then acidified to pH ˜1 using 1M HCl (aq, extracted with EtOAc, dried over anhydrous MgSO₄, and concentrated under reduced pressure to give [(tert-butoxycarbonyl)amino]-L-alanine as a thick colorless oil (2.08 g, 91%).

Prepared according to General Procedure C using [(tert-butoxycarbonyl)amino]-L-alanine. (428 mg, 2.1 mmol), Boc-ABT (465 mg, 1.5 mmol), DMAP (512 mg, 4.2 mmol), EDC·HCl (803 mg, 4.2 mmol) and DCM (10 mL). Purification by flash column chromatography (60% EtOAc in n-Hex) afforded the corresponding Boc-protected product as a colorless oil (338 mg, 45%). The deprotection was achieved upon treatment with 4 M solution of HCl in 1,4-dioxane, and the resulting product was used without further purification and characterization. (Boc)₂-(Hz): ¹H NMR (500 MHz, DMSO-d₆) δ 8.40 (t, J=5.11 Hz, 1H), 8.31 (br s, 1H), 7.76 (d, J=7.95 Hz, 2H), 7.37 (d, J=8.15 Hz, 2H), 6.89 (t, J=5.38 Hz, 1H), 4.06 (s, 2H), 3.68 (d, J=5.53 Hz, 1H), 3.28 (q, J=6.04 Hz, 2H), 3.10 (q, J=6.06 Hz, 2H), 1.38 (s, 9H), 1.37 (s, 9H), 1.15 (d, J=6.96 Hz, 3H)¹³C NMR (126 MHz, DMSO-d₆) δ 204.0 (1C, O═C—S), 166.5 (1C, O═C—NH), 156.2 (2C, O═C—O), 141.8 (1C, Ph), 133.6 (1C, Ph), 129.0 (2C, Ph), 127.8 (2C, Ph), 79.3 (1C, O—C), 78.1 (1C, O—C), 65.5 (1C, NH-CH), 42.2 (1C, NH—CH₂—CH₂-NH, Overlapping Solvent peak), 31.8 (1C, S—CH₂), 28.7 (3C, (CH₃)₃), 28.6 (3C, (CH₃)₃), 17.3 (1C, CH₃). HRMS (ESI): Exact mass cal. for C₂₃H₃₇N₄O₆S⁺[M+H]⁺=497.2428; Found 497.2428.

### Example

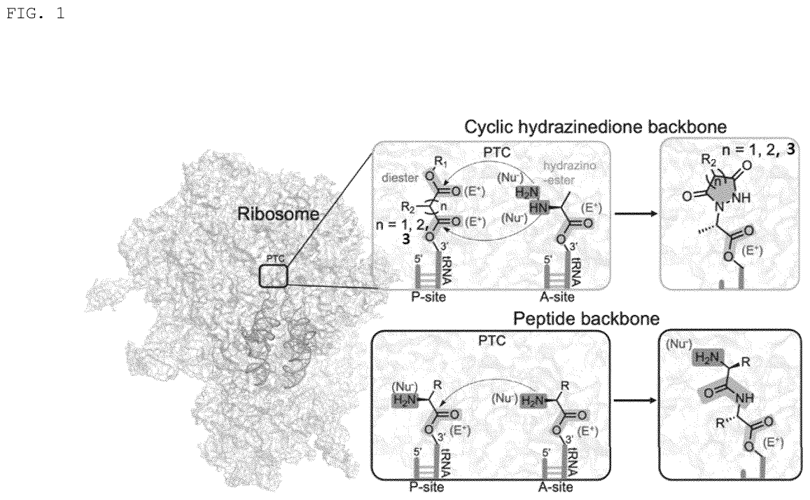

The ribosome, an RNA-based catalyst, facilitates the formation of peptide (amide) bonds through a mechanism known as entropy trapping. By positioning amino-acyl tRNA molecules in close proximity within a confined space, the ribosome reduces their conformational freedom, thereby enhancing the efficiency of peptide bond formation. This approach contrasts with typical catalytic strategies in synthetic chemistry, such as acid-base catalysis, and highlights the ribosome's ability to synthesize biopolymers (e.g., peptides or proteins). Uniquely, the ribosome, working in conjunction with other cellular machinery, can achieve sequence-defined, template-directed polymerization with high fidelity at speeds up to 17 monomers/second. The core reaction, aminolysis, involves the nucleophilic α-amino group of an amino acid linked to the transfer RNA (tRNA) at the ribosome's A-site attacking the electrophilic carbonyl carbon in the ester linkage of the tRNA in the ribosome's P-site (FIG. 1). Despite an evolutionary preference for a set of 20 canonical amino acids, the ribosome can also polymerize a wide array of non-canonical monomers (ncMs) through aminolysis, resulting in the formation of amide backbone. These ncMs vary significantly in size, shape, and chemical characteristics, including a diverse range: α-, β-, γ-, δ-, ε-, ζ-, D-amino acids and non-amino carboxylic acids. Incorporating ncMs into proteins broadens the spectrum of chemical diversity and functionality of biopolymers. This development holds promise for producing novel enzymes and therapeutics characterized by improved or entirely new properties.

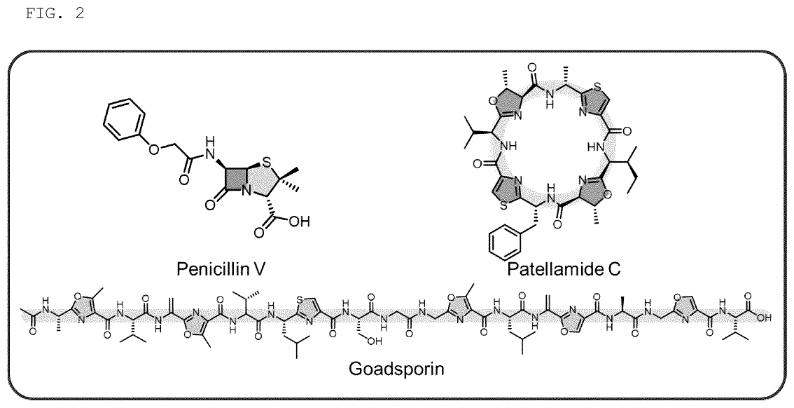

Building on previous efforts to expand the range of substrates, the field has advanced in developing peptide isosteres by leveraging the ribosome's capability for synthesizing linear polypeptides. The replacement of amino acids with structural analogs such as hydroxy-, thio-, and aminocarbothio-acid has demonstrated that the ribosome can directly produce polyesters, polythioesters, and polythioanides, respectively. Another significant advancement that challenges the natural functions of the ribosome is the introduction of cyclic backbones into peptides. These cyclic motifs are important for peptide-based natural products as they enhance proteolytic resistance, structural rigidity, and specificity towards target proteins, often resulting in increased drug efficacy. Such examples include penicillin V (dipeptide of Cys-Val), goadsporin (polyazole antibiotics), and patellamide C (macrocyclic antimicrobial peptides) (FIG. 2). The production of these cyclic structures in peptides was initially sought using indirect methods, where the peptide is first synthesized using a ribosome to contain amino acids with reactive side chains toward enzymatic reactions, followed by an enzymatic ring-closing reaction. Examples of enzymes involved in these reactions include PatD, GodD, and LazDE, cyclohydratases that form heterocyclic azoline derivatives using Cys, Ser, or Thr. More recently, a direct method for cyclic motif production was developed, where ncMs are attached to tRNA using a genetic code reprogramming approach, and ribosomes facilitate a cyclocondensation reaction between the two sequentially incorporated ncMs in vitro.¹⁷ This discovery represents the first example of the ribosome's ability to catalyze ring-closure reactions. Despite the advancement, the full scope of reactions that ribosomes can catalyze remains largely unexplored. Expanding the diversity of cyclic backbones could deepen our understanding of ribosomal polymerization and widen the array of precisely tailored motifs within polypeptides particularly relevant in pharmaceutical applications.

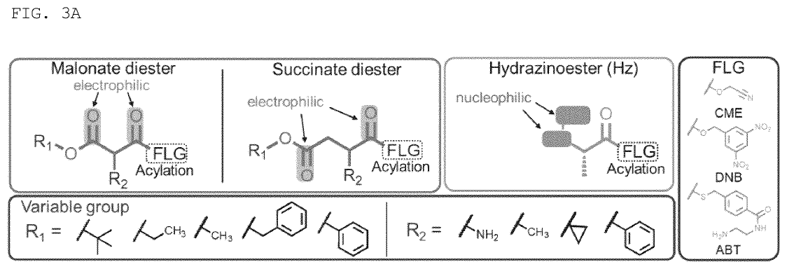

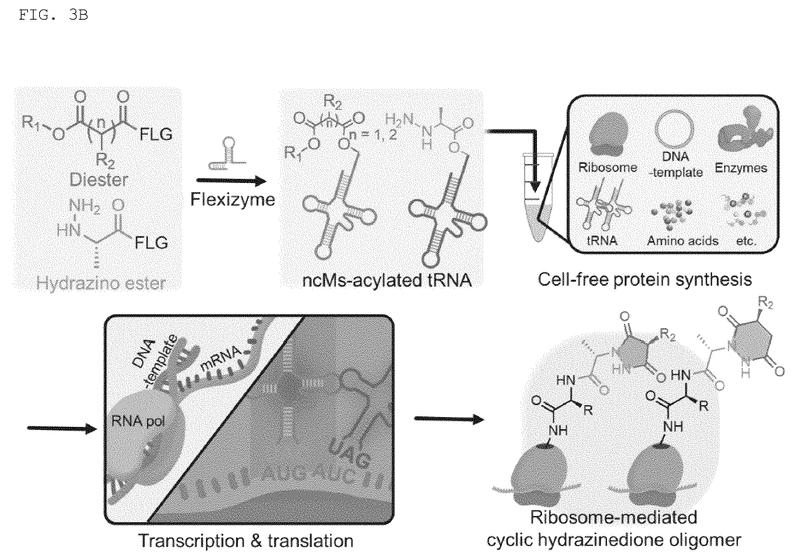

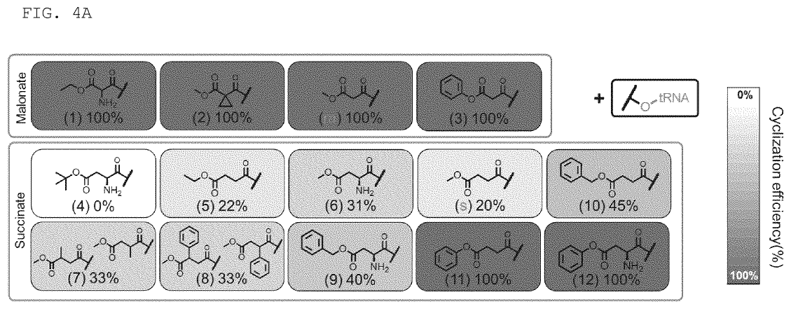

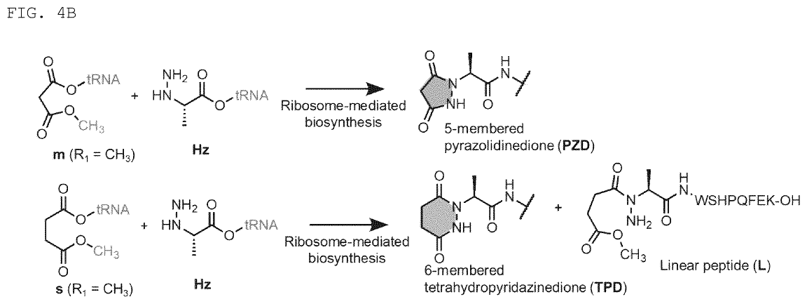

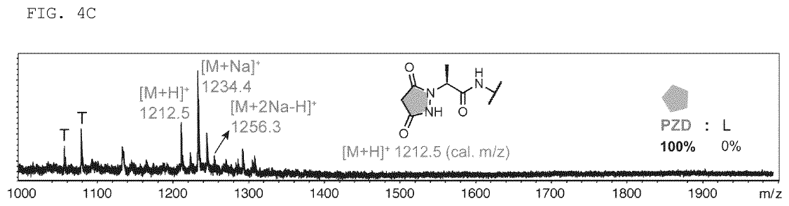

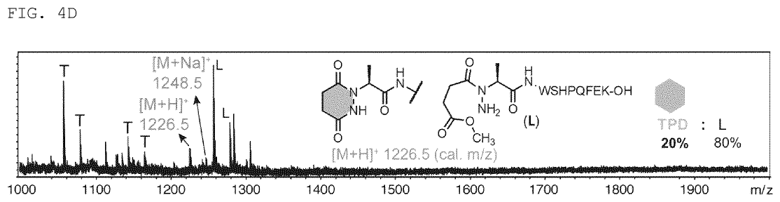

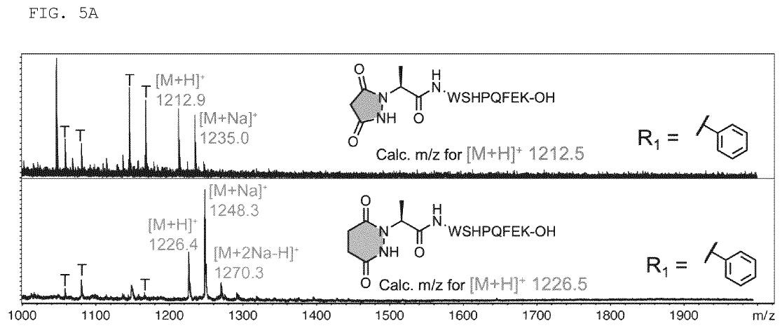

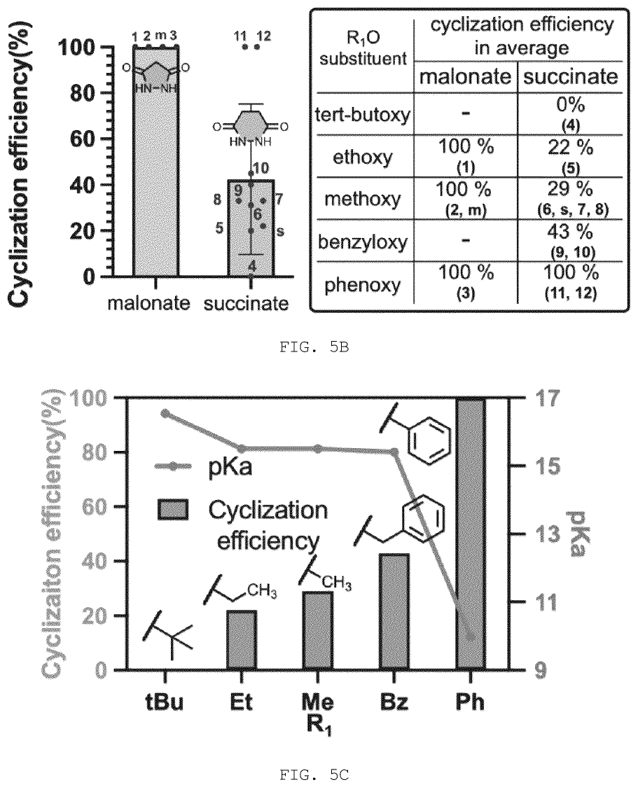

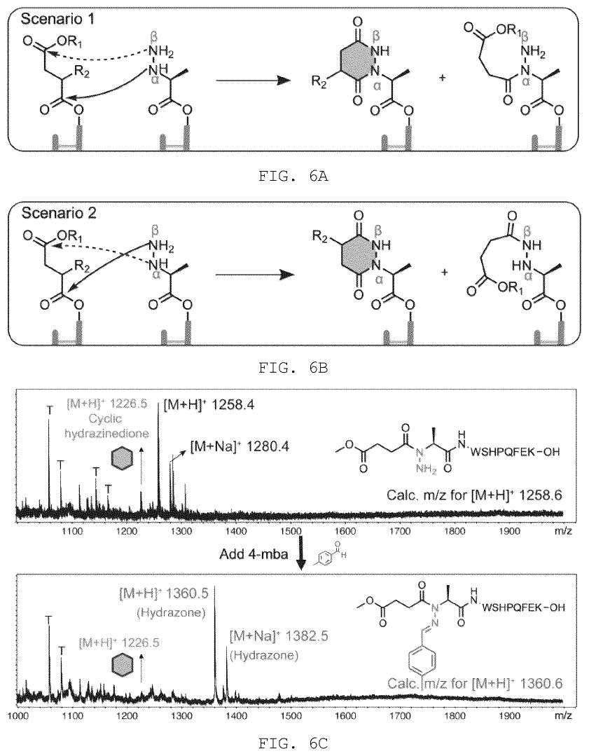

In this invention, the inventors aim to show the ribosome's ability to directly synthesize new and diverse cyclic structures, broadening the spectrum of non-canonical backbones that can be used towards synthesis and discovery of new and improved medicines. To achieve this goal: (i) the inventors design two sets of bifunctional substrates, each containing two electrophiles and nucleophiles within the molecule, respectively (FIG. 1). In the ribosome's catalytic site, these substrates undergo ring-closing reactions to form cyclic backbones rather than a peptide bond (FIG. 1). (ii) By altering the architecture of these substrates, the inventors demonstrate the synthesis of diverse 5—and 6-membered rings (FIG. 3). This invention is the first to demonstrate the ribosome's ability to directly synthesize 5-membered cyclic structures, deepening our understanding of the ribosome's bond-forming mechanism. Additionally, the inventors show modulation of cyclization efficiency by changing the kinetic properties of the substituents on the substrates (FIGS. 4 and 5). (iii) Finally, the inventors present evidence confirming the regioselectivity of the cyclic structures formed within the ribosome, which supports the wild type ribosome's preference for α-amino acids over those with other non-canonical amino acids bearing an extended carbon chain (e.g., R-amino acids) (FIGS. 1 and 6).

### Synthesis of ncMs-acylated tRNAs for ribosome-mediated polymerization in vitro

The synthesis of cyclic backbones using the ribosome requires ncM-acylated tRNAs (FIG. 3). The conventional approach to charging ncMs onto tRNAs involves engineering aminoacyl-tRNA synthetases (aaRSs), the enzymes responsible for attaching natural amino acids to their corresponding tRNAs. However, engineered aaRSs have limited promiscuity and can only accommodate a narrow range of amino acids structurally similar to those used in the engineering process.³⁷ To overcome this limitation, the inventors employed the aminoacylating ribozyme, flexizyme (Fx), which catalyzes tRNA acylation by recognizing an aromatic group³⁸ of an activated ester substrate. Fx has demonstrated extensive compatibility with a diverse range of ncMs with aromatic rings,^{10, 12, 39} thus expanding the scope of ribosome-mediated synthesis of novel bio-based materials.⁴⁰ Currently, four different ester or thioester leaving groups (flexizyme leaving group (FLG), FIG. 3A) are used in conjunction with three flexizyme variants (eFx, dFx, and aFx): eFx/cyanomethyl ester (CME), dFx/dinitrobenzyl ester (DNB) or chlorobenzyl thioester (CBT), and aFx/(2-aminoethyl) amidocarboxybenzyl thioester (ABT). The selection of the appropriate Fx and corresponding FLG depends on the physicochemical characteristics of the substrate.⁴¹ For example, substrates bearing an aromatic group within their structure (e.g., phenylalanine) are optimally suited for CME. In contrast, substrates lacking an aromatic residue (e.g., lysine) require DNB or CBT, while hydrophobic substrates (e.g., leucine) are best matched to ABT due to the water-solubilizing moiety (—NH₃⁺ on the aromatic component.³⁹ The recently reported ′Fx substrate design rules' have more broadened the scope of Fx applications by providing guidelines for identifying ncMs compatible with Fx-mediated acylation.³⁸

### Design of chemical substrates for ribosome-mediated cyclic bond formation

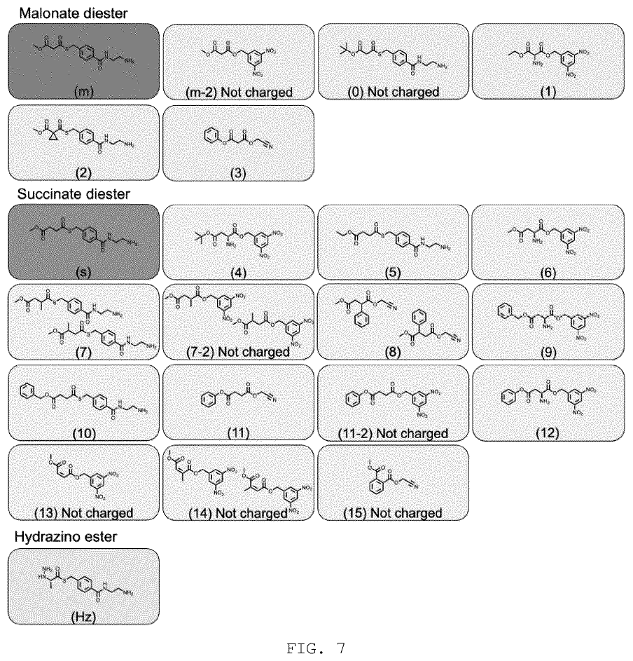

Diesters and hydrazines have been used as substrates to produce heterocyclic structures, where the diester serves as a scaffold for the cyclic structure and react with the hydrazine via a nucleophilic substitution reaction at the two reactive groups. This reaction, however, typically requires high temperatures and a catalyst (acid or base),^{42, 43} along with an excess of one of the reactants. Utilizing the ribosome as a catalyst for synthesizing this motif would provide a novel platform, enabling rapid synthesis in an aqueous solution under moderate temperatures (30-37° C.) as well as taking advantage of the ribosome's template-guided sequence-specific polymerization capability. To design a diester substrate compatible with Fx, the inventors first converted an acid moiety to an ester containing an FLG (FIG. 3A). The inventors next derivatized the other acid with different R₁ substituents that vary the steric hindrance and electron density around the electrophilic carbonyl carbon. R₁ substituents include tert-butyl, ethyl, methyl, benzyl, and phenyl groups. The choice of R₁ allows the rate and selectivity of the cyclization reaction to be finely controlled. The inventors also varied substituent R₂ (FIG. 3A) with aliphatic, branched-chain, and aromatic variants, demonstrating that ribosome-mediated synthesis can produce a diverse array of heterocyclic derivatives more efficiently than the conventional solution-based chemistry. Furthermore, the inventors varied the carbon chain length (malonate; 3-carbon chain) and succinate; 4-carbon chain), which produces different ring sizes once cyclization occurs (FIG. 3B). As a synthon for forming cyclic structures, the inventors employed 2-hydrazinylpropanoic acid; Hz, FIG. 3A), which also exhibits bifunctional nature due to its two nucleophilic nitrogen atoms (FIG. 7).

Fx-mediated acylation of ncMs and ribosome-mediated synthesis of cyclic motifs

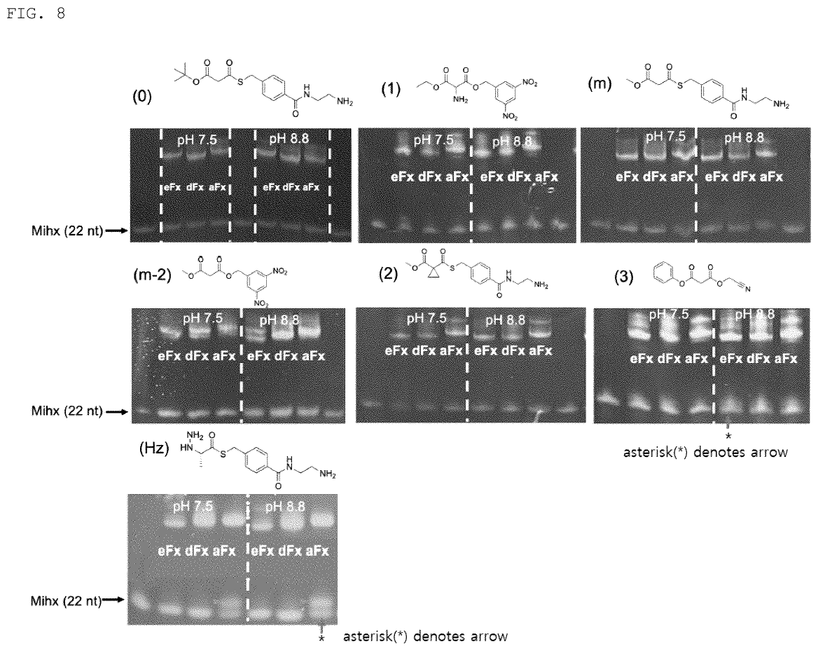

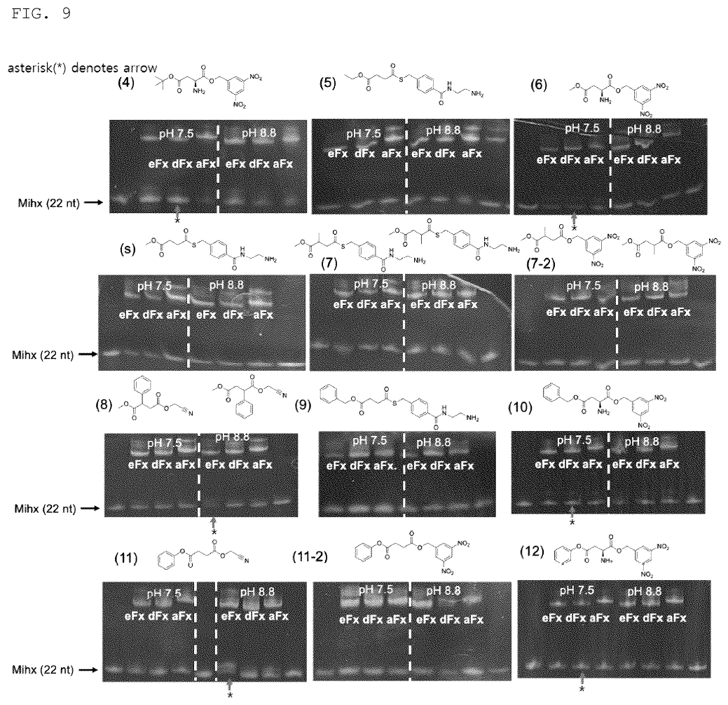

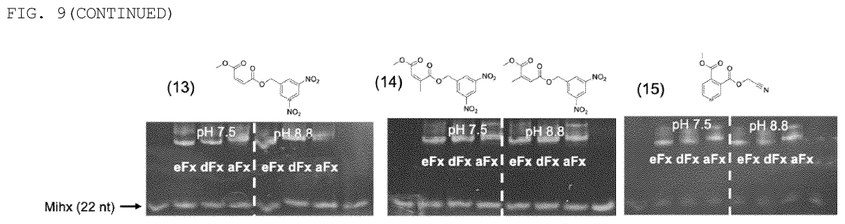

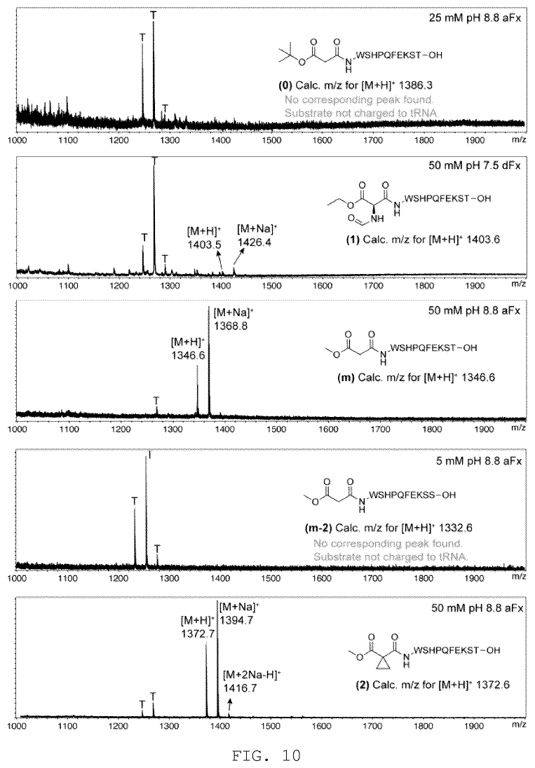

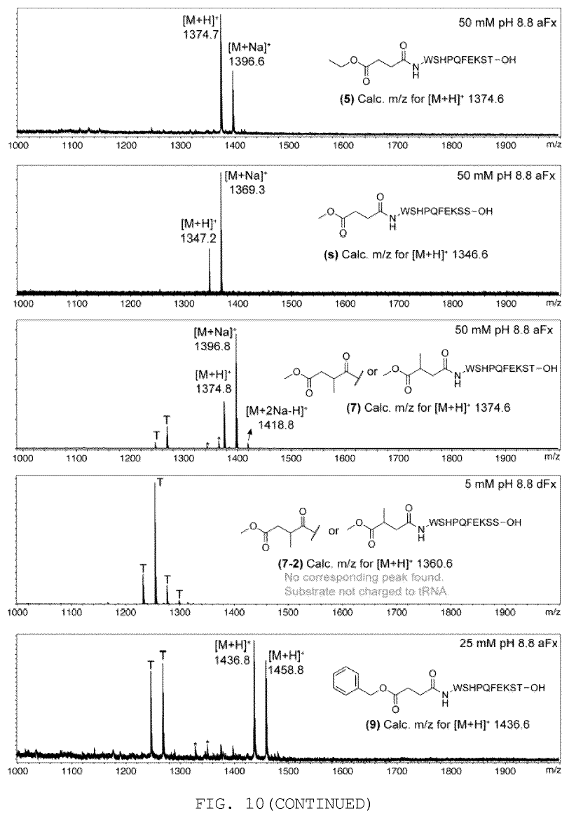

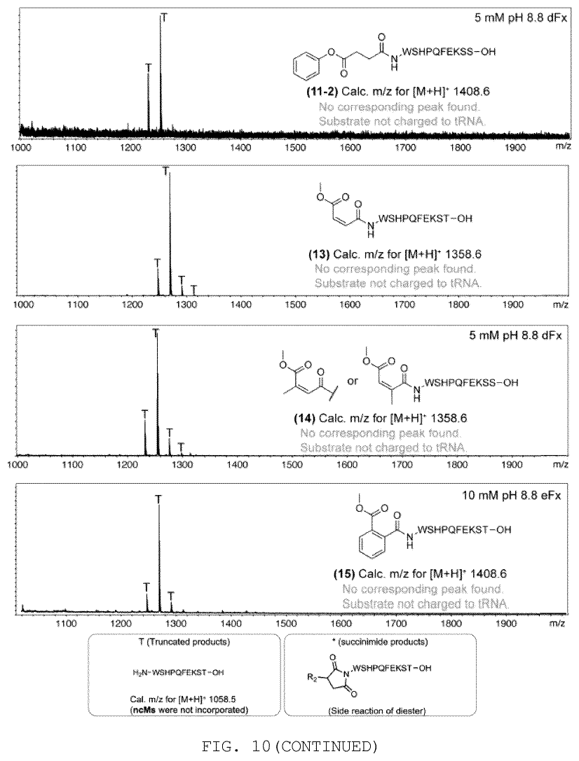

The inventors conducted Fx-mediated reaction in two stages to charge ncMs onto tRNAs. Initially, the inventors used a short microhelix tRNA (mihx), a 22-nucleotide tRNA analog, to determine the optimal conditions for acylation. This method offers rapid analysis because acylated mihx, with its increased molecular weight, can be distinguished from unacylated mihx due to their differing mobility on a polyacrylamide gel (FIGS. 8 and 9). For each ncM, six different experimental conditions were tested, varying pH levels (7.5 and 8.8) and Fx variants (eFx, dFx, and aFx) to identify the condition with the highest acylation yield.¹⁶ The inventors quantitatively assessed the acylation yield through densitometric analysis of gel bands. Once optimal conditions are identified, the inventors acylate a full length tRNA molecule with the respective ncM.

The inventors first charged substrate m (a malonic ester derivative) and Hz to microhelix (FIG. 4A). Substrate Hz achieved over 50% acylation, whereas substrate m did not show any significant band shift on the polyacrylamide gel. This lack of shift is presumably due to the faster electrophoretic mobility of substrate m in the acidic gel (pH 5.2), which is attributed to the absence of a positively charged moiety (e.g., —NH₃⁺) (FIGS. 8 and 9). Acylation of substrate m to tRNA by Fx is essential for achieving ribosome-mediated biosynthesis of new chemical bonds through two consecutive incorporations of both substrates. Thus, the inventors sought to measure successful acylation of substrate m to tRNA through more sensitive means.

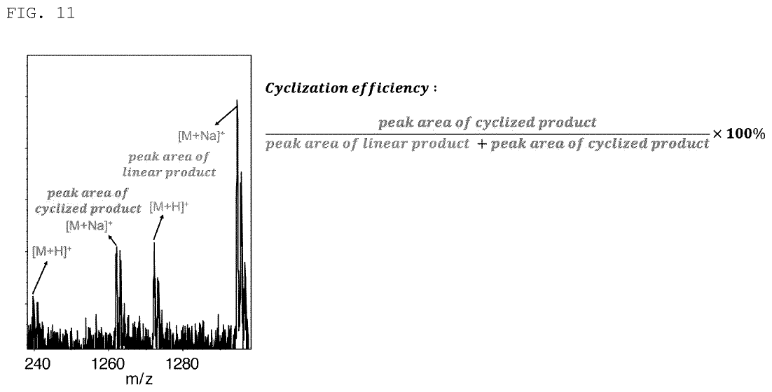

To achieve this, the inventors performed a Fx-mediated acylation reaction with substrate m to tRNA^{me}t(CAU). For ribosome-mediated biosynthesis, the inventors purified the acylated tRNA through ethanol precipitation to remove any unreacted substrate m. The resulting m: tRNA^{fmet} (CAU) conjugate was then re-suspended in an aqueous buffer and introduced into PURExpress™ (NEB), a reconstituted in vitro protein synthesis system. To facilitate the analysis of the products, the inventors designed a reporter gene producing a Strep tag (XWHSPQFEK) on a T7 promoter-controlled DNA plasmid (pJL1_X-StrepII), where X indicates the position to which Fx-charged m is incorporated. The inventors selected the Strep tag due to its high affinity for Strep-Tactin and the ability to elute target peptides under mild denaturing conditions (0.1% SDS). The reaction was carried out at 37° C. for 2 h in the presence of the other eight amino acids required for synthesis of the peptide containing a Strep tag. The reaction products were purified, eluted, and subsequently characterized via MALDI-TOF mass spectrometry. The mass spectrum analysis identified a peak corresponding to the theoretical mass ([M+H]⁺=1212.5; [M+Na]⁺=1234.5; [M+2Na—H]⁺=1256.5) of the product containing m at the N-terminus (FIG. 10), confirming substrate m is charged to tRNA by Fx.

For substrate Hz, the inventors carried out Fx-mediated reaction for tRNA^{Pro1E2}(GAU) using the same method. An engineered proline-tRNA was chosen for its efficient incorporation of ncMs into peptides in previous studies. The resulting Hz:tRNA^{Pro1E2}(GAU) conjugate was introduced to a PURExpress reaction containing m:tRNA^{fMet} (CAU) and a DNA plasmid (pJL1_XY-StrepII). This template encodes a Strep tag (XZWHSPQFEK), containing the two ncMs at positions X and Z (FIG. 4B). The Met(AUG) and Ile(AUC) codons were selected for X and Z, because Met and Ile are not required for Strep tag synthesis (and hence lack competition in a PURExpress system), and AUG is necessary as the initiation codon in protein synthesis. The resulting peptide was characterized using MALDI-TOF mass spectrometry, showing a peak corresponding to the theoretical mass ([M+H]⁺=1212.5; [M+Na]⁺=1234.4; [M⁺2Na—H]⁺=1256.3) of the product containing a pyrazolidinedione at the N-terminal region (upper panel in FIG. 4C). To the best of our knowledge, this marks the first demonstration of a ribosome-catalyzed synthesis of a 5-membered cyclic structure.

Exploring further, the inventors next tested ribosomal synthesis utilizing a wide spectrum of monomers containing diverse non-canonical backbones for incorporation into a growing polymer by the ribosome (FIG. 4A). The inventors focused on diesters containing an extended carbon chain capable of forming larger cyclic structures through the ring-closure mechanism with substrate Hz inside the ribosome (FIG. 4B). For this, the inventors chose succinic ester and designed a derivative (s) to have the same R₁ substituent. The inventors then found an optimal acylation condition (pH 8.8 and aFx) for tRNA charging by carrying out the Fx/mihx experiments and mass spectrometric analysis (FIGS. 8 to 10). With Fx-charged s:tRNA^{fMet} (CAU) with Hz:tRNA^{Pro1E2}(GAU), the inventors carried out in vitro translation using PURExpress™ and characterized the resulting product using mass spectrometry. The MALDI spectrum showed a peak corresponding to the theoretical mass of the peptide containing a 6-membered pyrazolidinedione at the N-terminus ([M⁺H]⁺=1226.5; [M⁺Na]⁺=1248.5, lower panel in FIG. 4C). Although a previous study reported the 6-membered structure with a pyridazinone (C₄H₆N₂O) motif produced by the ribosome, our invention has discovered a new cyclic structure with the pyrazolidinedione motif (C₄H₆N₂O₂).

Intriguingly, the mass spectrum (FIG. 4D) showed major peaks that did not correspond to the expected mass of product containing the 6-membered structure. The mass analysis suggests that the major peak (denoted as T) corresponded to the theoretical mass of a truncated peptide without any synthetic substrates at the N-terminus. However, the peak L matched the peptide containing substrate s, with a peptide bond connected to a single nitrogen atom from substrate Hz (FIG. 4B).

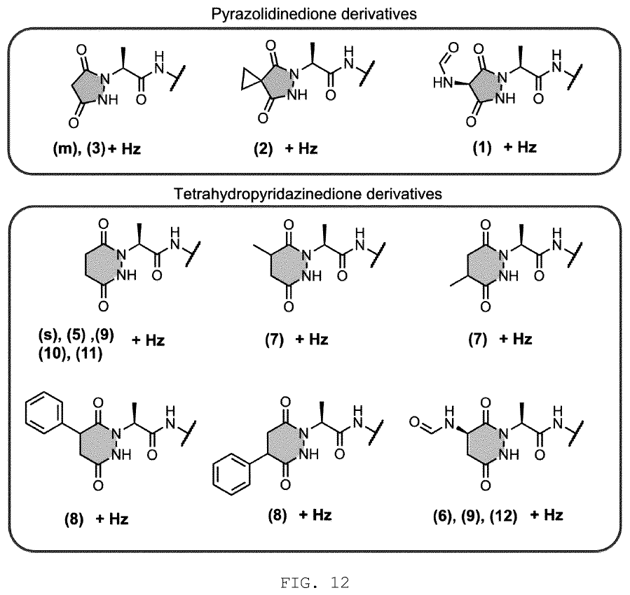

The inventors also considered the scenario in which the appropriate substrates are incorporated into the peptide, but the ring closing reaction is not efficiently carried out, resulting in a linear product (FIG. 4B). To determine the ratio of cyclic to linear products, the inventors calculated the percentage based on peak areas, assuming a calibration factor of 1.0 (FIG. 11). Based on this calculation, the inventors found that only 20% of the cyclic product was produced from substrate s (FIG. 4D), while the reaction with substrate m yielded no linear product (FIG. 4C), indicating nearly 100% cyclization efficiency. The inventors hypothesized that this is due to the fast kinetics of 5-membered ring formation, attributed to reduced ring strain and a stable transition state, as observed in solution chemistry.^{46, 47} To test this hypothesis further, the inventors looked to experiments that allowed us to tune the kinetic rate of ring formation.

Mechanistically, the formation of cyclic structure between a diester and a hydrazinoester, two sequential aminolysis reactions must occur within the ribosome. This involves release of both tRNA and alkoxide (R₁O⁻) from the two carbonyls of diester substrates. In the two reactions using substrates m and s, methoxide (CH₃O⁻), a conjugate base of methanol, with high basicity (pKₐ of ˜16) is expelled as a leaving group. The inventors hypothesized that high basicity makes methoxide less prone to dissociate and that altering the R₁ substituent to groups with different pKₐ may affect the kinetics of ring-closure reaction. To test this hypothesis, the inventors designed malonate and succinate derivatives with various R₁ groups (tert-butyl, ethyl, benzyl and phenyl) and various pKₐs (FIGS. 3A and 4A).

The cyclization kinetics are influenced by the R₁ substituents.

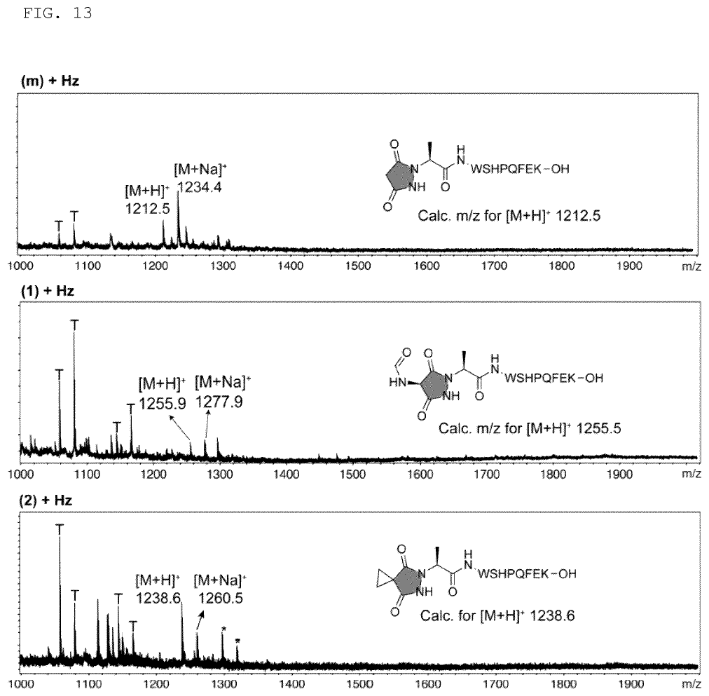

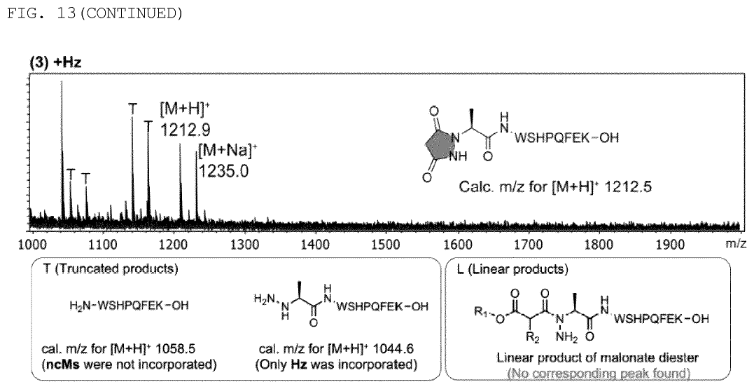

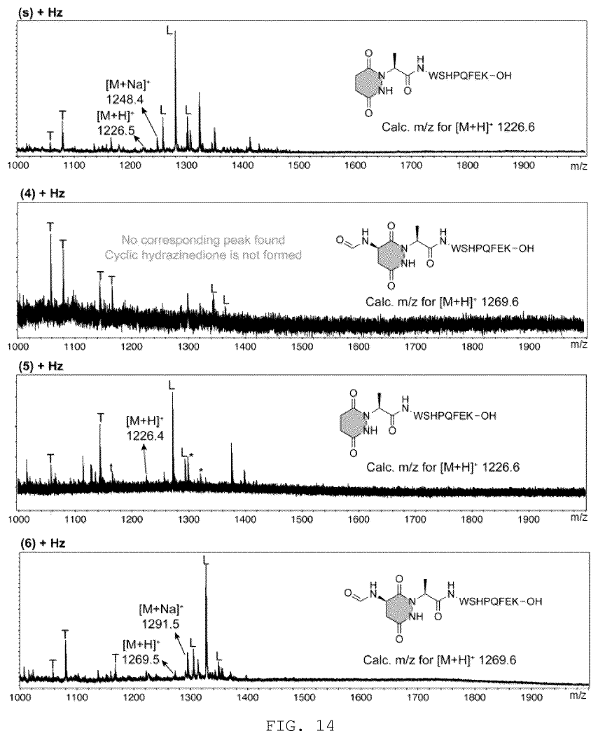

First, the inventors synthesized a range of malonic ester derivatives (substrates 1-3), varying the R₁ group (ethyl (1), methyl (2), and benzyl (3)) to evaluate its impact on forming 5-membered rings (FIG. 4A). The inventors also varied R₂ groups (R₂ ═H, amino, and cyclopropyl) (FIG. 12) to test if the ribosome's potential to catalyze the synthesis of 5-membered pyrazolidinedione derivatives is independent of an additional functional group attached to the carbon chain. After Fx-mediated tRNA acylation reaction and subsequent ribosomal synthesis with Hz:tRNA in the PURExpress system, the inventors characterized the resulting products by MALDI mass spectrometry. In the MALDI spectrum (FIG. 5A (upper panel) and FIG. 13), a peak corresponding to the peptide containing the desired 5-membered ring structure was observed. Notably, peaks indicative of the linear form were absent across the different R₂ substituents (i.e. substrates 1, 2, and 3). This suggests that the basicity of the R₁ substituent, which ranges across seven orders of magnitude in pKₐ (from phenolate to ethoxylate), and the bulkiness/basicity of the R₂ substituent have minimal impact on the efficiency of 5-membered ring formation within the ribosome. This is presumably because the rapid kinetics of ring formation minimizes the potential impact of leaving groups. Moreover, the presence of additional groups (R₂) adjacent to the reaction site does not impede the kinetics of ring formation (FIG. 5B and FIG. 13).

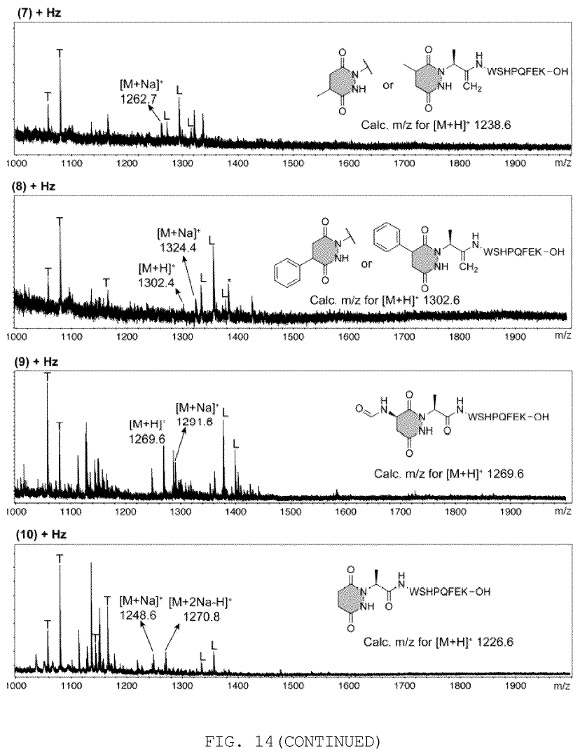

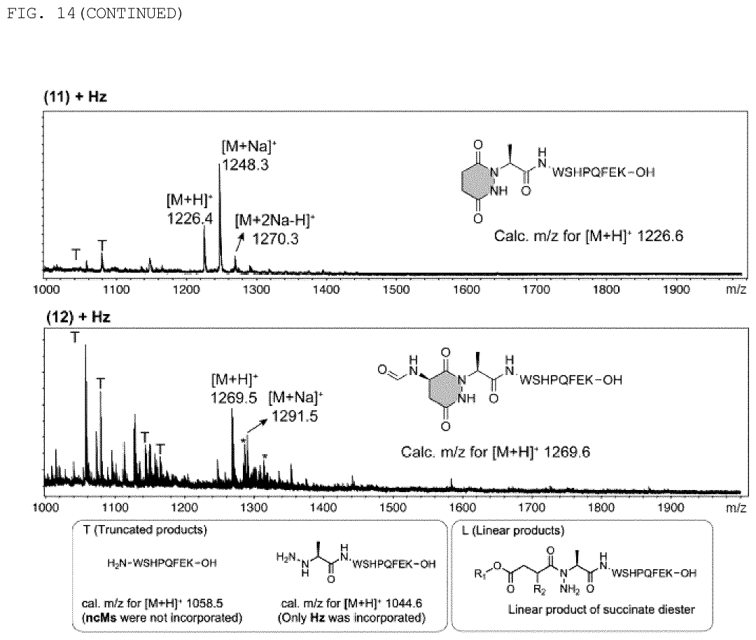

The inventors next examined the impact of R₁ substituents on the formation of 6-membered rings. To explore this, the inventors used nine succinic ester derivatives (substrates 4-12) with different R₁ groups (FIG. 4A). Notably, substrate 4 containing a tert-butyl substituent yielded no cyclized product (cyclization efficiency: 0%, FIG. 4A, FIG. 14). The inventors hypothesized that this was due to the following two key factors: i) the tert-butyl group creates significant steric hindrance, which impedes nucleophilic attack on the carbonyl carbon, a rate-determining step in aminolysis, and ii) the tert-butoxylate has a higher basicity (pKₐ ˜17) and thus is less favorably displaced from the carbonyl carbon compared to substrate s containing a methoxy substituent (FIGS. 4A and 5B). In this context, a less bulky or less basic substituent may improve the cyclization efficiency. To test this hypothesis, the inventors designed substrates 5-8 with ethyl or methyl groups (pKₐ ˜16). These products showed an increased cyclization efficiency of ˜30±5% in the formation of 6-membered ring. Substrates 9 and 10, bearing a benzyloxylate with pKₐ of ˜15 for the R₁ substituent demonstrated cyclization efficiencies of 40% and 45%, respectively (FIG. 14). Interestingly, substrates 11 and 12, which contain a phenolate (pKₐ of ˜10) as the R₁ substituent, exhibited a singular peak corresponding to a peptide with a 6-membered ring in the MALDI spectrum, indicating a cyclization efficiency of 100% (FIG. 5C). This demonstrates that substituent choice can be an effective strategy to tune ring formation efficacy, with weaker bases better facilitating the ring formation reaction within the ribosome's active site. This result supports our hypothesis that the weaker phenolate stabilizes the intermediate through more efficient deprotonation, thereby accelerating the rate-limiting step of the cyclization process (FIG. 15). In summary, the inventors synthesized 14 new peptides containing pyrazolidinedione and tetrahydropyridazinedione backbones using the rapid ribosomal synthesis. This backbone chemistries have not been achieved before in ribosomal synthesis, and take place under milder reaction conditions (aqueous buffer, 37° C.) compared to other organic reactions used to produce same motif.

Cyclization does not occur in solution spontaneously.

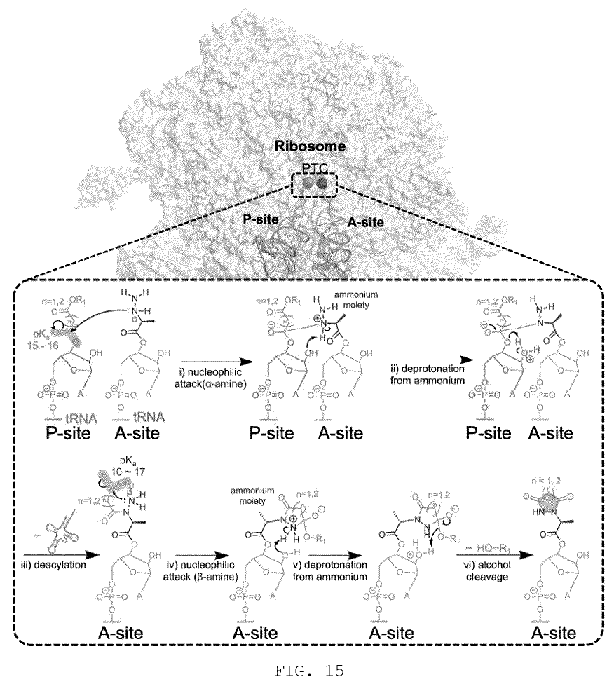

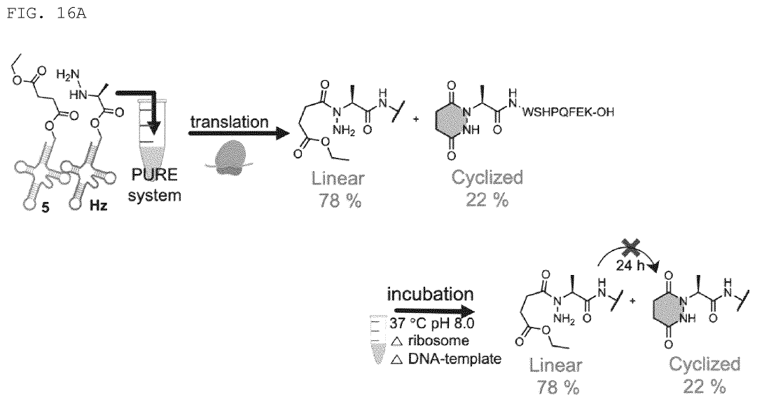

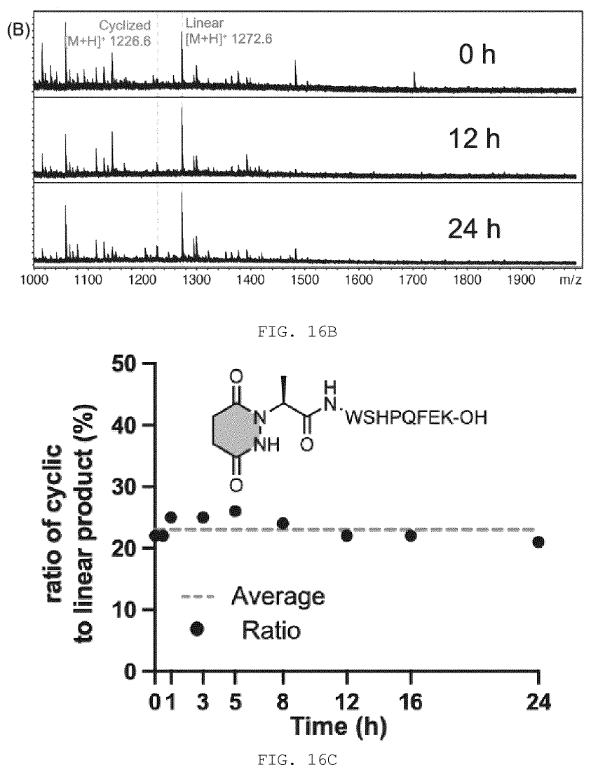

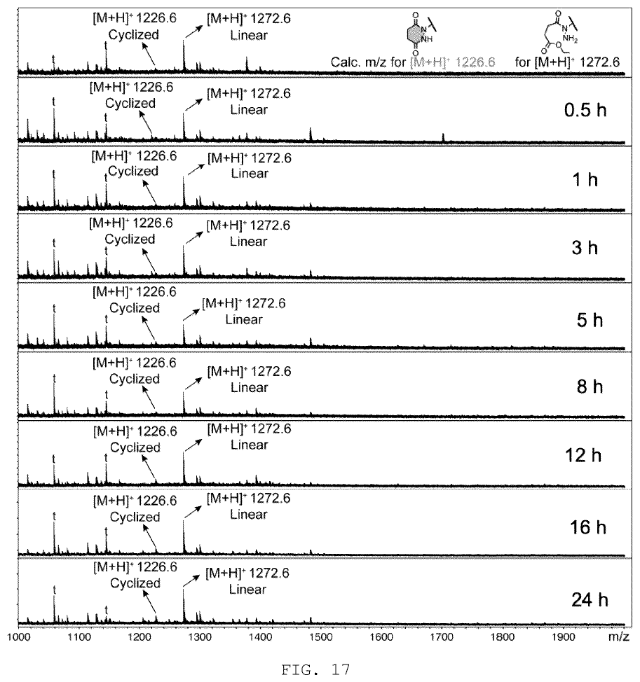

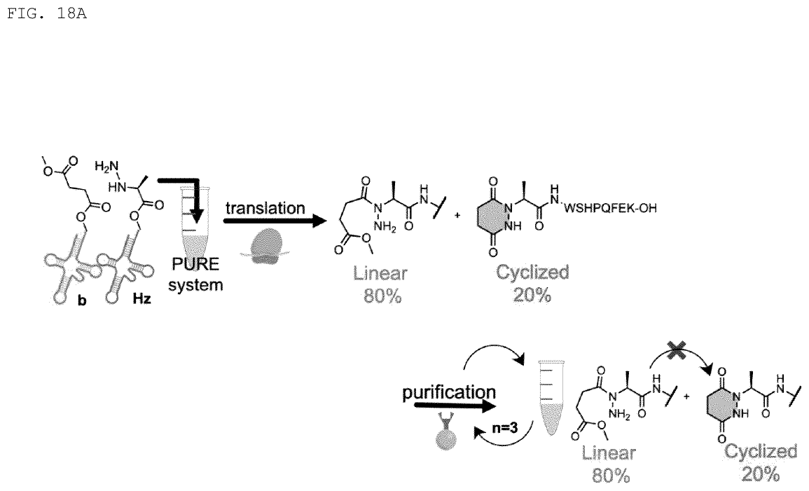

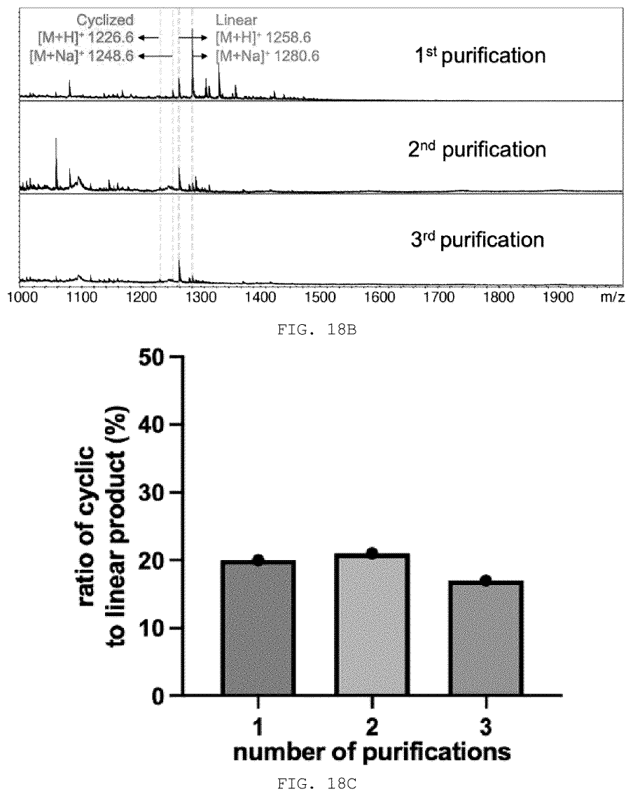

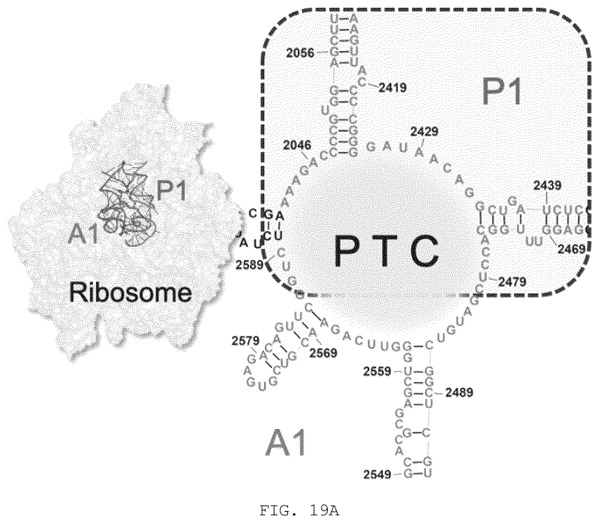

Our data demonstrate the ribosome's ability to form two novel cyclic hydrazinedione derivatives (FIGS. 3 to 5). The inventors considered the possibility that these cyclic structures could be formed through an intramolecular self-cyclization reaction in solution, rather than through ribosome-mediated catalysis. To investigate this, the inventors focused on the 6-membered ring produced with substrates 5 and Hz, which showed 22% cyclization efficiency (FIG. 4A). If cyclization were to occur spontaneously in solution, the inventors would expect an increasing proportion of cyclized products over time due to the gradual cyclization of linear products. To verify this, the inventors prepared a reaction under the same conditions used for cyclic structure synthesis and introduced the linear product into it. The ribosome and target DNA template were omitted to eliminate any potential for producing new cyclic products. The inventors monitored the product composition over 24 h through MALDI-TOF mass spectrometry. As shown in FIG. 16A and FIG. 17, the ratio of cyclized to linear product remained constant, suggesting that no cyclic products formed by a self-cyclization manner under these conditions (FIG. 16B). The inventors also considered whether additional materials (Strep-Tactin-coated magnetic beads) or chemicals (100 mM Tris-HCl, 150 mM NaCl, 1 mM EDTA, pH 8) used in the experiment might cause a self-cyclization reaction. The inventors used the product obtained from the reaction of s and Hz and carried out multiple magnetic capture-release experiments to simulate the purification procedure using the same buffer (FIG. 18). Not surprisingly, the inventors observed a constant ratio across cycles, further supporting that the observed cyclic structures are not the result of intramolecular self-cyclization during the experiments.

Ribosomal catalysis is required for cyclization.

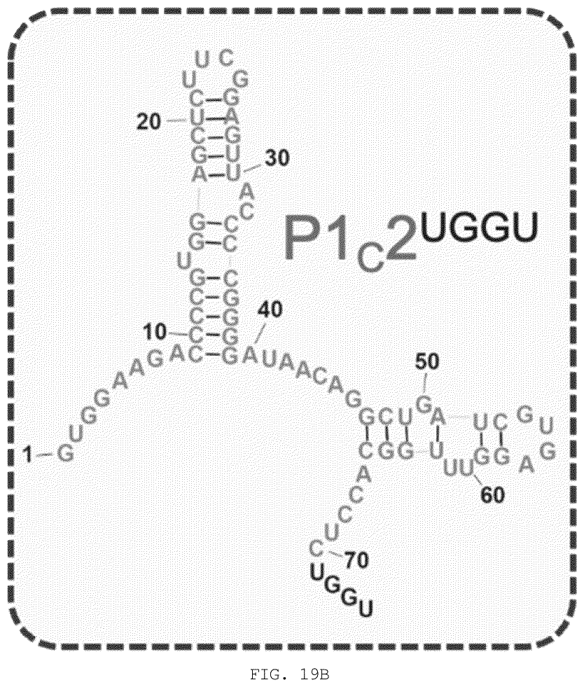

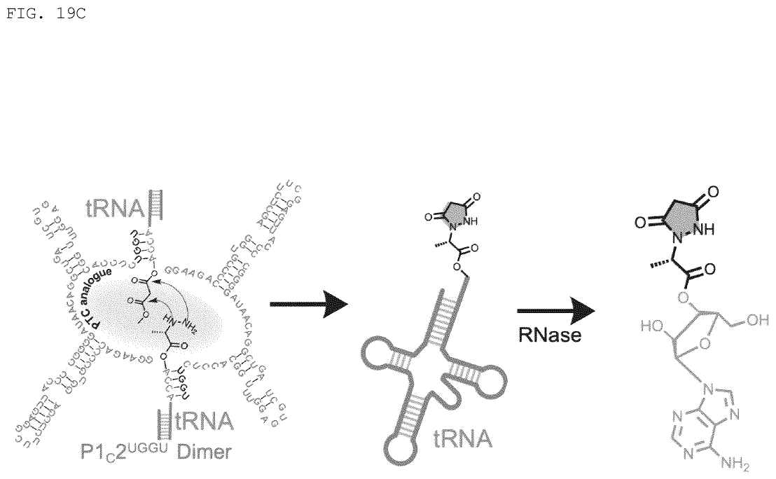

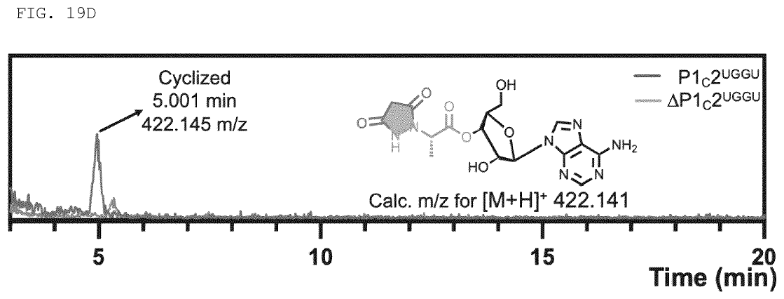

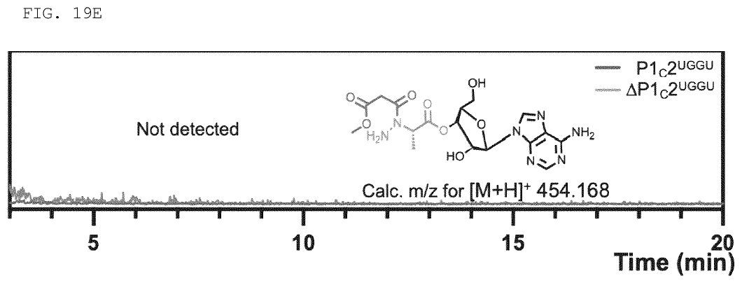

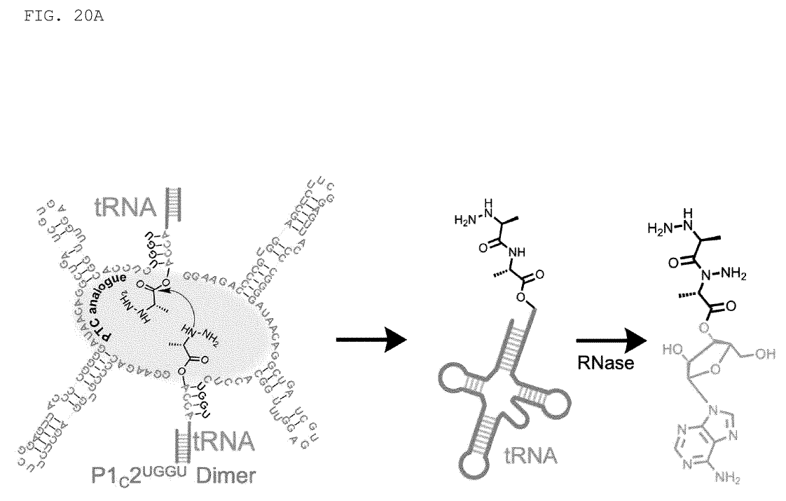

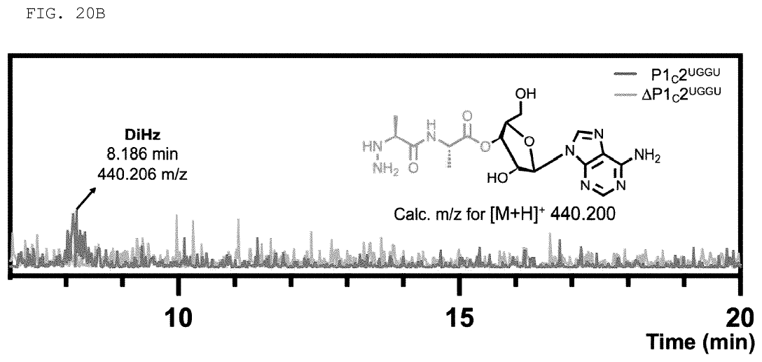

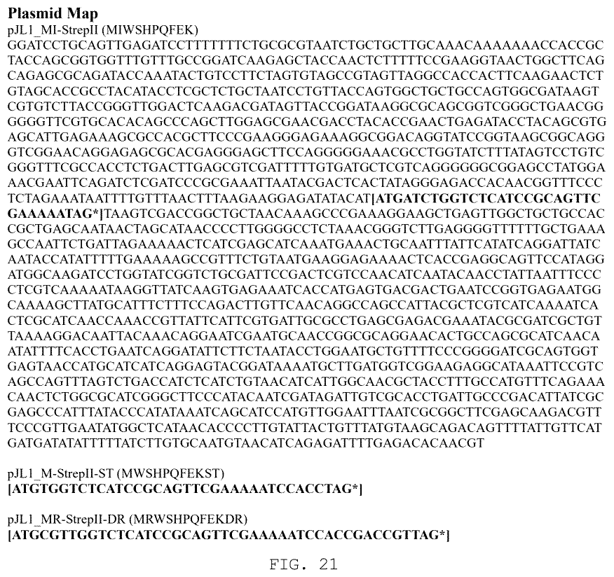

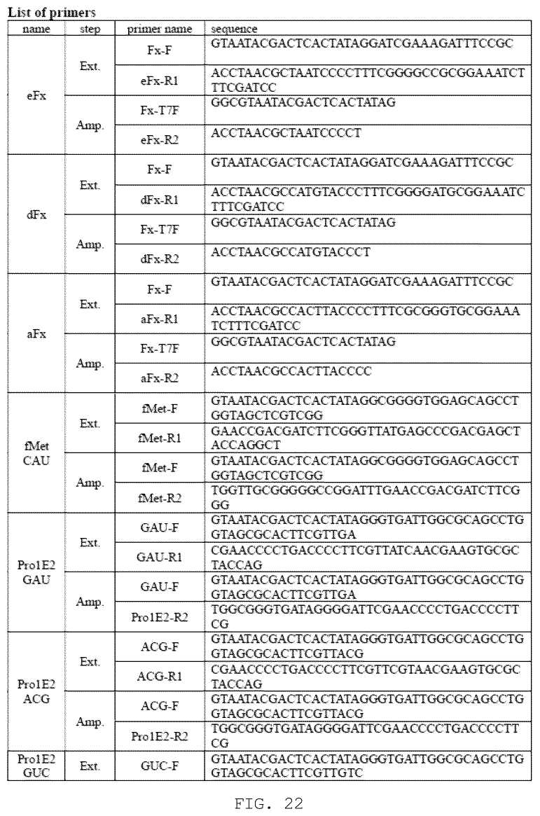

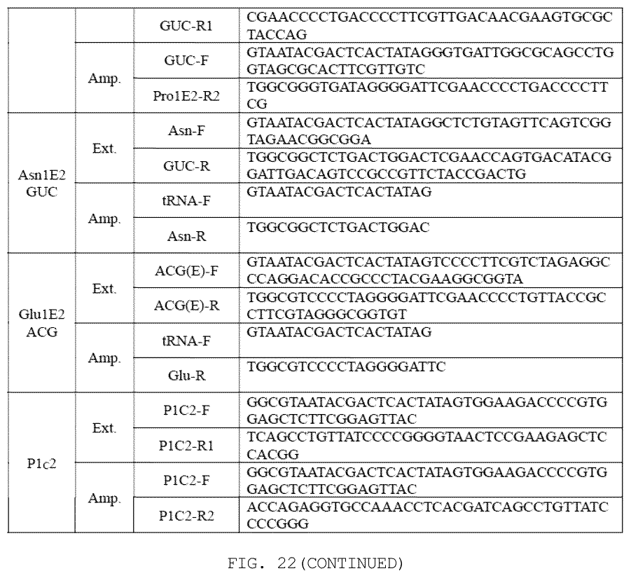

To directly demonstrate that the cyclic hydrazinedione motif results from the ribosome's catalytic activity, the inventors conducted an additional experiment using a functional RNA molecule that mimics the peptidyl transferase center (PTC) components of the E. coli 23S ribosomal RNA (FIG. 19A). Specifically, the inventors utilized the P1c2UGGU domain (74-mer), which incorporates elements from the P- and A-sites of the ribosome's domain V (FIG. 19B). This domain forms a dimer, creating a symmetrical three-dimensional structure similar to the ribosome's active site and capable of catalyzing peptide bond formation (FIG. 19C). The 3′-UGGU sequence was specifically designed to pair with tRNA molecules bearing a complementary 3′-ACCA sequence, thereby positioning the substrates close to the catalytic sites. The inventors synthesized P1c2^{UGGU} and tRNA^{ACCA} using in vitro transcription with T7 RNA polymerase. The inventors then separately charged substrates m and Hz to tRNA^{ACCA} via Fx, purified them, and introduced them to a solution containing P1c2^{UGGU}. After the reaction (24 h at 0° C.), the inventors digested the tRNA molecules with RNase (5 min, at room temperature) to isolate and monitor the product attached to the 3′-adenosine residue (FIG. 19C). The crude reaction mixture was analyzed using liquid chromatography-time-of-flight (LC-TOF) mass spectrometry. The extracted ion chromatogram yielded a single peak at 5.001 min corresponding to the mass of the 5-membered pyrazolidine-3,5-dione (calculated [M⁺H]⁺: 422.141, observed [M⁺H]⁺: 422.145; blue, FIG. 19D). No peak for the 5-membered hydrazinedione was detected in the absence of P1c2^{UGGU}, indicating that the formation of cyclic structure depends on catalysis by the P1c2 domain (gray, FIG. 19D). Also, no linearized product (calculated [M⁺H]⁺: 454.168) was observed either in the presence or absence of P1c2, which is consistent with the experiment carried out with the full ribosome (FIG. 19E). Notably, because P1c2 is a part of the large subunit (50S) of the ribosome, it lacks the function of positioning substrates sequentially through mRNA codons. As a result, the dimeric form of Hz-Hz was observed (FIG. 20).

### Mechanistic proposal for ribosomal synthesis of non-standard ring structure: the α-nitrogen links, and subsequently, the β-nitrogen cyclizes

The ribosome naturally prefers L-α-amino acids for peptide bond formation. Several pioneering studies have shown that the ribosome can incorporate β-amino acids, albeit with considerably reduced efficiency compared to α-amino acids. This reduced efficiency is presumably due to the extended carbon chain of β-amino acids, which hinders the optimal alignment of the β-nitrogen within the ribosome's peptidyltransferase center for an effective nucleophilic attack on the P-site tRNA. Substrate Hz, which has an NH₂ group linked to another NH₂ group at both the a and B positions, presents unique properties. In reactions involving hydrazine with carboxylic acid derivatives, the β-nitrogen is the primary atom used for bond-forming reactions due to steric hindrance around the α-nitrogen (FIG. 6A). This raises an important question: can the β-nitrogen outcompete the ribosome's inherent preference for using the α-nitrogen for peptide bond formation within the catalytic site?

The cyclic structures explored in this work have two possible regioisomers. In one scenario, the α-nitrogen atom of the incoming hydrazine ester attacks the ester of the P-site tRNA, while the β-nitrogen targets the ester carbonyl substituted with R₁ (FIG. 6A). Alternatively, the β-nitrogen atom might attack the tRNA linkage, reversing the roles of the nitrogen atoms (FIG. 6B). A critical distinction between these two scenarios is that the first scenario produces a free primary amine in the linear product, while the second scenario does not. This free amine remains reactive, allowing subsequent reactions such as a Schiff base reaction. To identify the regioselectivity involved, the inventors introduced 4-methylbenzaldehyde (4-mba) to the product resulting from the reaction of s and Hz, which consists ˜80% of the linear product (FIG. 6C). Mass spectrometry revealed a single peak, corresponding to the mass of a product containing a hydrazone moiety at the β-nitrogen position (FIG. 6C), suggesting that the α-nitrogen atom attacks the P-site tRNA as shown in Scenario 1 (FIG. 6A). Taken together, these findings confirm that, in ribosome-mediated cyclization, the α-nitrogen atom is used first for linking the nascent peptide chain, and subsequently the β-nitrogen atom undergoes ring closure with the ester carbonyl group substituted with R₁. Despite the lower steric hindrance of the β-nitrogen, the natural ribosome selectively uses the α-nitrogen for peptide bond formation (see FIG. 15 for a full proposed mechanism).

### Synthesis of non-ribosomal peptide-like products

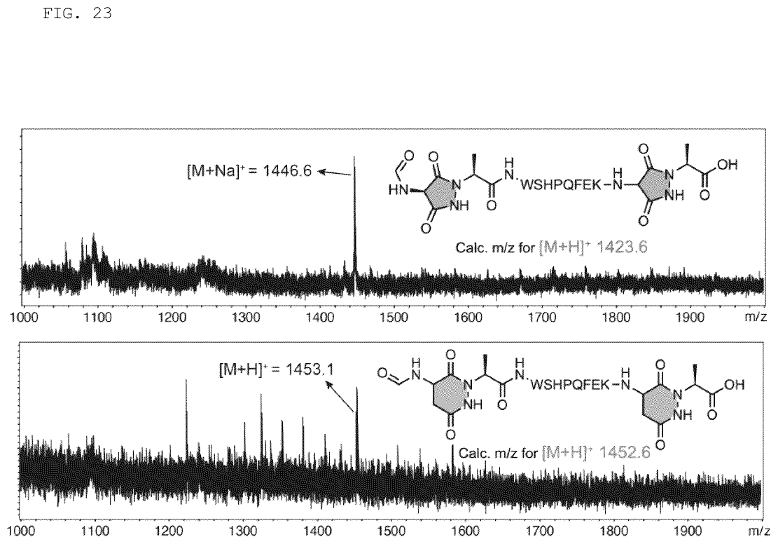

Direct production of non-ribosomal peptide-like materials may significantly enhance the diversity of peptide drug candidates in a library, particularly when prepared for screening via drug discovery platforms. For example, the RaPID (Random non-standard Peptides Integrated Discovery) system leverages the advantages of diverse cyclic structures, which are crucial for drug efficacy, with template-guided synthesis, inherent to ribosome-based synthesis. These structures contribute to improved thermodynamic stability, resistance to proteolytic degradation, and protein-protein interactions important in therapeutics. To test the ribosome's ability to produce non-ribosomal peptide-like materials with multiple alternative backbones in a peptide, the inventors designed an additional plasmid (pJL1-XZ-StrepII-X′Z) that allows site-specific, alternating incorporations of diester substrates and Hz. In a separate tube containing PURExpress™ (NEB) reaction materials, the inventors supplemented Fx-charged tRNA^{fMet} (CAU): 1 and tRNA^{Pro1E2} (GUC): 12, with tRNA^{Pro1E2} (ACG):Hz, respectively, for multiple ring-closing reactions. The mass spectra (FIG. 23) yielded peaks of the products containing multiple 5—and 6-membered cyclic backbones according to the mRNA sequence, suggesting the potential of synthesizing biopolymers that resemble non-ribosomal peptides used in drug discovery platforms.

### DISCUSSION

In this work, the inventors demonstrate ribosome-mediated biosynthesis of non-standard cyclic motifs using rationally designed diester and hydrazine substrates (non-canonical monomers, ncMs). These ncMs were charged to tRNA by an acylating-ribozyme then consecutively incorporated into a peptide, with the ribosome catalyzing multiple aminolysis reactions in its catalytic site. Through a series of chemical reactions and mass spectrometric analysis, the inventors revealed that the α-nitrogen atom of the hydrazine monomer extends the monomer onto the nascent peptide chain, while the β-nitrogen undergoes a ring-closing reaction in the ribosome. By varying the length of the monomer scaffolds, the inventors achieved the formation of diverse five- and six-membered cyclic backbones on a peptide. Cyclization efficiencies between 0 to 100% were observed through rational design of ester substituents.

The inventors believe this breakthrough bridges the gap between the chemical advantages of cyclic motifs in drug-like peptides with the ease of synthesis using the translation machinery, catalyzed by the ribosome. To our knowledge, this is the first demonstration of the ribosome's ability to modulate ring-closing reactions, leveraging the inherent biochemical properties of ncMs. Looking ahead, this platform could greatly accelerate the synthesis of heterocyclic motifs. For instance, a previous study showed that incorporating a 6-membered tetrahydropyridazinedione into a peptide required a seven-step process under highly acidic conditions.⁶⁰ In contrast, our approach using the ribosome in vitro produces the same motif in an aqueous buffer within 2 h at 37° C. This capability could transform the synthesis of the five-membered pyrazolidine motif, already used in small-molecule drugs.⁶¹⁻⁶⁵ It could also enhance the production of novel peptides library used for drug discovery platforms like RaPID. By integrating diverse cyclic structures into the range of peptidomimetics, this approach holds potential to accelerate the development of new therapeutics.

Interestingly, the reactions occurring within the ribosome appear to follow conventional chemical principles found in solution. For example, five-membered ring formation is faster than six-membered ring formation, and cyclization yield depends on the stability of the leaving group. However, kinetic studies (e.g., entropy, enthalpy, and reaction rates for the ribosome) remain largely unexplored. The inventors expect further kinetic investigations to improve our understanding of the ribosome's catalytic core, enabling the in vitro synthesis of an even greater array of cyclic structures. Non-canonical monomer polymerization efficiency could potentially be improved by incorporating an updated set of translational components, such as EF-P, EF-Tu, tRNAs, and ribosomes specially tailored to accommodate ncMs. Future developments in this field may expand the spectrum of possible chemical reactions, potentially transforming our understanding of polymerization chemistry and producing the next generation of medicinal molecules.

The scope of the present invention is indicated by the claims described later rather than the detailed description above. All changes or modified forms derived from the meaning and scope of the patent claims and their equivalent concepts should be construed as being included in the scope of the present invention.

What is claimed is:

1. An acylated tRNA represented by Structural Formula 1 or Structural Formula 2 below: 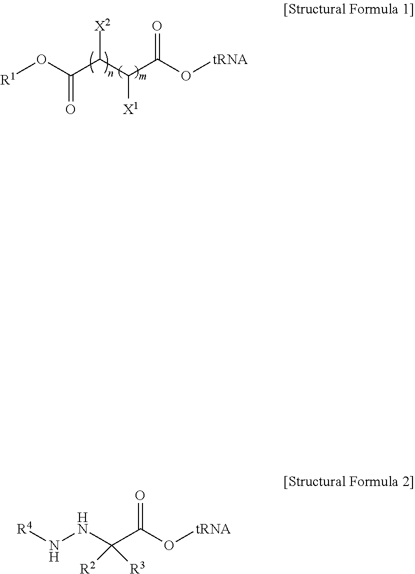
wherein R¹ is a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group, or a substituted or unsubstituted (C6-C14 aryl)(C1-C8 alkyl) group,
X¹ and X² are each independently a hydrogen atom, a substituted or unsubstituted C1-C10 alkyl group, a substituted or unsubstituted C3-C7 cyclic alkyl group, 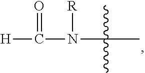
a substituted or unsubstituted C6-C14 aryl group, a substituted or unsubstituted (C1-C8 alkyl)(C6-C14 aryl) group, a substituted or unsubstituted (C6-C14 aryl)(C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and Rand R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group, and
a group suitable for substitution on the R¹, X¹ and X², and R⁴ is identical or different and represents a C1-C5 alkyl group or C6-C12 aryl group, and a group suitable for substitution on the R² and R³ is identical or different and represents a C1-C5 alkyl group, C6-C12 aryl group, a hydroxyl group or an amino acid group.

2. The acylated tRNA of claim 1, wherein the acylated tRNA is represented by the Structural Formula 1 and comprises at least one selected from the group consisting of compounds below:

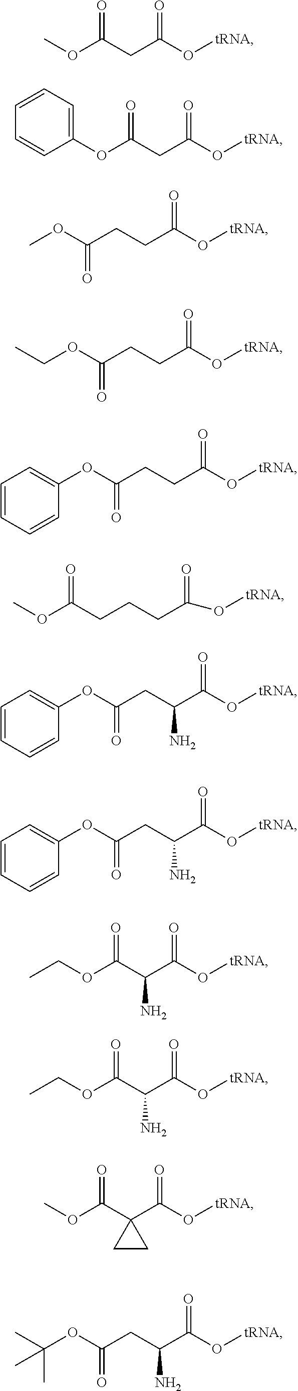

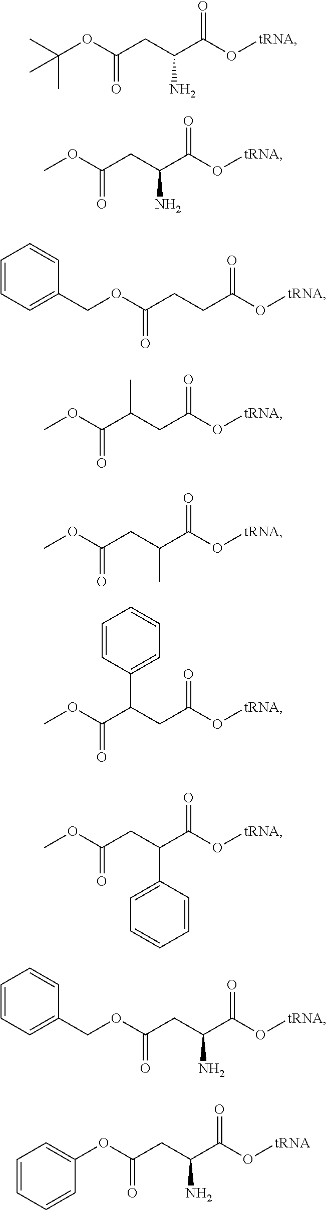

3. The acylated tRNA of claim 1, wherein the acylated tRNA is represented by the Structural Formula 2 and comprises at least one selected from the group consisting of compounds below:

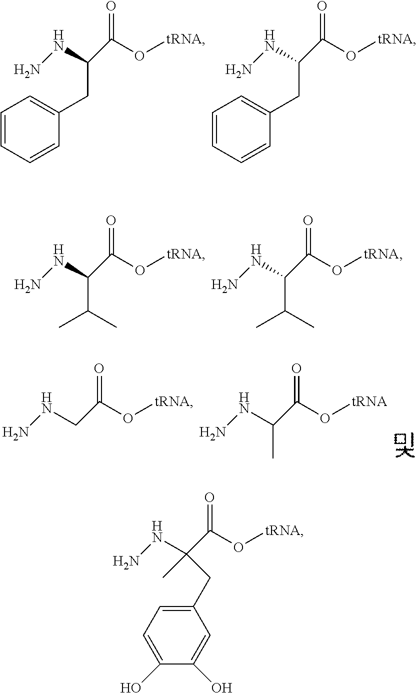

4. The acylated tRNA of claim 1, wherein the acylated tRNA is represented by the Structural Formula 2 and comprises at least one selected from the group consisting of compounds below:

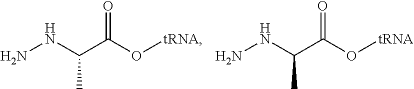

5. The acylated tRNA of claim 1, wherein the acylated tRNA is represented by the Structural Formula 2 and comprises at least one selected from the group consisting of compounds below:

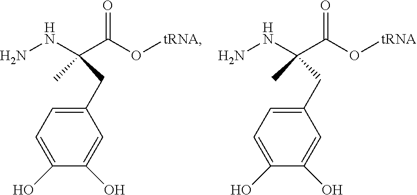

6. A cyclic compound represented by structural formula 3 below: 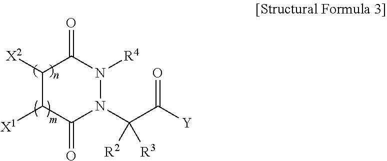
wherein X¹ and X² are each independently a hydrogen atom, a substituted or unsubstituted C1-C10 alkyl group, a substituted or unsubstituted C3-C7 cyclic alkyl group, 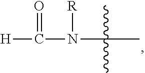
a substituted or unsubstituted C6-C14 aryl group, a substituted or unsubstituted (C1-C8 alkyl)(C6-C14 aryl) group, a substituted or unsubstituted (C6-C14 aryl)(C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and Rand R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group,
Y is a hydrogen atom, —O(tRNA), —O(RS), —N(R⁶)(R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group, and
a group suitable for substitution on the X¹ and X², R⁴ and R⁵ to R⁷is identical or different and represents a C1-C5 alkyl group or C6-C12 aryl group, and a group suitable for substitution on the R² and R³ is identical or different and represents a C1-C5 alkyl group, C6-C12 aryl group, a hydroxyl group or an amino acid group.

7. The cyclic compound of claim 6, wherein the cyclic compound comprises at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group, 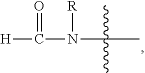
a substituted or unsubstituted C6-C14 aryl group, a substituted or unsubstituted (C1-C8 alkyl)(C6-C14 aryl) group, a substituted or unsubstituted (C6-C14 aryl)(C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and Rand R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶)(R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group, and
a group suitable for substitution on the X¹ and X², and R⁵ to R⁷is identical or different and represents a C1-C5 alkyl group or C6-C12 aryl group:

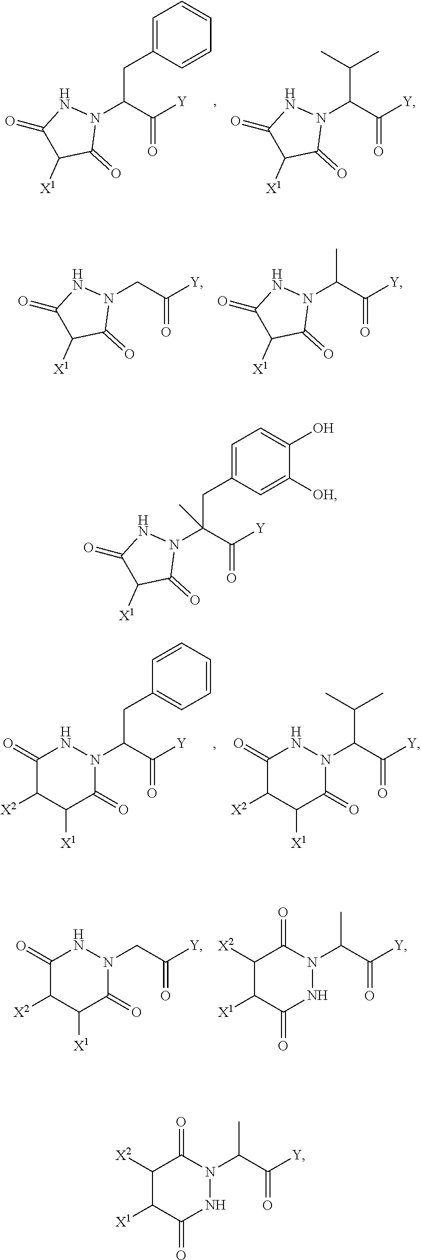

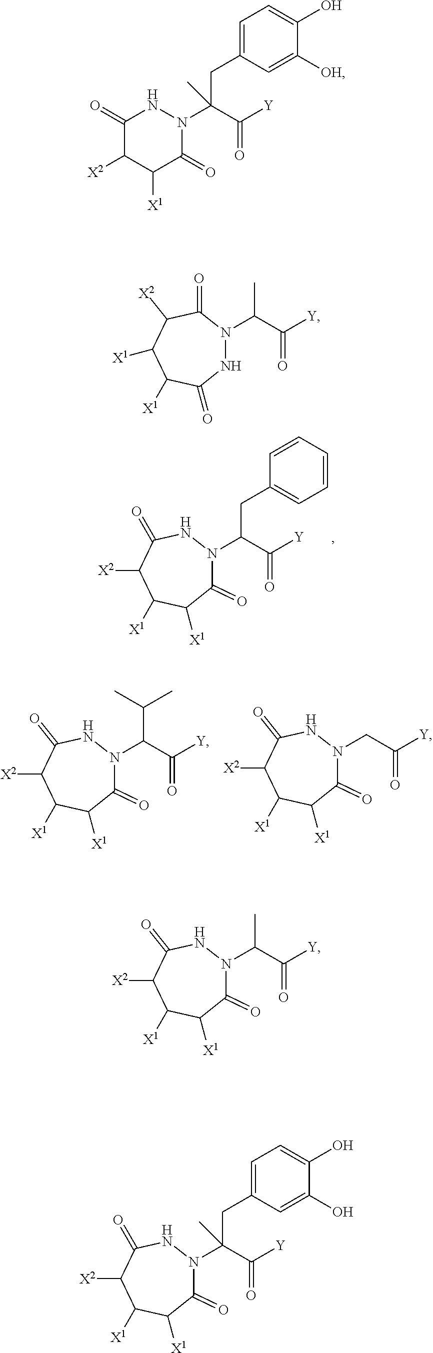

8. The cyclic compound of claim 6, wherein the cyclic compound comprises at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group, 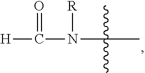
a substituted or unsubstituted C6-C14 aryl group, a substituted or unsubstituted (C1-C8 alkyl)(C6-C14 aryl) group, a substituted or unsubstituted (C6-C14 aryl)(C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and Rand R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶)(R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group, and
a group suitable for substitution on the X¹ and X², and R⁵ to R⁷is identical or different and represents a C1-C5 alkyl group or C6-C12 aryl group:

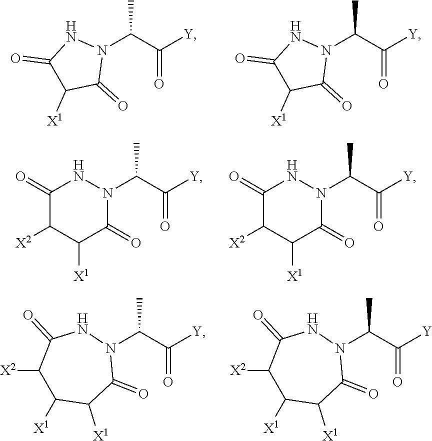

9. The cyclic compound of claim 6, wherein the cyclic compound comprises at least one selected from the group consisting of compounds below,
wherein X¹ and X² are each independently a hydrogen atom, a substituted or unsubstituted C1-C10 alkyl group, a substituted or unsubstituted C3-C7 cyclic alkyl group, 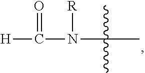
a substituted or unsubstituted C6-C14 aryl group, a substituted or unsubstituted (C1-C8 alkyl)(C6-C14 aryl) group, a substituted or unsubstituted (C6-C14 aryl)(C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and R′ and R″ are each independently a hydrogen atom, a substituted or unsubstituted C1-C8 alkyl group or a substituted or unsubstituted C6-C14 aryl group,
Y is a hydrogen atom, —O(tRNA), —O(R⁵), —N(R⁶)(R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group, and
a group suitable for substitution on the X¹ and X², and R⁵ to R⁷is identical or different and represents a C1-C5 alkyl group or C6-C12 aryl group:

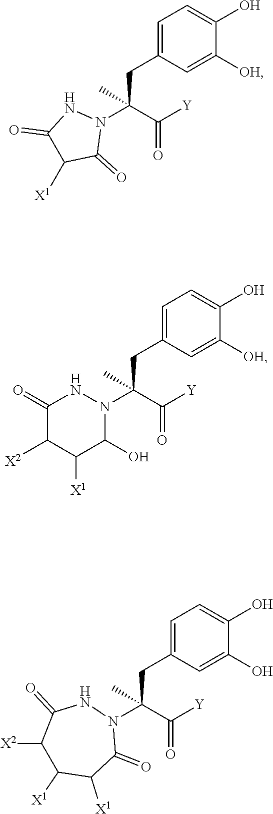

10. A method for cyclizing an unnatural substrate comprising a step of conjugating an acylated tRNA represented by Structural Formula 1 and an acylated tRNA represented by Structural Formula 2 in a translation reaction to produce a cyclic compound represented by structural formula 3. 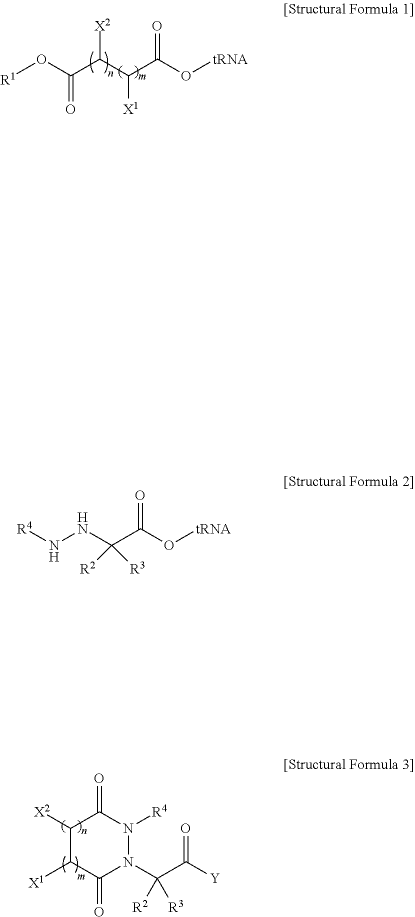
wherein R¹ is a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group, or a substituted or unsubstituted (C6-C14 aryl)(C1-C8 alkyl) group,
X¹ and X²are each independently a hydrogen atom, a substituted or unsubstituted C1-C10 alkyl group, a substituted or unsubstituted C3-C7 cyclic alkyl group, 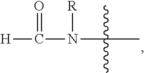
a substituted or unsubstituted C6-C14 aryl group, a substituted or unsubstituted (C1-C8 alkyl)(C6-C14 aryl) group, a substituted or unsubstituted (C6-C14 aryl)(C1-C8 alkyl) group, an amino group, or an amine group represented as —NR′R″, or at least one of X¹ and X² are combined with a carbon in a main chain to form a substituted or unsubstituted C3-C6 ring, wherein R is a hydrogen atom or a C1-C5 alkyl group, and Rand R″ are each independently a hydrogen atom, a substituted or unsubstituted C1-C8 alkyl group or a substituted or unsubstituted C6-C14 aryl group,
m is 0, 1 or 2,
n is 0, 1 or 2,
m and n are not 0 at the same time,
m and n are not 2 at the same time
R² and R³ are each independently a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group,
R⁴ is a hydrogen atom, a substituted or unsubstituted C1-C10 alkyl group, a substituted or unsubstituted C3-C7 cyclic alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group,
Y is a hydrogen atom, —O(tRNA), —O(R⁵), —N(R⁶)(R⁷), or a polymer chain covalently bonded to a neighboring carbonyl group,
R⁵ to R⁷ are each independently a substituted or unsubstituted C1-C10 alkyl group, a substituted or unsubstituted C6-C14 aryl group, a substituted or unsubstituted (C1-C8 alkyl)(C6-C14 aryl) group or a substituted or unsubstituted (C6-C14 aryl)(C1-C8 alkyl) group, and
a group suitable for substitution on the R¹, X¹ and X², R⁴ and R⁵ to R⁷ is identical or different and represents a C1-C5 alkyl group or C6-C12 aryl group, and a group suitable for substitution on the R² and R³ is identical or different and represents a C1-C5 alkyl group, C6-C12 aryl group, a hydroxy group or an amino acid group.
